SEQUENCE LISTING (108 sequences)

SEQ ID NO: 1 - DNA, 469 bp, Homo sapiens
ctgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcag

SEQ ID NO: 2 - DNA, 1733 bp, Homo sapiens
ctcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattg

SEQ ID NO: 3 - DNA, 664 bp, Homo sapiens
ctagatctgaattcggtaccctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcg

SEQ ID NO: 4 - protein, 11 aa, Homo sapiens
QAGDVEENPGP

SEQ ID NO: 5 - DNA, 63 bp, Homo sapiens
ggaagcggagctactaacttcagcctgctgaaggctggagacgtggaggagaaccctggacct

SEQ ID NO: 6 - protein, 21 aa, Homo sapiens
GSGATNFSLLQAGDVEENPGP

SEQ ID NO: 7 - DNA, 63 bp, Homo sapiens
agcggagctactaacttcagcctgctgaagcaggctggagacgtggaggagaaccctggacct

SEQ ID NO: 8 - protein, 21 aa, Homo sapiens
SGATNFSLLKQAGDVEENPGP

SEQ ID NO: 9 - protein, 822 aa, Homo sapiens
MSSWIRWHGPAMARLWGFCWLVVGFWRAAFACPTSCKCSASRIWCSDPSPGIVAFPRLEPNSVDPENITEIFIANQKRLEIINEDDVEAYVGLRNLTIVDSGLKFVAHKAFLKNSNLQHINFTRNKLTSLSRKHFRHLDLSELILVGNPFTCSCDIMWIKTLQEAKSSPDTQDLYCLNESSKNIPLANLQIPNCGLPSANLAAPNLTVEEGKSITLSCSVAGDPVPNMYWDVGNLVSKHMNETSHTQGSLRITNISSDDSGKQISCVAENLVGEDQDSVNLTVHFAPTITFLESPTSDHHWCIPFTVKGNPKPALQWFYNGAILNESKYICTKIHVTNHTEYHGCLQLDNPTHMNNGDYTLIAKNEYGKDEKQISAHFMGWPGIDDGANPNYPDVIYEDYGTAANDIGDTTNRSNEIPSTDVTDKTGREHLSVYAVVVIASVVGFCLLVMLFLLKLARHSKFGMKGPASVISNDDDSASPLHHISNGSNTPSSSEGGPDAVIIGMTKIPVIENPQYFGITNSQLKPDTFVQHIKRHNIVLKRELGEGAFGKVFLAECYNLCPEQDKILVAVKTLKDASDNARKDFHREAELLTNLQHEHIVKFYGVCVEGDPLIMVFEYMKHGDLNKFLRAHGPDAVLMAEGNPPTELTQSQMLHIAQQIAAGMVYLASQHFVHRDLATRNCLVGENLLVKIGDFGMSRDVYSTDYYRVGGHTMLPIRWMPPESIMYRKFTTESDVWSLGVVLWEIFTYGKQPWYQLSNNEVIECITQGRVLQRPRTCPQEVYELMLGCWQREPHMRKNIKGIHTLLQNLAKASPVYLDILG

SEQ ID NO: 10 - DNA, 2466 bp, Homo sapiens
atgtcgtcctggataaggtggcatggacccgccatggcgcggctctggggcttctgctggctggttgtgggcttctggagggccgctttcgcctgtcccacgtcctgcaaatgcagtgcctctcggatctggtgcagcgacccttctcctggcatcgtggcatttccgagattggagcctaacagtgtagatcctgagaacatcaccgaaattttcatcgcaaaccagaaaaggttagaaatcatcaacgaagatgatgttgaagcttatgtgggactgagaaatctgacaattgtggattctggattaaaatttgtggctcataaagcatttctgaaaaacagcaacctgcagcacatcaattttacccgaaacaaactgacgagtttgtctaggaaacatttccgtcaccttgacttgtctgaactgatcctggtgggcaatccatttacatgctcctgtgacattatgtggatcaagactctccaagaggctaaatccagtccagacactcaggatttgtactgcctgaatgaaagcagcaagaatattcccctggcaaacctgcagatacccaattgtggtttgccatctgcaaatctggccgcacctaacctcactgtggaggaaggaaagtctatcacattatcctgtagtgtggcaggtgatccggttcctaatatgtattgggatgttggtaacctggtttccaaacatatgaatgaaacaagccacacacagggctccttaaggataactaacatttcatccgatgacagtgggaagcagatctcttgtgtggcggaaaatcttgtaggagaagatcaagattctgtcaacctcactgtgcattttgcaccaactatcacatttctcgaatctccaacctcagaccaccactggtgcattccattcactgtgaaaggcaaccccaaaccagcgcttcagtggttctataacggggcaatattgaatgagtccaaatacatctgtactaaaatacatgttaccaatcacacggagtaccacggctgcctccagctggataatcccactcacatgaacaatggggactacactctaatagccaagaatgagtatgggaaggatgagaaacagatttctgctcacttcatgggctggcctggaattgacgatggtgcaaacccaaattatcctgatgtaatttatgaagattatggaactgcagcgaatgacatcggggacaccacgaacagaagtaatgaaatcccttccacagacgtcactgataaaaccggtcgggaacatctctcggtctatgctgtggtggtgattgcgtctgtggtgggattttgccttttggtaatgctgtttctgcttaagttggcaagacactccaagtttggcatgaaaggcccagcctccgttatcagcaatgatgatgactctgccagcccactccatcacatctccaatgggagtaacactccatcttcttcggaaggtggcccagatgctgtcattattggaatgaccaagatccctgtcattgaaaatccccagtactttggcatcaccaacagtcagctcaagccagacacatttgttcagcacatcaagcgacataacattgttctgaaaagggagctaggcgaaggagcctttggaaaagtgttcctagctgaatgctataacctctgtcctgagcaggacaagatcttggtggcagtgaagaccctgaaggatgccagtgacaatgcacgcaaggacttccaccgtgaggccgagctcctgaccaacctccagcatgagcacatcgtcaagttctatggcgtctgcgtggagggcgaccccctcatcatggtctttgagtacatgaagcatggggacctcaacaagttcctcagggcacacggccctgatgccgtgctgatggctgagggcaacccgcccacggaactgacgcagtcgcagatgctgcatatagcccagcagatcgccgcgggcatggtctacctggcgtcccagcacttcgtgcaccgcgatttggccaccaggaactgcctggtcggggagaacttgctggtgaaaatcggggactttgggatgtcccgggacgtgtacagcactgactactacagggtcggtggccacacaatgctgcccattcgctggatgcctccagagagcatcatgtacaggaaattcacgacggaaagcgacgtctggagcctgggggtcgtgttgtgggagattttcacctatggcaaacagccctggtaccagctgtcaaacaatgaggtgatagagtgtatcactcagggccgagtcctgcagcgaccccgcacgtgcccccaggaggtgtatgagctgatgctggggtgctggcagcgagagccccacatgaggaagaacatcaagggcatccataccctccttcagaacttggccaaggcatctccggtctacctggacattctaggc

SEQ ID NO: 11 - protein, 838 aa, Homo sapiens
MSSWIRWHGPAMARLWGFCWLVVGFWRAAFACPTSCKCSASRIWCSDPSPGIVAFPRLEPNSVDPENITEIFIANQKRLEIINEDDVEAYVGLRNLTIVDSGLKFVAHKAFLKNSNLQHINFTRNKLTSLSRKHFRHLDLSELILVGNPFTCSCDIMWIKTLQEAKSSPDTQDLYCLNESSKNIPLANLQIPNCGLPSANLAAPNLTVEEGKSITLSCSVAGDPVPNMYWDVGNLVSKHMNETSHTQGSLRITNISSDDSGKQISCVAENLVGEDQDSVNLTVHFAPTITFLESPTSDHHWCIPFTVKGNPKPALQWFYNGAILNESKYICTKIHVTNHTEYHGCLQLDNPTHMNNGDYTLIAKNEYGKDEKQISAHFMGWPGIDDGANPNYPDVIYEDYGTAANDIGDTTNRSNEIPSTDVTDKTGREHLSVYAVVVIASVVGFCLLVMLFLLKLARHSKFGMKDFSWFGFGKVKSRQGVGPASVISNDDDSASPLHHISNGSNTPSSSEGGPDAVIIGMTKIPVIENPQYFGITNSQLKPDTFVQHIKRHNIVLKRELGEGAFGKVFLAECYNLCPEQDKILVAVKTLKDASDNARKDFHREAELLTNLQHEHIVKFYGVCVEGDPLIMVFEYMKHGDLNKFLRAHGPDAVLMAEGNPPTELTQSQMLHIAQQIAAGMVYLASQHFVHRDLATRNCLVGENLLVKIGDFGMSRDVYSTDYYRVGGHTMLPIRWMPPESIMYRKFTTESDVWSLGVVLWEIFTYGKQPWYQLSNNEVIECITQGRVLQRPRTCPQEVYELMLGCWQREPHMRKNIKGIHTLLQNLAKASPVYLDILG

SEQ ID NO: 12 - DNA, 2514 bp, Homo sapiens
atgtcgtcctggataaggtggcatggacccgccatggcgcggctctggggcttctgctggctggttgtgggcttctggagggccgctttcgcctgtcccacgtcctgcaaatgcagtgcctctcggatctggtgcagcgacccttctcctggcatcgtggcatttccgagattggagcctaacagtgtagatcctgagaacatcaccgaaattttcatcgcaaaccagaaaaggttagaaatcatcaacgaagatgatgttgaagcttatgtgggactgagaaatctgacaattgtggattctggattaaaatttgtggctcataaagcatttctgaaaaacagcaacctgcagcacatcaattttacccgaaacaaactgacgagtttgtctaggaaacatttccgtcaccttgacttgtctgaactgatcctggtgggcaatccatttacatgctcctgtgacattatgtggatcaagactctccaagaggctaaatccagtccagacactcaggatttgtactgcctgaatgaaagcagcaagaatattcccctggcaaacctgcagatacccaattgtggtttgccatctgcaaatctggccgcacctaacctcactgtggaggaaggaaagtctatcacattatcctgtagtgtggcaggtgatccggttcctaatatgtattgggatgttggtaacctggtttccaaacatatgaatgaaacaagccacacacagggctccttaaggataactaacatttcatccgatgacagtgggaagcagatctcttgtgtggcggaaaatcttgtaggagaagatcaagattctgtcaacctcactgtgcattttgcaccaactatcacatttctcgaatctccaacctcagaccaccactggtgcattccattcactgtgaaaggcaaccccaaaccagcgcttcagtggttctataacggggcaatattgaatgagtccaaatacatctgtactaaaatacatgttaccaatcacacggagtaccacggctgcctccagctggataatcccactcacatgaacaatggggactacactctaatagccaagaatgagtatgggaaggatgagaaacagatttctgctcacttcatgggctggcctggaattgacgatggtgcaaacccaaattatcctgatgtaatttatgaagattatggaactgcagcgaatgacatcggggacaccacgaacagaagtaatgaaatcccttccacagacgtcactgataaaaccggtcgggaacatctctcggtctatgctgtggtggtgattgcgtctgtggtgggattttgccttttggtaatgctgtttctgcttaagttggcaagacactccaagtttggcatgaaagatttctcatggtttggatttgggaaagtaaaatcaagacaaggtgttggcccagcctccgttatcagcaatgatgatgactctgccagcccactccatcacatctccaatgggagtaacactccatcttcttcggaaggtggcccagatgctgtcattattggaatgaccaagatccctgtcattgaaaatccccagtactttggcatcaccaacagtcagctcaagccagacacatttgttcagcacatcaagcgacataacattgttctgaaaagggagctaggcgaaggagcctttggaaaagtgttcctagctgaatgctataacctctgtcctgagcaggacaagatcttggtggcagtgaagaccctgaaggatgccagtgacaatgcacgcaaggacttccaccgtgaggccgagctcctgaccaacctccagcatgagcacatcgtcaagttctatggcgtctgcgtggagggcgaccccctcatcatggtctttgagtacatgaagcatggggacctcaacaagttcctcagggcacacggccctgatgccgtgctgatggctgagggcaacccgcccacggaactgacgcagtcgcagatgctgcatatagcccagcagatcgccgcgggcatggtctacctggcgtcccagcacttcgtgcaccgcgatttggccaccaggaactgcctggtcggggagaacttgctggtgaaaatcggggactttgggatgtcccgggacgtgtacagcactgactactacagggtcggtggccacacaatgctgcccattcgctggatgcctccagagagcatcatgtacaggaaattcacgacggaaagcgacgtctggagcctgggggtcgtgttgtgggagattttcacctatggcaaacagccctggtaccagctgtcaaacaatgaggtgatagagtgtatcactcagggccgagtcctgcagcgaccccgcacgtgcccccaggaggtgtatgagctgatgctggggtgctggcagcgagagccccacatgaggaagaacatcaagggcatccataccctccttcagaacttggccaaggcatctccggtctacctggacattctaggc

SEQ ID NO: 13 - protein, 822 aa, Homo sapiens
MSSWIRWHGPAMARLWGFCWLVVGFWRAAFACPTSCKCSASRIWCSDPSPGIVAFPRLEPNSVDPENITEIFIANQKRLEIINEDDVEAYVGLRNLTIVDSGLKFVAHKAFLKNSNLQHINFTRNKLTSLSRKHFRHLDLSELILVGNPFTCSCDIMWIKTLQEAKSSPDTQDLYCLNESSKNIPLANLQIPNCGLPSANLAAPNLTVEEGKSITLSCSVAGDPVPNMYWDVGNLVSKHMNETSHTQGSLRITNISSDDSGKQISCVAENLVGEDQDSVNLTVHFAPTITFLESPTSDHHWCIPFTVKGNPKPALQWFYNGAILNESKYICTKIHVTNHTEYHGCLQLDNPTHMNNGDYTLIAKNEYGKDEKQISAHFMGWPGIDDGANPNYPDVIYEDYGTAANDIGDTTNRSNEIPSTDVTDKTGREHLSVYAVVVIASVVGFCLLVMLFLLKLARHSKFGMKGPASVISNDDDSASPLHHISNGSNTPSSSEGGPDAVIIGMTKIPVIENPQEFGITNSQLKPDTFVQHIKRHNIVLKRELGEGAFGKVFLAECYNLCPEQDKILVAVKTLKDASDNARKDFHREAELLTNLQHEHIVKFYGVCVEGDPLIMVFEYMKHGDLNKFLRAHGPDAVLMAEGNPPTELTQSQMLHIAQQIAAGMVYLASQHFVHRDLATRNCLVGENLLVKIGDFGMSRDVESTDEERVGGHTMLPIRWMPPESIMYRKFTTESDVWSLGVVLWEIFTYGKQPWYQLSNNEVIECITQGRVLQRPRTCPQEVYELMLGCWQREPHMRKNIKGIHTLLQNLAKASPVELDILG

SEQ ID NO: 14 - DNA, 2466 bp, Homo sapiens
atgtcgtcctggataaggtggcatggacccgccatggcgcggctctggggcttctgctggctggttgtgggcttctggagggccgctttcgcctgtcccacgtcctgcaaatgcagtgcctctcggatctggtgcagcgacccttctcctggcatcgtggcatttccgagattggagcctaacagtgtagatcctgagaacatcaccgaaattttcatcgcaaaccagaaaaggttagaaatcatcaacgaagatgatgttgaagcttatgtgggactgagaaatctgacaattgtggattctggattaaaatttgtggctcataaagcatttctgaaaaacagcaacctgcagcacatcaattttacccgaaacaaactgacgagtttgtctaggaaacatttccgtcaccttgacttgtctgaactgatcctggtgggcaatccatttacatgctcctgtgacattatgtggatcaagactctccaagaggctaaatccagtccagacactcaggatttgtactgcctgaatgaaagcagcaagaatattcccctggcaaacctgcagatacccaattgtggtttgccatctgcaaatctggccgcacctaacctcactgtggaggaaggaaagtctatcacattatcctgtagtgtggcaggtgatccggttcctaatatgtattgggatgttggtaacctggtttccaaacatatgaatgaaacaagccacacacagggctccttaaggataactaacatttcatccgatgacagtgggaagcagatctcttgtgtggcggaaaatcttgtaggagaagatcaagattctgtcaacctcactgtgcattttgcaccaactatcacatttctcgaatctccaacctcagaccaccactggtgcattccattcactgtgaaaggcaaccccaaaccagcgcttcagtggttctataacggggcaatattgaatgagtccaaatacatctgtactaaaatacatgttaccaatcacacggagtaccacggctgcctccagctggataatcccactcacatgaacaatggggactacactctaatagccaagaatgagtatgggaaggatgagaaacagatttctgctcacttcatgggctggcctggaattgacgatggtgcaaacccaaattatcctgatgtaatttatgaagattatggaactgcagcgaatgacatcggggacaccacgaacagaagtaatgaaatcccttccacagacgtcactgataaaaccggtcgggaacatctctcggtctatgctgtggtggtgattgcgtctgtggtgggattttgccttttggtaatgctgtttctgcttaagttggcaagacactccaagtttggcatgaaaggcccagcctccgttatcagcaatgatgatgactctgccagcccactccatcacatctccaatgggagtaacactccatcttcttcggaaggtggcccagatgctgtcattattggaatgaccaagatccctgtcattgaaaatccccaggaatttggcatcaccaacagtcagctcaagccagacacatttgttcagcacatcaagcgacataacattgttctgaaaagggagctaggcgaaggagcctttggaaaagtgttcctagctgaatgctataacctctgtcctgagcaggacaagatcttggtggcagtgaagaccctgaaggatgccagtgacaatgcacgcaaggacttccaccgtgaggccgagctcctgaccaacctccagcatgagcacatcgtcaagttctatggcgtctgcgtggagggcgaccccctcatcatggtctttgagtacatgaagcatggggacctcaacaagttcctcagggcacacggccctgatgccgtgctgatggctgagggcaacccgcccacggaactgacgcagtcgcagatgctgcatatagcccagcagatcgccgcgggcatggtctacctggcgtcccagcacttcgtgcaccgcgatttggccaccaggaactgcctggtcggggagaacttgctggtgaaaatcggggactttgggatgtcccgggacgtggaaagcactgacgaagaaagggtcggtggccacacaatgctgcccattcgctggatgcctccagagagcatcatgtacaggaaattcacgacggaaagcgacgtctggagcctgggggtcgtgttgtgggagattttcacctatggcaaacagccctggtaccagctgtcaaacaatgaggtgatagagtgtatcactcagggccgagtcctgcagcgaccccgcacgtgcccccaggaggtgtatgagctgatgctggggtgctggcagcgagagccccacatgaggaagaacatcaagggcatccataccctccttcagaacttggccaaggcatctccggtcgaactggacattctaggc

SEQ ID NO: 15 - protein, 229 aa, Homo sapiens
APMKEANIRGQGGLAYPGVRTHGTLESVNGPKAGSRGLTSLADTFEHVIEELLDEDQKVRPNEENNKDADLYTSRVMLSSQVPLEPPLLFLLEEYKNYLDAANMSMRVRRHSDPARRGELSVCDSISEWVTAADKKTAVDMSGGTVTVLEKVPVSKGQLKQYFYETKCNPMGYTKEGCRGIDKRHWNSQCRTTQSYVRALTMDSKKRIGWRFIRIDTSCVCTLTIKRGR

SEQ ID NO: 16 - DNA, 690 bp, Homo sapiens
gcccccatgaaagaagcaaacatccgaggacaaggtggcttggcctacccaggtgtgcggacccatgggactctggagagcgtgaatgggcccaaggcaggttcaagaggcttgacatcattggctgacactttcgaacacgtgatagaagagctgttggatgaggaccagaaagttcggcccaatgaagaaaacaataaggacgcagacttgtacacgtccagggtgatgctcagtagtcaagtgcctttggagcctcctcttctctttctgctggaggaatacaaaaattacctagatgctgcaaacatgtccatgagggtccggcgccactctgaccctgcccgccgaggggagctgagcgtgtgtgacagtattagtgagtgggtaacggcggcagacaaaaagactgcagtggacatgtcgggcgggacggtcacagtccttgaaaaggtccctgtatcaaaaggccaactgaagcaatacttctacgagaccaagtgcaatcccatgggttacacaaaagaaggctgcaggggcatagacaaaaggcattggaactcccagtgccgaactacccagtcgtacgtgcgggcccttaccatggatagcaaaaagagaattggctggcgattcataaggatagacacttcttgtgtatgtacattgaccattaaaaggggaagatag

SEQ ID NO: 17 - protein, 229 aa, Homo sapiens
APMKEANIRGQGGLAYPGVRTHGTLESVNGPKAGSRGLTSLADTFEHMIEELLDEDQKVRPNEENNKDADLYTSRVMLSSQVPLEPPLLFLLEEYKNYLDAANMSMRVRRHSDPARRGELSVCDSISEWVTAADKKTAVDMSGGTVTVLEKVPVSKGQLKQYFYETKCNPMGYTKEGCRGIDKRHWNSQCRTTQSYVRALTMDSKKRIGWRFIRIDTSCVCTLTIKRGR

SEQ ID NO: 18 - protein, 119 aa, Homo sapiens
HSDPARRGELSVCDSISEWVTAADKKTAVDMSGGTVTVLEKVPVSKGQLKQYFYETKCNPMGYTKEGCRGIDKRHWNSQCRTTQSYVRALTMDSKKRIGWRFIRIDTSCVCTLTIKRGR

SEQ ID NO: 19 - DNA, 744 bp, Homo sapiens
atgaccatccttttccttactatggttatttcatactttggttgcatgaaggctgcccccatgaaagaagcaaacatccgaggacaaggtggcttggcctacccaggtgtgcggacccatgggactctggagagcgtgaatgggcccaaggcaggttcaagaggcttgacatcattggctgacactttcgaacacgtgatagaagagctgttggatgaggaccagaaagttcggcccaatgaagaaaacaataaggacgcagacttgtacacgtccagggtgatgctcagtagtcaagtgcctttggagcctcctcttctctttctgctggaggaatacaaaaattacctagatgctgcaaacatgtccatgagggtccggcgccactctgaccctgcccgccgaggggagctgagcgtgtgtgacagtattagtgagtgggtaacggcggcagacaaaaagactgcagtggacatgtcgggcgggacggtcacagtccttgaaaaggtccctgtatcaaaaggccaactgaagcaatacttctacgagaccaagtgcaatcccatgggttacacaaaagaaggctgcaggggcatagacaaaaggcattggaactcccagtgccgaactacccagtcgtacgtgcgggcccttaccatggatagcaaaaagagaattggctggcgattcataaggatagacacttcttgtgtatgtacattgaccattaaaaggggaagatag

SEQ ID NO: 20 - protein, 18 aa, Homo sapiens
MTILFLTMVISYFGCMKA

SEQ ID NO: 21 - DNA, 54 bp, Homo sapiens
atgaccatccttttccttactatggttatttcatacttcggttgcatgaaggcg

SEQ ID NO: 22 - protein, 26 aa, Homo sapiens
MFHQVRRVMTILFLTMVISYFGCMKA

SEQ ID NO: 23 - DNA, 78 bp, Homo sapiens
atgttccaccaggtgagaagagtgatgaccatccttttccttactatggttatttcatacttcggttgcatgaaggcg

SEQ ID NO: 24 - protein, 33 aa, Homo sapiens
MQSREEEWFHQVRRVMTILFLTMVISYFGCMKA

SEQ ID NO: 25 - DNA, 99 bp, Homo sapiens
atgcagagccgggaagaggaatggttccaccaggtgagaagagtgatgaccatccttttccttactatggttatttcatacttcggttgcatgaaggcg

SEQ ID NO: 26 - protein, 47 aa, Homo sapiens
MLCAISLCARVRKLRSAGRCGKFHQVRRVMTILFLTMVISYFGCMKA

SEQ ID NO: 27 - DNA, 141 bp, Homo sapiens
atgctctgtgcgatttcattgtgtgctcgcgttcgcaagctccgtagtgcaggaaggtgcgggaagttccaccaggtgagaagagtgatgaccatccttttccttactatggttatttcatacttcggttgcatgaaggcg

SEQ ID NO: 28 - protein, 100 aa, Homo sapiens
MCGATSFLHECTRLILVTTQNAEFLQKGLQVHTCFGVYPHASVWHDCASQKKGCAVYLHVSVEFNKLIPENGFIKFHQVRRVMTILFLTMVISYFGCMKA

SEQ ID NO: 29 - DNA, 300 bp, Homo sapiens
atgtgtggagccaccagttttctccatgagtgcacaaggttaatccttgttactactcagaatgctgagtttctacagaaagggttgcaggtccacacatgttttggcgtctacccacacgcttctgtatggcatgactgtgcatcccagaagaagggctgtgctgtgtacctccacgtttcagtggaatttaacaaactgatccctgaaaatggtttcataaagttccaccaggtgagaagagtgatgaccatccttttccttactatggttatttcatacttcggttgcatgaaggcg

SEQ ID NO: 30 - protein, 20 aa, Homo sapiens
MYRMQLLSCIALSLALVTNS

SEQ ID NO: 31 - DNA, 60 bp, Homo sapiens
atgtacaggatgcaactcctgtcttgcattgcactaagtcttgcacttgtcacaaacagt

SEQ ID NO: 32 - protein, 22 aa, Homo sapiens
MKRRVMIILFLTMVISYFGCMK

SEQ ID NO: 33 - DNA, 70 bp, Homo sapiens
atgaaaagaagagtgatgatcatccttttccttactatggttatttcatacttcggttgcatgaagagcg

SEQ ID NO: 34 - protein, 20 aa, Homo sapiens
MRRMQLLLLIALSLALVTNS

SEQ ID NO: 35 - DNA, 60 bp, Homo sapiens
atgaggaggatgcaactcctgctcctgattgcactaagtcttgcacttgtcacaaacagt

SEQ ID NO: 36 - protein, 21 aa, Homo sapiens
MRRMQLLLLTMVISYFGCMKA

SEQ ID NO: 37 - DNA, 63 bp, Homo sapiens
atgaggaggatgcaactcctgctcctgactatggttatttcatacttcggttgcatgaaggcg

SEQ ID NO: 38 - protein, 18 aa, Homo sapiens
MRILLLTMVISYFGCMKA

SEQ ID NO: 39 - protein, 54 aa, Homo sapiens
ATGAGAATCCTTCTTCTTACTATGGTTATTTCATACTTCGGTTGCATGAAGGCG

SEQ ID NO: 40 - protein, 19 aa, Homo sapiens
MRRILFLTMVISYFGCMKA

SEQ ID NO: 41 - DNA, 57 bp, Homo sapiens
atgagaagaatccttttccttactatggttatttcatacttcggttgcatgaaggcg

SEQ ID NO: 42 - protein, 18 aa, Homo sapiens
MRRFLFLLVISYFGCMKA

SEQ ID NO: 43 - DNA, 54 bp, Homo sapiens
atgaggaggttccttttccttcttgttatttcatacttcggttgcatgaaggcg

SEQ ID NO: 44 - protein, 15 aa, Homo sapiens
MRRFLFLLYFGCMKA

SEQ ID NO: 45 - DNA, 45 bp, Homo sapiens
atgaggaggttccttttccttctttacttcggttgcatgaaggcg

SEQ ID NO: 46 - DNA, 130 bp, Homo sapiens
cctgcaggcagctgcgcgctcgctcgctcactgaggccgcccgggcgtcgggcgacctttggtcgcccggcctcagtgagcgagcgagcgcgcagagagggagtggccaactccatcactaggggttcct

SEQ ID NO: 47 - DNA, 141 bp, Homo sapiens
aggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 48 - DNA, 584 bp, Homo sapiens
gcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcg

SEQ ID NO: 49 - protein, 210 aa, Homo sapiens
MLPLPSCSLPILLLFLLPSVPIESQPPPSTLPPFLAPEWDLLSPRVVLSRGAPAGPPLLFLLEAGAFRESAGAPANRSRRGVSETAPASRRGELAVCDAVSGWVTDRRTAVDLRGREVEVLGEVPAAGGSPLRQYFFETRCKADNAEEGGPGAGGGGCRGVDRRHWVSECKAKQSYVRALTADAQGRVGWRWIRIDTACVCTLLSRTGRA

SEQ ID NO: 50 - DNA, 630 bp, Homo sapiens
atgctccctctcccctcatgctccctccccatcctcctccttttcctcctccccagtgtgccaattgagtcccaacccccaccctcaacattgcccccttttctggcccctgagtgggaccttctctccccccgagtagtcctgtctaggggtgcccctgctgggccccctctgctcttcctgctggaggctggggcctttcgggagtcagcaggtgccccggccaaccgcagccggcgtggggtgagcgaaactgcaccagcgagtcgtcggggtgagctggctgtgtgcgatgcagtcagtggctgggtgacagaccgccggaccgctgtggacttgcgtgggcgcgaggtggaggtgttgggcgaggtgcctgcagctggcggcagtcccctccgccagtacttctttgaaacccgctgcaaggctgataacgctgaggaaggtggcccgggggcaggtggagggggctgccggggagtggacaggaggcactgggtatctgagtgcaaggccaagcagtcctatgtgcgggcattgaccgctgatgcccagggccgtgtgggctggcgatggattcgaattgacactgcctgcgtctgcacactcctcagccggactggccgggcc

SEQ ID NO: 51 - protein, 24 aa, Homo sapiens
MLPLPSCSLPILLLFLLPSVPIES

SEQ ID NO: 52 - DNA, 72 bp, Homo sapiens
atgctccctctcccctcatgctccctccccatcctcctccttttcctcctccccagtgtgccaattgagtcc

SEQ ID NO: 53 - protein, 56 aa, Homo sapiens
QPPPSTLPPFLAPEWDLLSPRVVLSRGAPAGPPLLFLLEAGAFRESAGAPANRSRR

SEQ ID NO: 54 - DNA, 168 bp, Homo sapiens
caacccccaccctcaacattgcccccttttctggcccctgagtgggaccttctctccccccgagtagtcctgtctaggggtgcccctgctgggccccctctgctcttcctgctggaggctggggcctttcgggagtcagcaggtgccccggccaaccgcagccggcgt

SEQ ID NO: 55 - protein, 130 aa, Homo sapiens
GVSETAPASRRGELAVCDAVSGWVTDRRTAVDLRGREVEVLGEVPAAGGSPLRQYFFETRCKADNAEEGGPGAGGGGCRGVDRRHWVSECKAKQSYVRALTADAQGRVGWRWIRIDTACVCTLLSRTGRA

SEQ ID NO: 56 - DNA, 390 bp, Homo sapiens
ggggtgagcgaaactgcaccagcgagtcgtcggggtgagctggctgtgtgcgatgcagtcagtggctgggtgacagaccgccggaccgctgtggacttgcgtgggcgcgaggtggaggtgttgggcgaggtgcctgcagctggcggcagtcccctccgccagtacttctttgaaacccgctgcaaggctgataacgctgaggaaggtggcccgggggcaggtggagggggctgccggggagtggacaggaggcactgggtatctgagtgcaaggccaagcagtcctatgtgcgggcattgaccgctgatgcccagggccgtgtgggctggcgatggattcgaattgacactgcctgcgtctgcacactcctcagccggactggccgggcc

SEQ ID NO: 57 - DNA, 592 bp, Homo sapiens
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctg

SEQ ID NO: 58 - DNA, 247 bp, Homo sapiens
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgt

SEQ ID NO: 59 - DNA, 224 bp, Homo sapiens
agcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggta

SEQ ID NO: 60 - protein, 18 aa, Homo sapiens
MTILFLTMVISYFGCMKA

SEQ ID NO: 61 - DNA, 54 bp, Homo sapiens; identical to SEQ ID NO: 21

SEQ ID NO: 62 - protein, 2 aa, Homo sapiens
MR

SEQ ID NO: 63 - DNA, 6 bp, Homo sapiens
atgaga

SEQ ID NO: 64 - protein, 3 aa, Homo sapiens
MRR

SEQ ID NO: 65 - DNA, 9 bp, Homo sapiens
atgagaaga

SEQ ID NO: 66 - protein, 4 aa, Homo sapiens
MRRR

SEQ ID NO: 67 - DNA, 12 bp, Homo sapiens
atgagaagaaga

SEQ ID NO: 68 - protein, 2 aa, Homo sapiens
MK

SEQ ID NO: 69 - DNA, 6 bp, Homo sapiens
atgaaa

SEQ ID NO: 70 - protein, 3 aa, Homo sapiens
MKK

SEQ ID NO: 71 - DNA, 9 bp, Homo sapiens
atgaaaaka

SEQ ID NO: 72 - protein, 4 aa, Homo sapiens
MKKK

SEQ ID NO: 73 - DNA, 12 bp, Homo sapiens
atgaaaaaaaaa

SEQ ID NO: 74 - protein, 4 aa, Homo sapiens
MKRR

SEQ ID NO: 75 - DNA, 12 bp, Homo sapiens
atgaaaagaaga

SEQ ID NO: 76 - protein, 4 aa, Homo sapiens
MRKR

SEQ ID NO: 77 - DNA, 12 bp, Homo sapiens
atgagaaaaaga

SEQ ID NO: 78 - protein, 4 aa, Homo sapiens
MRRK

SEQ ID NO: 79 - DNA, 12 bp, Homo sapiens
atgagaagaaaa

SEQ ID NO: 80 - protein, 4 aa, Homo sapiens
MKKR

SEQ ID NO: 81 - DNA, 12 bp, Homo sapiens
atgaaaaaaaga

SEQ ID NO: 82 - protein, 4 aa, Homo sapiens
FLFL

SEQ ID NO: 83 - DNA, 12 bp, Homo sapiens
ttccttttcctt

SEQ ID NO: 84 - protein, 4 aa, Homo sapiens
FFFL

SEQ ID NO: 85 - DNA, 12 bp, Homo sapiens
ttcttcttcctt

SEQ ID NO: 86 - protein, 4 aa, Homo sapiens
FIFL

SEQ ID NO: 87 - DNA, 12 bp, Homo sapiens
ttcatcttcctt

SEQ ID NO: 88 - protein, 4 aa, Homo sapiens
FIFI

SEQ ID NO: 89 - DNA, 12 bp, Homo sapiens
ttcatcttcatc

SEQ ID NO: 90 - protein, 4 aa, Homo sapiens
FVFI

SEQ ID NO: 91 - DNA, 12 bp, Homo sapiens
ttcgttttcatc

SEQ ID NO: 92 - protein, 4 aa, Homo sapiens
FVFV

SEQ ID NO: 93 - DNA, 12 bp, Homo sapiens
ttcgttttcgtt

SEQ ID NO: 94 - protein, 4 aa, Homo sapiens
FLFV

SEQ ID NO: 95 - DNA, 12 bp, Homo sapiens
ttccttttcgtt

SEQ ID NO: 96 - protein, 4 aa, Homo sapiens
FIFV

SEQ ID NO: 97 - DNA, 12 bp, Homo sapiens
ttcatcttcgtt

SEQ ID NO: 98 - protein, 4 aa, Homo sapiens
FFFI

SEQ ID NO: 99 - DNA, 12 bp, Homo sapiens
ttcttcttcatc

SEQ ID NO: 100 - protein, 4 aa, Homo sapiens
FFFV

SEQ ID NO: 101 - DNA, 12 bp, Homo sapiens
ttcttcttcgtt

SEQ ID NO: 102 - protein, 5 aa, Homo sapiens
FILFL

SEQ ID NO: 103 - DNA, 15 bp, Homo sapiens
ttcatccttttcctt

SEQ ID NO: 104 - DNA, 1203 bp, Homo sapiens
atgactatcctgtttctgacaatggttattagctatttcggttgcatgaaggctcacagtgatcccgcacgccgcggagaacttagcgtgtgcgacagcatcagcgagtgggtcaccgccgccgataagaagaccgctgtggatatgtccggcgggaccgtcactgtactcgaaaaagttccagtgagcaaaggccaactgaaacaatatttctatgaaactaagtgcaaccccatggggtacaccaaggagggctgccggggaatcgacaagagacactggaattcccagtgccggaccactcagagctacgtccgcgccttgacgatggattcaaagaagcgcatcggatggcggttcataagaatcgacaccagttgtgtgtgcacgctgacgataaaacgggggcgggcccccgtgaagcagaccctgaactttgatttgctcaagttggcgggggatgtggaaagcaatcccgggccaatggtgagcaagggcgaggagctgttcaccggcgttgtgccaatactggttgagttggatggcgatgtcaacggacacaaatttagcgtaagcggggagggagagggcgacgccacatatggcaagctgaccctgaagttcatttgcacgaccggcaaattgcccgtcccttggcccacacttgtgacgaccctgacttatggcgtacagtgcttcagcaggtaccctgatcatatgaagcaacacgacttctttaagagtgccatgccagagggatacgtccaggaaagaaccatattcttcaaagatgatggaaattacaaaacccgggcagaggtcaagtttgaaggcgacaccctggtgaacaggatcgaactcaaaggcatcgatttcaaagaggacggaaacatcctcggacacaaactggaatacaattacaacagccacaacgtctacatcatggcagataaacaaaagaacggtattaaagtgaacttcaagatccggcacaacatcgaagacggctccgtccagcttgccgaccactaccagcaaaataccccgatcggcgacggccccgttctcctccccgataatcactacctgagtacacagtcagccttgagcaaagaccctaatgaaaagcgggaccacatggttttgctggagttcgttaccgcagcgggtattacgctgggtatggacgagctttacaagtaa

SEQ ID NO: 105 - DNA, 1203 bp, Homo sapiens
atgactatcctgtttctgacaatggttattagctatttcggttgcatgaaggctcacagtgatcccgcacgccgcggagaacttagcgtgtgcgacagcatcagcgagtgggtcaccgccgccgataagaagaccgctgtggatatgtccggcgggaccgtcactgtactcgaaaaagttccagtgagcaaaggccaactgaaacaatatttctatgaaactaagtgcaaccccatggggtacaccaaggagggctgccggggaatcgacaagagacactggaattcccagtgccggaccactcagagctacgtccgcgccttgacgatggattcaaagaagcgcatcggatggcggttcataagaatcgacaccagttgtgtgtgcacgctgacgataaaacgggggcgggcccctgtcaaacaaaccctcaattttgacttgctgaagcttgctggggatgtcgagtccgctgccgcggctatggtgagcaagggcgaggagctgttcaccggcgttgtgccaatactggttgagttggatggcgatgtcaacggacacaaatttagcgtaagcggggagggagagggcgacgccacatatggcaagctgaccctgaagttcatttgcacgaccggcaaattgcccgtcccttggcccacacttgtgacgaccctgacttatggcgtacagtgcttcagcaggtaccctgatcatatgaagcaacacgacttctttaagagtgccatgccagagggatacgtccaggaaagaaccatattcttcaaagatgatggaaattacaaaacccgggcagaggtcaagtttgaaggcgacaccctggtgaacaggatcgaactcaaaggcatcgatttcaaagaggacggaaacatcctcggacacaaactggaatacaattacaacagccacaacgtctacatcatggcagataaacaaaagaacggtattaaagtgaacttcaagatccggcacaacatcgaagacggctccgtccagcttgccgaccactaccagcaaaataccccgatcggcgacggccccgttctcctccccgataatcactacctgagtacacagtcagccttgagcaaagaccctaatgaaaagcgggaccacatggttttgctggagttcgttaccgcagcgggtattacgctgggtatggacgagctttacaagtaa

SEQ ID NO: 106 - DNA, 1203 bp, Homo sapiens
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaggaccccaacgagaagcgcgatcacatggtcctgctggagttcgtgaccgccgccgggatcactctcggcatggacgagctgtacaaggctcccgttaaacaaactctgaacttcgacctgctgaagctggctggagacgtggagtccaaccctggacctatgaccatccttttccttactatggttatttcatacttcggttgcatgaaggcgcactccgaccctgcccgccgtggggagctgagcgtgtgtgacagtattagcgagtgggtcacagcggcagataaaaagactgcagtggacatgtctggcgggacggtcacagtcctagagaaagtcccggtatccaaaggccaactgaagcagtatttctacgagaccaagtgtaatcccatgggttacaccaaggaaggctgcaggggcatagacaaaaggcactggaactcgcaatgccgaactacccaatcgtatgttcgggcccttactatggatagcaaaaagagaattggctggcgattcataaggatagacacttcctgtgtatgtacactgaccattaaaaggggaagatag

SEQ ID NO: 107 - DNA, 2940 bp, Homo sapiens
atgagcccatggctgaagtggcacggaccagcaatggcaagactgtggggcctgtgcctgctggtgctgggcttctggagagccagcctggcctgtccaacctcctgcaagtgtagctccgccaggatctggtgcacagagccttctccaggcatcgtggcctttccccgcctggagcctaacagcgtggatcccgagaatatcaccgagatcctgatcgccaaccagaagcggctggagatcatcaatgaggacgatgtggaggcctacgtgggcctgagaaacctgacaatcgtggactccggcctgaagttcgtggcctataaggcctttctgaagaactctaatctgaggcacatcaacttcacccgcaataagctgacatctctgagccggagacactttcggcacctggatctgtccgacctgatcctgaccggcaatccattcacatgctcttgtgacatcatgtggctgaagaccctgcaggagacaaagtctagccccgatacccaggacctgtactgtctgaacgagtcctctaagaatatgcctctggccaacctgcagatccctaattgtggactgccaagcgcccggctggccgcacctaacctgacagtggaggagggcaagtccgtgacactgtcctgttctgtgggcggcgatcccctgcctaccctgtattgggacgtgggcaacctggtgtctaagcacatgaatgagacctcccacacacagggctctctgagaatcacaaatatcagctccgacgatagcggcaagcagatctcttgcgtggcagagaacctggtgggagaggatcaggacagcgtgaatctgaccgtgcacttcgcccccaccatcacatttctggagtctcctaccagcgatcaccactggtgcatccccttcacagtgcggggaaacccaaagcccgccctgcagtggttttacaacggcgccatcctgaatgagtccaagtatatctgtaccaagatccacgtgaccaaccacacagagtaccacggctgcctgcagctggataatcccacccacatgaacaatggcgactacacactgatggccaagaacgagtatggcaaggacgagaggcagatcagcgcccacttcatgggccgccctggagtggattatgagaccaaccctaattacccagaggtgctgtatgaggactggaccacacctaccgatatcggcgacaccacaaacaagtctaatgagatcccaagcacagatgtggccgaccagtctaacagggagcacctgagcgtgtacgcagtggtggtcatcgcctccgtggtgggcttctgcctgctggtcatgctgctgctgctgaagctggcccgccactctaagtttggcatgaagggcccagcctccgtgatctctaatgacgatgacagcgccagccccctgcaccacatcagcaacggctccaataccccttctagctccgagggcggcccagatgccgtgatcatcggcatgacaaagatccccgtgatcgagaaccctcagtacttcggcatcaccaattcccagctgaagcctgacacatttgtgcagcacatcaagcggcacaacatcgtgctgaagagggaactgggagagggagccttcggcaaggtgtttctggccgagtgctataacctgtgcccagagcaggataagatcctggtggccgtgaagaccctgaaggatgccagcgacaacgcccggaaggacttccacagagaggccgagctgctgacaaatctgcagcacgagcacatcgtgaagttttacggcgtgtgcgtggagggcgaccctctgatcatggtgttcgagtatatgaagcacggcgatctgaacaagtttctgagagcacacggaccagatgccgtgctgatggcagagggaaatccccctaccgagctgacacagtctcagatgctgcacattgcacagcagattgcagcaggaatggtgtacctggccagccagcacttcgtgcacagggatctggcaaccagaaactgcctggtgggagagaatctgctggtgaagatcggcgactttggcatgtcccgggacgtgtactctaccgactactatagagtgggcggccacacaatgctgcccatcaggtggatgccacccgagagcatcatgtatcgcaagttcaccacagagtctgacgtgtggagcctgggcgtggtgctgtgggagatctttacctacggcaagcagccttggtatcagctgtccaacaatgaagtgatcgagtgtattacacagggacgcgtgctgcagaggccacgcacatgcccccaggaggtgtacgagctgatgctgggctgttggcagcgggagccacacaccagaaagaacatcaagagcatccacacactgctgcagaatctggccaaggcctcccccgtgtatctggacatcctgggcagcggagctactaacttcagcctgctgaagcaggctggagacgtggaggagaaccctggacctatgagaatccttcttcttactatggttatttcatacttcggttgcatgaaggcgcactccgaccctgcccgccgtggggagctgagcgtgtgtgacagtattagcgagtgggtcacagcggcagataaaaagactgcagtggacatgtctggcgggacggtcacagtcctagagaaagtcccggtatccaaaggccaactgaagcagtatttctacgagaccaagtgtaatcccatgggttacaccaaggaaggctgcaggggcatagacaaaaggcactggaactcgcaatgccgaactacccaatcgtatgttcgggcccttactatggatagcaaaaagagaattggctggcgattcataaggatagacacttcctgtgtatgtacactgaccattaaaaggggaagatag

SEQ ID NO: 108 - DNA, 2943 bp, Homo sapiens
atgtcatcttggatccgctggcacgggccagcgatggcccgattgtggggcttctgctggcttgttgtaggcttctggcgcgcggcgttcgcgtgtccgacctcttgcaaatgctcagcaagccgaatttggtgctcagaccctagtccaggaattgttgcattcccccgactggaaccaaactccgtcgacccggagaatataactgagatatttattgcaaatcaaaaacgccttgaaatcattaacgaggatgacgtggaggcctacgttggtttgagaaatcttactattgtcgactccggacttaaatttgtagctcataaagccttcctgaagaactctaatctgcagcacattaatttcacgagaaataagctgaccagcttgtcccggaagcatttccgccatctcgacctgagcgagctcatactggtcggaaacccatttacgtgctcctgtgacatcatgtggatcaaaactctgcaagaggcgaaaagtagtccggatacccaagacctttactgtcttaatgaaagctcaaaaaatatcccgctggccaacctgcagataccgaactgcggacttcctagtgcgaatttggctgccccaaatcttaccgtcgaagaaggcaaatcaatcacgctttcttgttctgtagctggagatccagtgcctaatatgtattgggacgtgggtaacctcgtctcaaaacatatgaacgaaacgagccacacccagggctctttgcggataacaaacatctcctctgatgattctggaaagcaaatcagttgcgtagctgaaaatctggttggcgaagatcaagattcagtcaatctgacagtccatttcgccccaacgatcacctttctggagagcccaactagcgatcaccactggtgtattccgtttacggtaaaaggaaatccaaaacctgcactccaatggttttataatggagccatcttgaatgaaagcaaatatatctgtactaaaatccatgtgacgaatcacaccgagtatcacgggtgtcttcaattggataatccaacccatatgaataatggtgattatactttgatagcgaagaacgaatacggcaaagacgaaaagcaaatatccgcacatttcatgggttggcctggcatcgacgacggtgcgaacccgaactacccagatgttatttacgaggattatgggactgcggcaaacgacattggcgacaccacaaaccgaagcaacgagataccaagtactgacgtcactgacaaaacgggtcgagagcatttgtctgtttacgccgttgttgttatcgcctcagttgtcggattttgcctgttggtcatgcttttcctcctgaagctcgcgcgacattccaagtttggcatgaaggggccagcaagtgttatatccaatgatgatgatagcgcttctccattgcaccacataagtaacggctcaaacacgccgtcatctagtgaaggtggaccagacgcggtcattatagggatgactaaaattcccgtaatcgaaaaccctcagtacttcggcataaccaacagtcagcttaaacccgatactttcgtgcagcacatcaaaaggcacaacatagtcctcaagcgcgaactcggggagggagccttcggaaaggtctttcttgctgagtgctataatttgtgtcctgagcaggataaaattcttgtggctgtaaaaactctcaaagatgcttccgacaacgcacggaaggattttcatcgggaggccgaactgttgacgaatttgcagcacgagcatatagtaaagttctacggggtatgtgttgagggggacccgttgattatggtcttcgagtatatgaagcacggggacctgaacaaatttttgcgcgcccatgggcctgatgccgtccttatggcagaagggaaccctccaacagaactcacccagagtcagatgttgcacatagcgcaacagatcgcggccggcatggtttacctggccagtcaacacttcgtgcatagagatcttgccactcgcaactgtttggtcggggagaaccttctggttaagattggtgactttggtatgtcacgagatgtgtattccactgactattacagagttgggggtcatacaatgcttcctattcggtggatgccccccgaatccatcatgtacagaaagttcacgacagagagtgatgtttggagtctcggcgtggtgctctgggaaattttcacatacggaaagcagccgtggtatcaacttagcaacaatgaggtgatagagtgtattacacagggtcgggtgttgcagcgccctcgaacgtgcccacaagaagtatatgaacttatgctcgggtgctggcaaagagaaccacatatgagaaaaaatatcaaggggatacatacattgcttcagaacttggccaaggcatcacccgtctacctcgatatactgggcagcggagctactaacttcagcctgctgaagcaggctggagacgtggaggagaaccctggacctatgagaatccttcttcttactatggttatttcatacttcggttgcatgaaggcgcactccgaccctgcccgccgtggggagctgagcgtgtgtgacagtattagcgagtgggtcacagcggcagataaaaagactgcagtggacatgtctggcgggacggtcacagtcctagagaaagtcccggtatccaaaggccaactgaagcagtatttctacgagaccaagtgtaatcccatgggttacaccaaggaaggctgcaggggcatagacaaaaggcactggaactcgcaatgccgaactacccaatcgtatgttcgggcccttactatggatagcaaaaagagaattggctggcgattcataaggatagacacttcctgtgtatgtacactgaccattaaaaggggaagatag

(19) United States
(12) Patent Application Publication
(10) Pub. No.: US 2020/0046850 A1
(43) Pub. Date: Feb. 13, 2020

(54) Genetic Construct

(71) Applicant: Quethera Limited, Canterbury (GB)

(72) Inventors: Peter WIDDOWSON, Canterbury (GB); Keith MARTIN, Canterbury (GB)

(21) Appl. No.: 16/497,412

(22) PCT Filed: Mar. 28, 2018
(86) PCT No.: PCT/GB2018/050824

§ 371 Date: Sep. 24, 2019

(30) Foreign Application Priority Data
Apr. 5, 2017 (GB) 1705484.2

(51) Int. Cl. A61K 48/00; A61K 38/17; A61K 38/18; C12N 15/86; C12N 7/00; A61P 25/28

(52) U.S. Cl. CPC A61K 48/005; A61K 38/177; A61K 38/185; C12N 2750/14143; C12N 7/00; A61P 25/28; C12N 15/86

(57) ABSTRACT

The invention provides genetic constructs and recombinant vectors comprising such constructs. The constructs and vectors can be used in gene therapy methods for the treatment, prevention or amelioration of a neurodegenerative disorder, including Alzheimer's disease, Parkinson's disease, Huntington's disease, motor neurone disease, or for the treatment of stroke, or for promoting nerve regeneration and/or survival.

The present invention relates to genetic constructs, and in particular to recombinant vectors comprising such constructs, and to the uses of the constructs and vectors in gene therapy methods for the treatment, prevention or amelioration of a neurodegenerative disorder, or for the treatment of stroke, or for promoting nerve regeneration and/or survival.

Neurodegenerative diseases are those that primarily affect neurons. The degenerative process can involve the progressive loss of neuronal structure, the progressive loss of neuronal function, or progressive neuron cell death. Many specific disorders are categorised as neurodegenerative diseases. Parkinson's disease is a long-term neurodegenerative disorder, and has been estimated to affect approximately seven million people. Huntington's disease is also a long-term neurodegenerative disorder, and so there is a need for improved treatments for Parkinson's disease and Huntington's disease, and the promotion of nerve regeneration or survival could be beneficial to such patients.

Motor neurone disease includes any disorder that has a neurodegenerative effect on motor neurons. This includes amyotrophic lateral sclerosis (ALS), primary lateral sclerosis (PLS), progressive muscular atrophy (PMA), progressive bulbar palsy (PBP), pseudobulbar palsy, or spinal muscular atrophies. Stroke occurs when blood flow to the brain is interrupted or reduced, and the poor blood flow can result in cell death.

Alzheimer's disease accounts for about 60% of all dementias, and estimates are that over 26 million people worldwide are reported to have Alzheimer's disease [1]. Dementia involves a progressive decline in mental function, usually including deficits in memory, language and cognitive processes. Alzheimer's disease can not only affect patients themselves, but has a significant impact on the millions of carers, often unpaid, who are needed to look after them. Since the greatest risk factor of Alzheimer's disease is age, there is a dramatic increase in the prevalence as people survive longer in old-age [1]. Increasing numbers of Alzheimer patients is already having major impacts on global healthcare systems. Typical pathology associated with Alzheimer's disease involves gross atrophy of the brain, thinning of the grey matter in the cerebral cortex, enlarged ventricles indicative of neuronal loss, microscopic extracellular amyloid plaques comprising beta-amyloid peptide [Aβ], which aggregate into protein clumps, intracellular neurofibrillary tangles comprising aggregated Tau protein, and cerebrovascular amyloid, i.e. amyloid protein surrounding the blood vessels. In Alzheimer's disease, many areas in the brain have amyloid plaques caused by extracellular deposits of misfolded amyloid β-peptide, and neurofibrillary tangles composed of hyperphosphorylated Tau protein, especially the frontal, temporal and parietal cortices, the hippocampus, and the cholinergic nuclei of the basal forebrain. These brain regions represent key areas involved in the neuronal circuitry essential for short-term memory. Amyloid plaque deposition appears randomly throughout the brain, whereas the appearance of intracellular neurofibrillary tangles seems to follow a well-defined pattern [2] being detected first in the trans-entorhinal cortex. The neurofibrillary tangles are then observed to spread sequentially to the entorhinal cortex, to areas of the hippocampus and then outwards to the cerebral cortex. Numerous studies have indicated that one of the earliest changes in Alzheimer's disease involves the loss of synapses, which correlates with mental decline [3] eventually leading to marked cell loss throughout a number of brain areas. The symptoms of the disease therefore follow the slow progression of destruction throughout the brain, beginning with the inability to make new memories, a process which is dependent on the hippocampus.

Brain-derived neurotrophic factor (BDNF) along with nerve growth factor (NGF), neurotrophin-3 (NT-3), and neurotrophin-4/5 (NT-4/5) are members of the neurotrophin family of trophic factors [4-5]. The neurotrophins play essential roles in the development, survival and function of a wide range of neurons in both the peripheral and central nervous systems. Neurotrophins interact with two cell surface receptors, low affinity p75^{NTR} receptors and the high affinity tyrosine receptor kinase (Trk) family [4-5]. Nerve growth factor (NGF) preferentially binds TrkA, Brain Derived Neurotrophic Factor (BDNF) and Neurotrophin-4/5 (NT4/5) bind to tropmyosin receptor kinase-B (TrkB), and Neurotrophin-3 (NT-3) binds TrkC (and TrkA to a lesser extent) [12-13].

Brain-derived neurotrophic factor (BDNF) is a protein which is highly expressed and widely distributed throughout the central nervous system, especially in the hippocampus and cerebral cortex [6-7]. It has been shown to be important in the survival and function of hippocampal, cortical, cholinergic and dopaminergic neurons [8]. BDNF is associated with a number of disorders of the brain, including Alzheimer's disease, Huntington's disease, depression, schizophrenia, and Rett syndrome. It has been hypothesised that early memory dysfunction seen in Alzheimer's disease may be related to the levels of BDNF in the hippocampus as there are reports of substantial reduced BDNF mRNA levels in Alzheimer's disease hippocampus [9] and parietal cortex [10] and decreased protein levels of BDNF in entorhinal cortex, hippocampus, temporal, frontal and parietal cortex [11-16]. Changes in BDNF levels, however, seem to be due to specific downregulation of certain BDNF transcripts. Meta-analysis also shows a significant decrease of neurotrophin levels in blood of Alzheimer patients compared to healthy subjects [17]. Moreover lower cerebrospinal fluid concentration of BDNF was shown to predict progression from mild cognitive impairment (MCI) to Alzheimer's disease [18].

A number of studies indicate that subjects exhibiting the Val66Met polymorphism (where the valine is substituted by methionine) of the pro-domain of BDNF is associated with increased progression to Alzheimer's disease [19] and other BDNF polymorphisms may also be implicated. Loss of proBDNF a larger precursor version of BDNF and of mature BDNF (mBDNF) occurs early in the disease (before plaque deposition) and correlates with memory deficits [20-21]. These data strongly suggest a link between reduced BDNF concentrations, synaptic loss and cell dysfunction which underlie Alzheimer's cognitive impairment. BDNF has also been shown to induce rapid Tau dephosphorylation in neuronal cells through interactions with the TrkB receptor and subsequent increase in phosphoinositol-3-kinase (PI3K) and protein kinase (Akt) signalling, [22-23]. Therefore, decreases in BDNF concentrations might also contribute to Tau hyperphosphorylation, a pathological hallmark of AD. There also appears to be converse effect with increased Tau causing a reduction in BDNF expression in mice [24]. Recent data has also demonstrated potential exacerbation in Aβ neurotoxicity in the presence of pro-domain of neurotrophins, including BDNF [25].

Changes in neurons expressing the mBDNF receptor TrkB, have also been found in post-mortem Alzheimer brains. For example, a 47% reduction in TrkB positive neurons has been reported in post-mortem brains from Alzheimer's sufferers [26]. This may be attributed either to a loss of neurons which normally express the receptor or to a biochemical down-regulation of TrkB expression. The decrease of TrkB could also be aggravated by the up-regulation of truncated receptor isoforms TrkB-T1 and TrkB-Shc in both frontal and temporal cortex in Alzheimer's disease which do not display kinase activity essential for neuronal survival [27]. Activation of the protease, calpain, by Aβ in neuronal cultures induces a decrease of TrkB [28] by cleavage near the receptor She docking site leading to the conversion of fully functional receptors into truncated isoform with defective kinase activity. The effect of conversion of functional TrkB receptors into truncated isoform may then act as a neurotrophin sink or dominant negative receptor. In a mouse model of Alzheimer's disease, knockout of the TrkB receptors was observed to exacerbate Alzheimer's disease-like signalling aberrations and memory deficits without affecting the deposition of Aβ [29]. These data suggest that loss in TrkB receptors and/or loss in activity through reduced BDNF production and secretion represent important elements in producing Alzheimer-like symptoms and pathophysiology.

Other important mechanisms contributing to the deficiency of BDNF/TrkB signalling in Alzheimer's brains includes suppression of mitogen activated protein kinase (MAPK/ERK) and PI3K/Akt pathways by sub-lethal concentrations of Aβ, without interference of TrkB-FL and phospholipase-γ (PLCγ) activation [30], and the disruption of BDNF-induced TrkB endocytosis. The exposure to Aβ oligomers can impair receptor endocytosis and downstream Akt activation through glycogen synthase kinase-3β (GSK3β)-mediated dynamin-1 phosphorylation [31]. In addition, the Aβ oligomers have been shown to interfere with BDNF-mediated TrkB retrograde trafficking [32] through disruption of the ubiquitin system [33] and altering calcium homeostasis [34].

The overall picture is for significant impairment of neurotrophic signalling in Alzheimer's disease, and in particular for the BDNF system. Supplementation or boosting BDNF signalling has been examined in several animal models of Alzheimer's disease. For example, injections of BDNF ameliorate learning deficits in a rat model of Alzheimer's disease induced by Aβ[1-42] [35]. Injections of a novel fusion peptide containing the active domain of BDNF with an HIV-encoded transactivator of transcription (TAT) that can penetrate the brain significantly improved spatial memory with activation of the TrkB/ERK1/2/Akt pathway and restoration of several memory-associated proteins in animal models [36]. In addition, expression of BDNF using lentiviral-based gene therapy was shown to have a neuroprotective effect in mouse transgenic models of Alzheimer's disease and in older primates which are showing cognitive decline [37].

BDNF may be produced in the brain and may be transported to the periphery, where it can support neurons and maintain their survival [38-44]. In certain conditions, such as during excitotoxic insults with glutamate receptor agonists, such as N-methyl-D-aspartate, BDNF can also be produced in peripheral neurons although at relatively low levels [45-46]. BDNF is normally produced as a prepro-polypeptide (i.e. preproBDNF) containing a short signal peptide sequence, which facilitates trafficking of the entire polypeptide to vesicles for release into the extracellular space. Cleavage and removal of the signal peptide converts preproBDNF into proBDNF. An N-terminal proBDNF sequence is then cleaved either intracellulary or extracellularly to create mature BDNF (mBDNF) [47]. Both pro-BDNF and mBDNF possess biological activity with pro-BDNF preferentially activating p75^{NTR} receptors and the shorter mBDNF activating TrkB receptors [48-50]. Activation of p75^{NTR} and TrkB receptors in the retina, for instance, show opposing effects on retinal ganglion cell (RGC) survival, the former being responsible for apoptosis through direct RGC-cell-body-p75^{R}-activation [48-51] or indirectly via p75^{NTR} activation on Miller cells, thereby stimulating release of Tumour Necrosis Factor-alpha (TNF-α) which further promotes RGC loss [52].

Animal models of glaucoma have demonstrated that following nerve crush, or raised IOP, there is a shift away from neurotrophic mBDNF/TrkB signalling towards pro-BDNF/p75^{NTR} pathways. Reduced levels of mBDNF and TrkB receptors in the retina have been demonstrated [50, 53-54] together with opposing elevations in the relative levels of pro-BDNF [28] and p75^{NTR} receptors [55]. Supplementation of mBDNF through ocular injections of recombinant protein to rats with experimentally elevated IOP increases the survival of RGCs compared with untreated eyes, thereby confirming a key neuroprotective role for this neurotrophin [42-44].

In view of the above, there is therefore a need for an improved gene therapy for the promotion of nerve regeneration or survival, for the treatment, prevention, or amelioration of a neurodegenerative disorder or stroke.

The inventors have constructed a novel genetic construct, which encodes the tyrosine kinase receptor B (TrkB), and an agonist of the TrkB receptor under the control of a single promoter. The promoter of the construct may be used to ensure that the agonist and the receptor are only expressed in appropriate nerve cells, and promote the survival of these cells.

Thus, according to a first aspect of the invention, there is provided a genetic construct comprising a promoter operably linked to a first coding sequence, which encodes the tyrosine kinase receptor B (TrkB), and a second coding sequence, which encodes an agonist of the TrkB receptor, for use in the treatment, prevention or amelioration of a neurodegenerative disorder or stroke.

The inventors have demonstrated in the Examples that it is possible to combine the genes which code for both the TrkB receptor and its agonist in a single genetic construct. This was especially challenging given their large sizes, and it could not have been predicted that it would have been possible to co-express them in physiologically useful concentrations. Advantageously, with the construct of the invention, there is no need to inject a recombinant protein, as described in the prior art [56]. Furthermore, in the prior art, it is still necessary to perform regular injections of protein, whereas the construct of the invention only requires a single gene therapy administration.

Preferably, in use, the TrkB receptor is activated by the agonist to thereby promote survival of nerve cells. The genetic construct of the invention is preferably used for the treatment, prevention or amelioration of a neurodegenerative disorder selected from a group consisting of: Alexander's disease, Alper's disease, Alzheimer's Disease, amyotrophic lateral sclerosis (ALS), ataxia telangiectasia, neuronal ceroid lipofuscinoses, Batten disease, bovine spongiform encephalopathy (BSE), Canavan disease, cerebral palsy, Cockayne syndrome, corticobasal degeneration, Creutzfeldt-Jakob disease, frontotemporal lobar degeneration, Gaucher's disease, Huntington's disease, HIV-associated dementia, Kennedy's disease, Krabbe's disease, Lewy body dementia, lysosomal storage disorders, neuroborreliosis, Machado-Joseph disease, motor neurone disease, multiple system atrophy, multiple sclerosis, multiple sulfatase deficiency, mucolipidoses, narcolepsy, Niemann-Pick type C, Niemann Pick disease, Parkinson's Disease, Pelizaeus-Merzbacher Disease, Pick's disease, Pompe disease, primary lateral sclerosis, prion diseases, progressive supranuclear palsy, Refsum's disease, Sandhoff disease, Schilder's disease, subacute combined degeneration of spinal cord secondary to pernicious anaemia, Spielmeyer-Vogt-Sjogren-Batten disease, spinocerebellar ataxia, spinal muscular atrophy, Steele-Richardson-Olszewski disease, Tabes dorsalis, and Tay-Sachs disease.

In a preferred embodiment, the genetic construct is used for the treatment, prevention or amelioration of Alzheimer's disease.

In a preferred embodiment, the genetic construct is for the treatment, prevention or amelioration of Huntington's disease.

In a preferred embodiment, the genetic construct is for the treatment, prevention or amelioration of Parkinson's disease.

In a preferred embodiment, the genetic construct is for the treatment, prevention or amelioration of motor neurone disease.

In a preferred embodiment, the genetic construct is for the treatment, prevention or amelioration of stroke.

The gene therapy construct may have several beneficial therapeutic effects for treating neurodegenerative disorders, such as Alzheimer's disease, or stroke. Benefits include therapeutically supplementing the depleted brain mBDNF concentrations, or supplementing with other trophic factors from the neurotrophin family. Other benefits include restoring TrkB receptor density levels in normal brain tissue. The potential to include an agonist in the genetic construct that has an absence of coding for the pro-sequence, for instance the absence of coding for proBDNF, also has the capability of restoring the balance in favour of mBDNF/TrkB type signalling and away from pro-BDNF/p75NTR type effects. Furthermore, as the gene therapy may be used to produce a mature form of the agonist, such as mBDNF, without generating pro-domain neurotrophin there will be a significantly lower risk of exacerbating the Aβ neurotoxicity, which could occur if the construct produced and released a pro-form of the agonist, such as proBDNF. Preferably, the construct of the invention is configured to reduce Tau phosphorylation in neurones (which is one of the pathophysiological features associated with Alzheimer brains).

Advantageously, the construct of the invention may therefore be used to target nerve cells in order to maintain or enhance TrkB-signalling in these cells. Thus, the construct may be used to maximise protection against pathophysiological stressors, and to promote nerve regeneration and/or survival. Furthermore, the construct may be used to provide long-term treatment of neurodegenerative disorders or strokes due to the expression of the TrkB receptor and an agonist of the receptor under the control of one or more promoter. Consequently, the construct has overcome the need to use multiple alternative treatments, which, even in combination, provide a transient therapeutic effect. Moreover, the construct of the invention is advantageous because it may be used to significantly enhance nerve cell sensitivity to TrkB receptor agonists due to a localised increase in both the TrkB receptor and the agonist of the receptor.

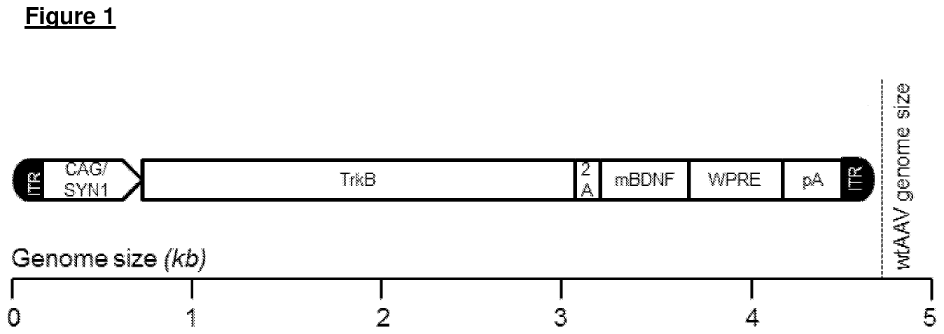

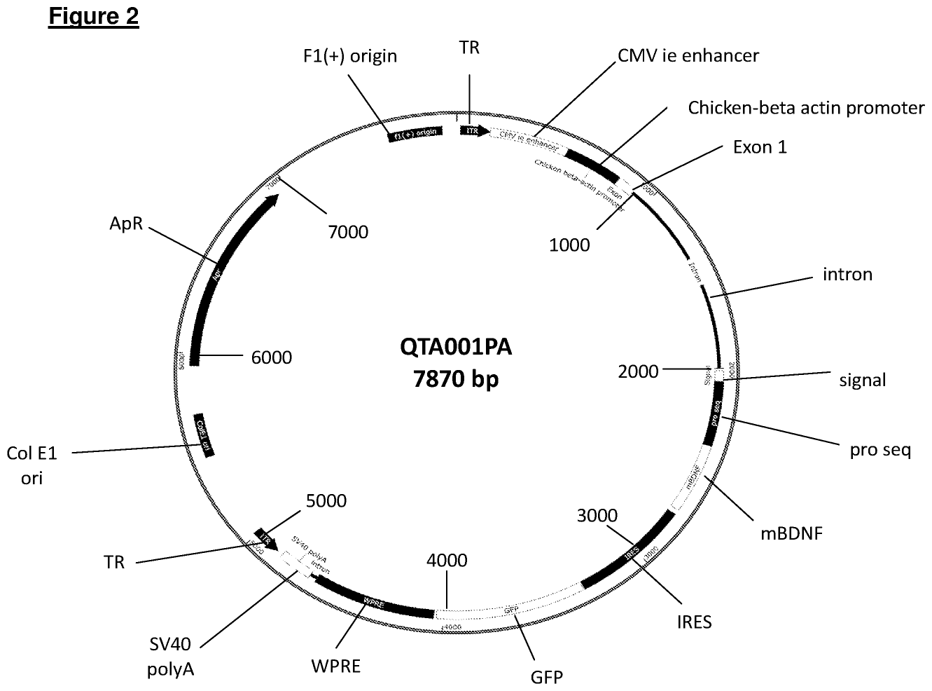

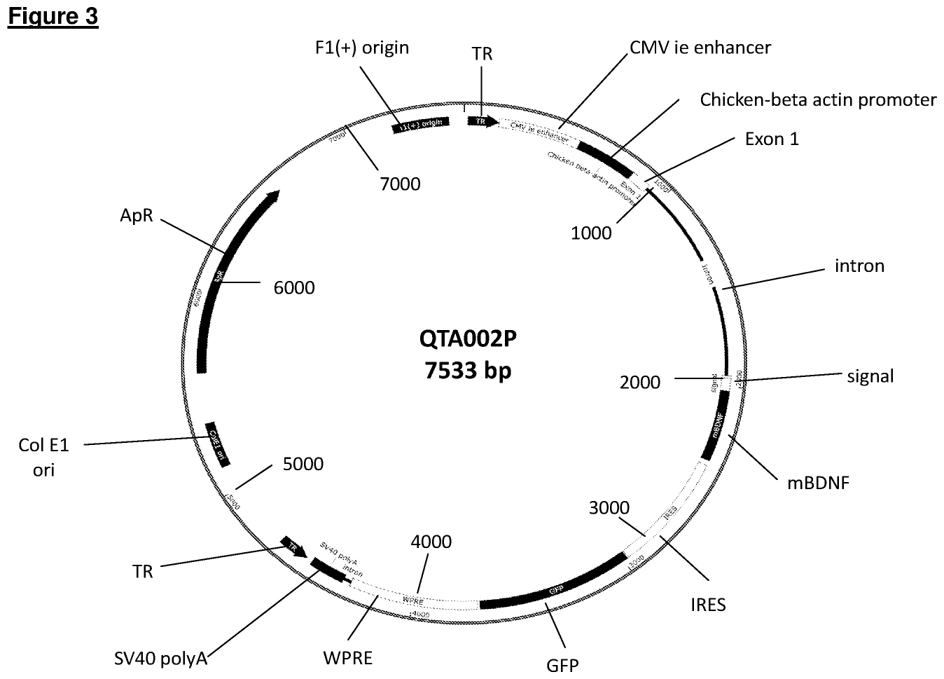

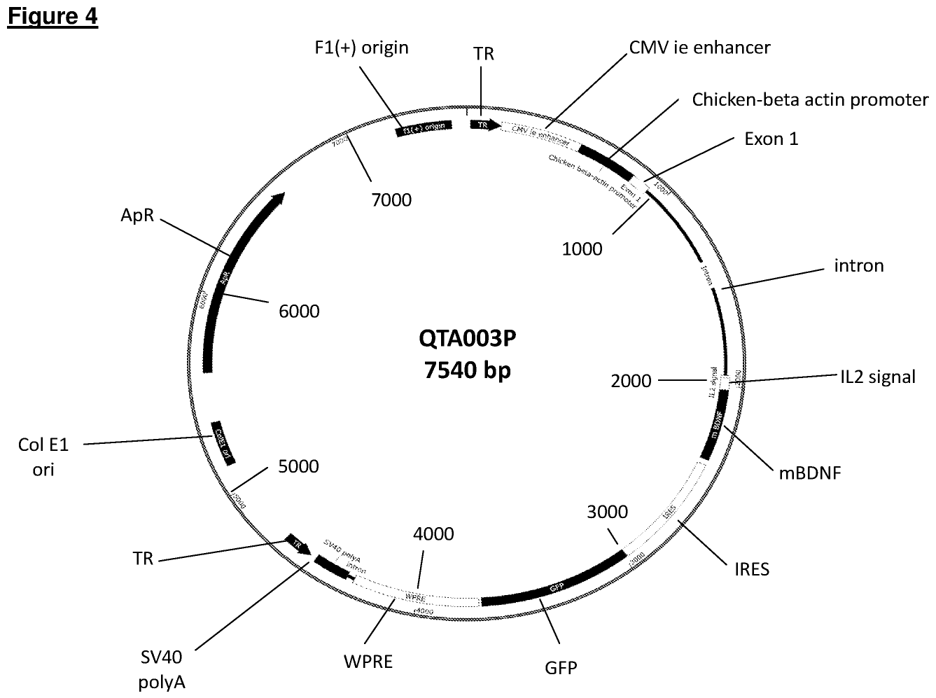

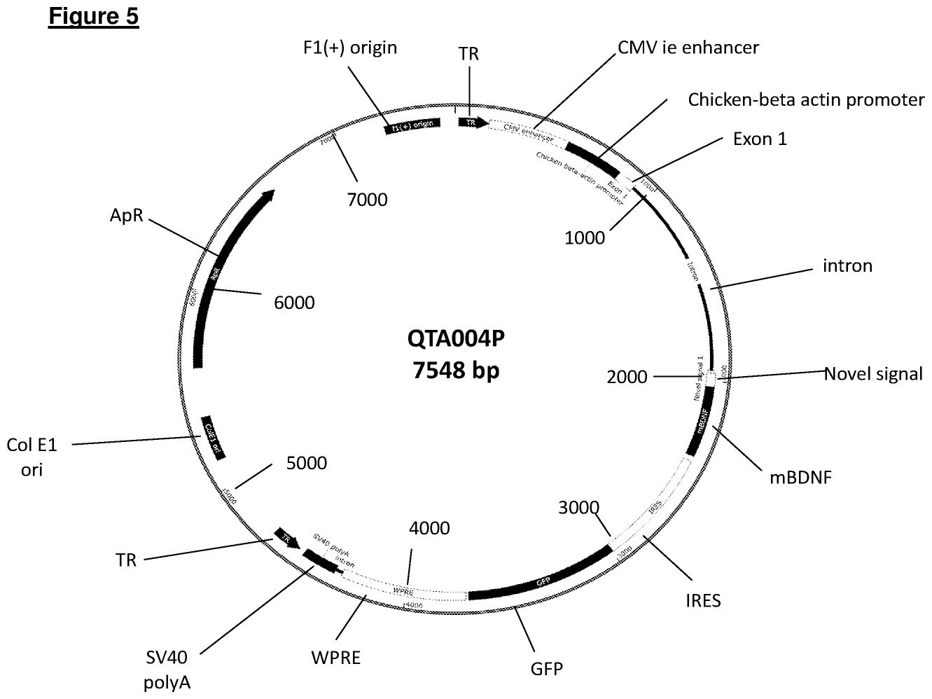

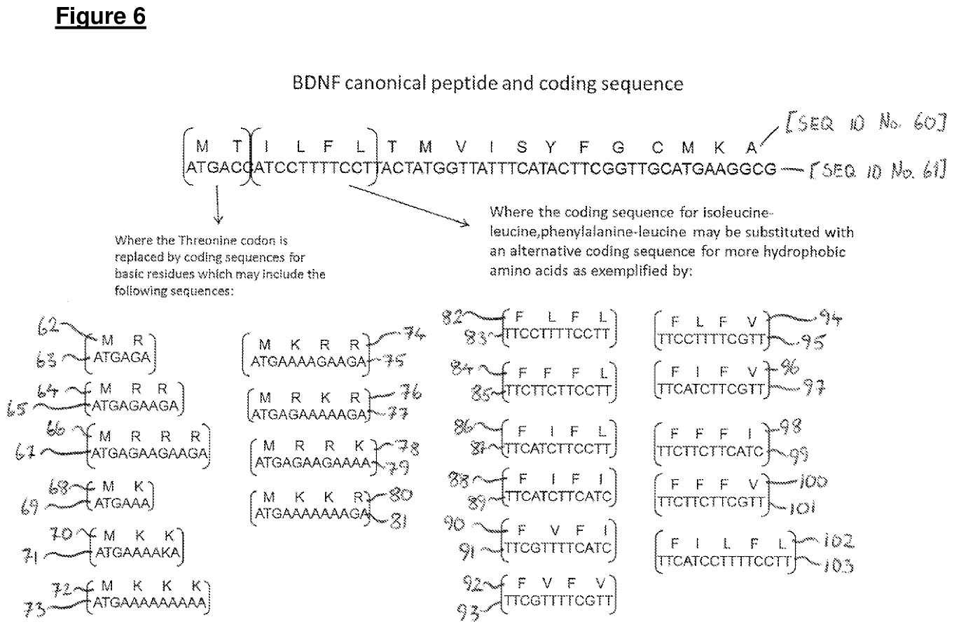

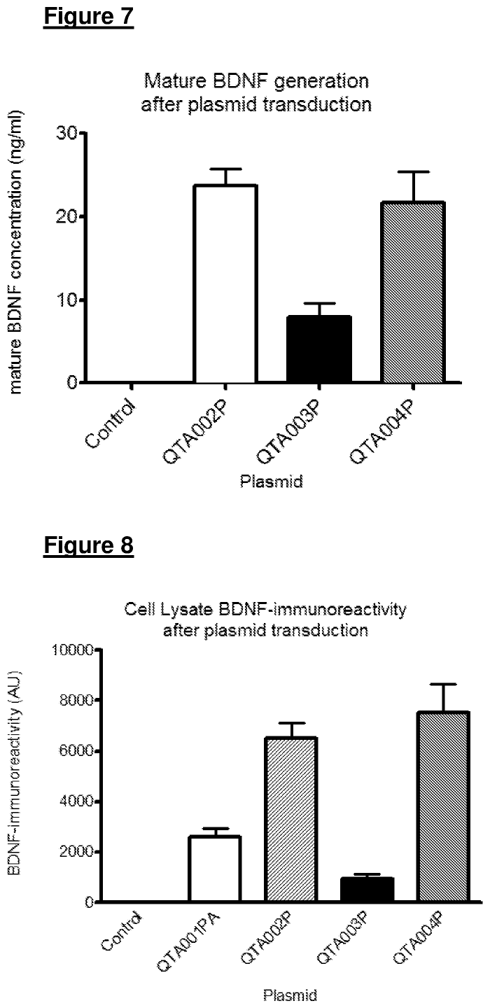

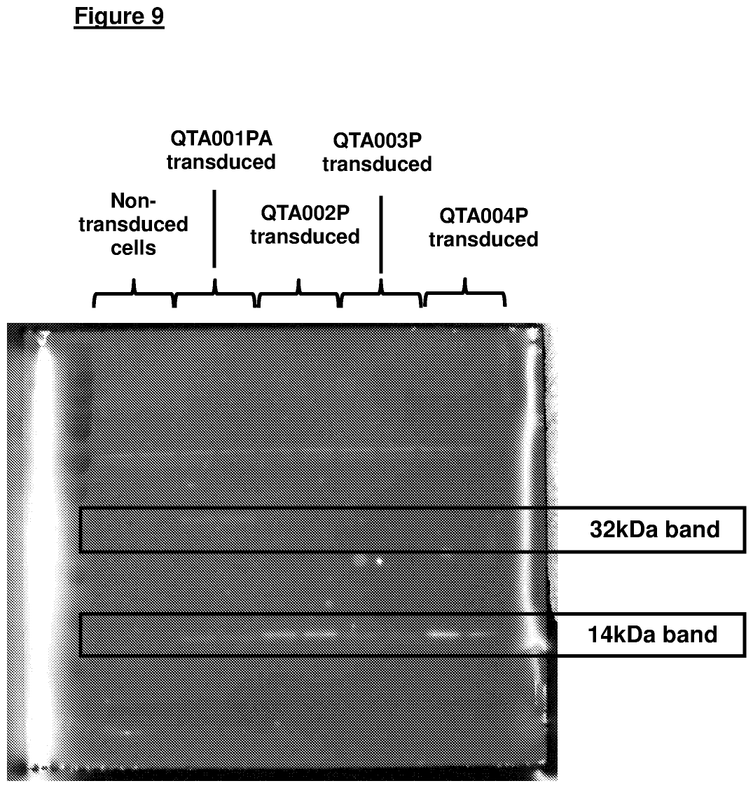

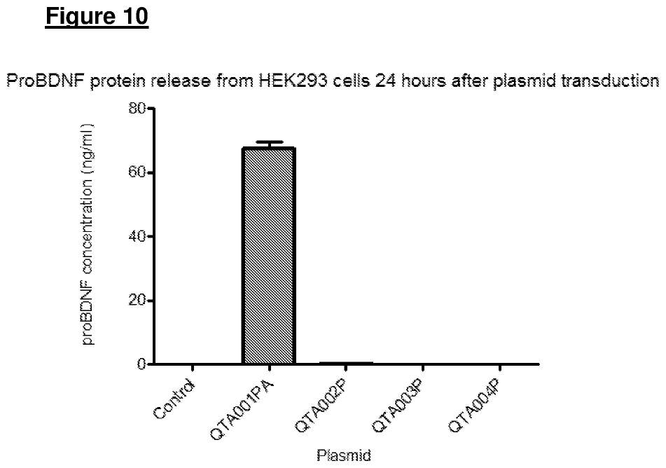

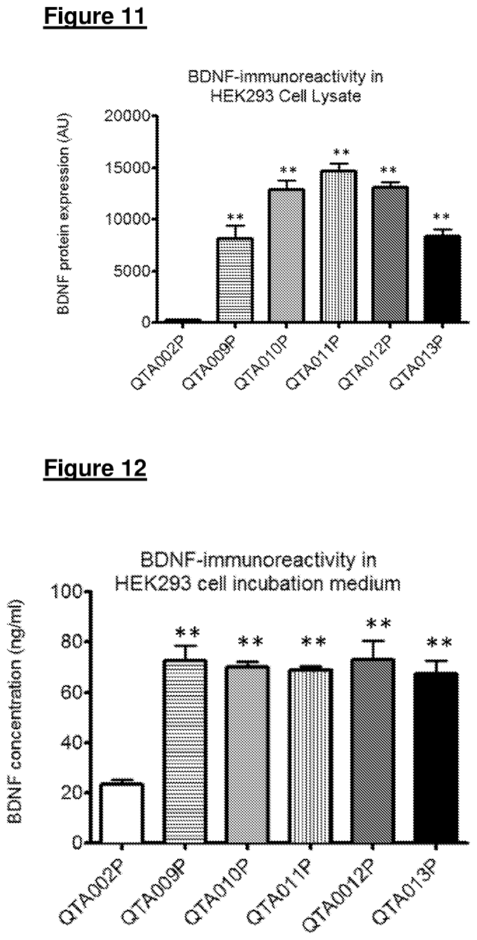

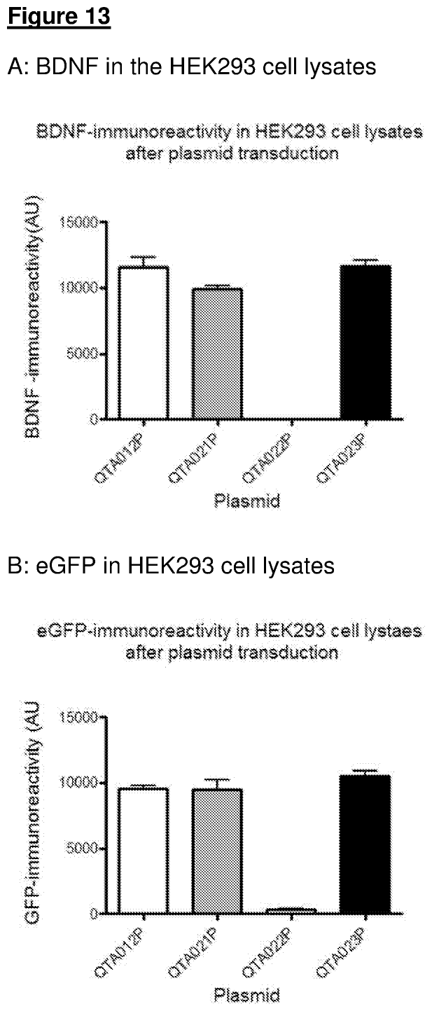

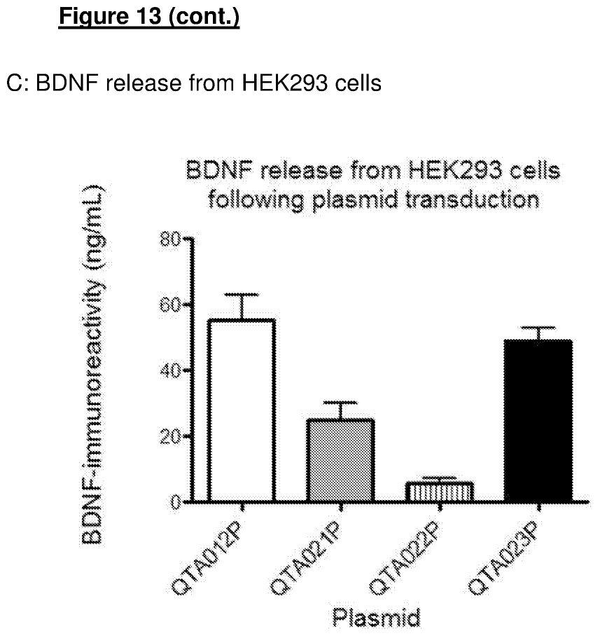

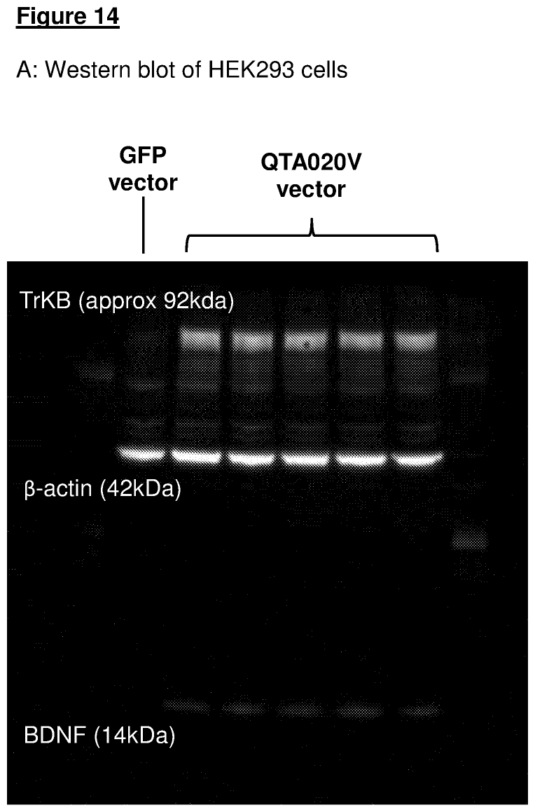

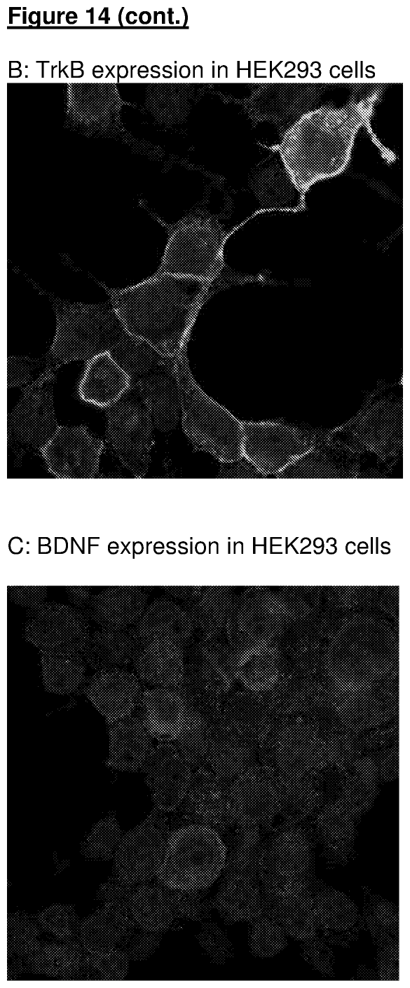

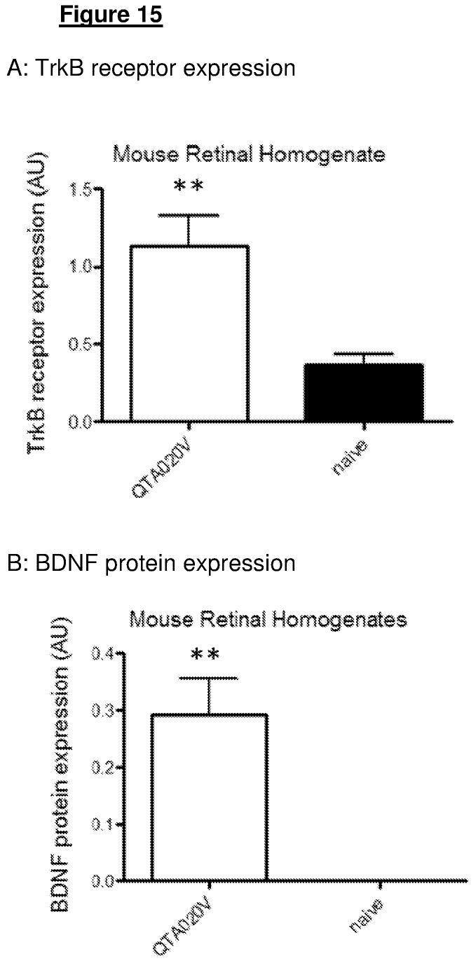

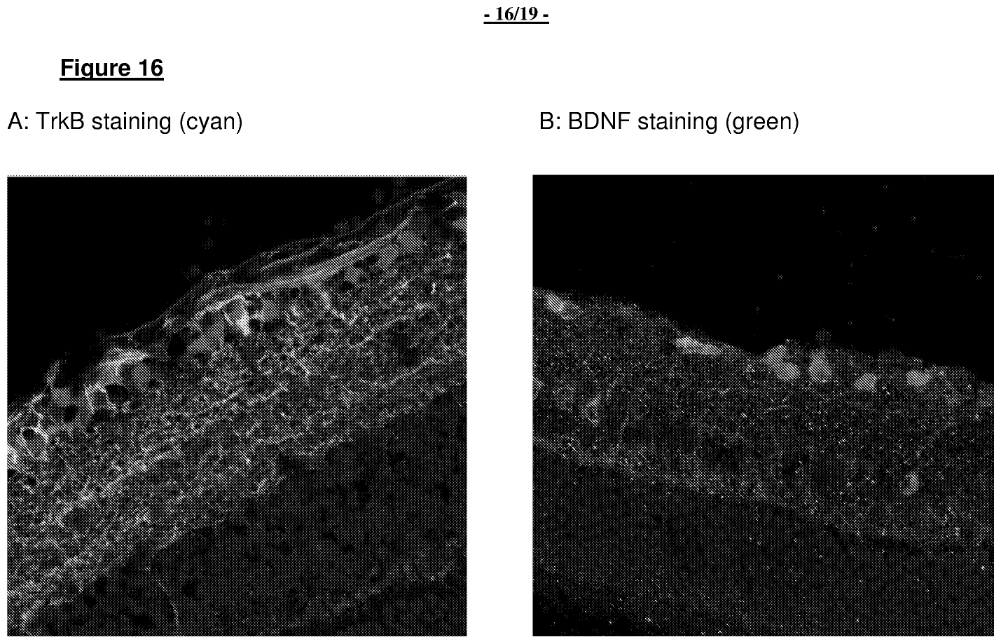

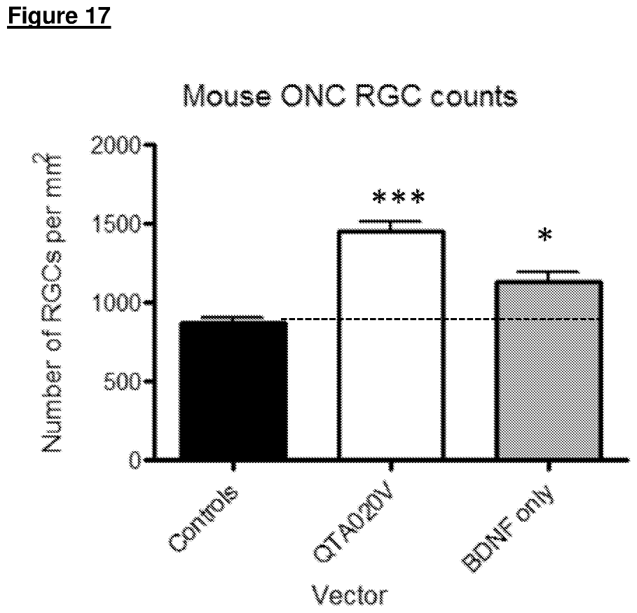

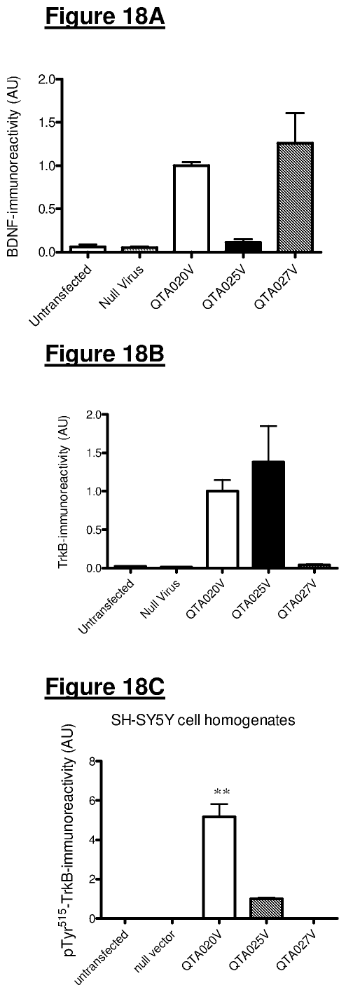

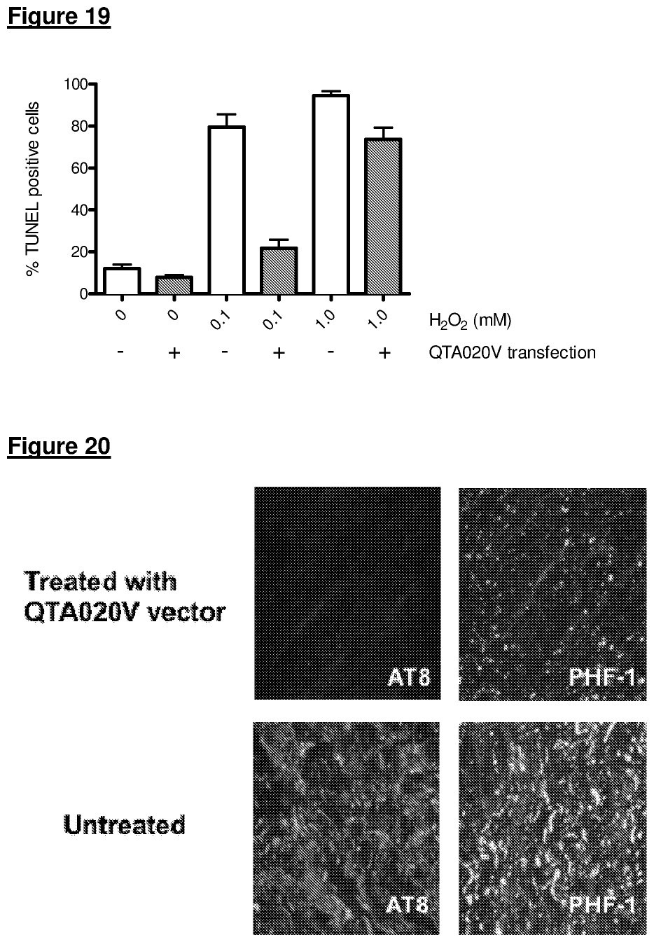

Preferably, the genetic construct of the invention comprises an expression cassette, one embodiment of which is shown in FIG. 1. As can be seen in FIG. 1, the construct comprises the promoter, the first nucleotide sequence encoding the TrkB receptor, and the second nucleotide sequence encoding mature brain derived neurotrophic (mBDNF), which acts as a preferred agonist of the TrkB receptor. It will be appreciated, however, that other agonist may be used, as discussed herein. Also as shown in FIG. 1, the expression cassette also includes a 2A spacer sequence, a sequence encoding Hepatitis Virus Post-transciptional Regulatory Element (WHPE), a sequence encoding a polyA tail, and left and right hand Inverted Terminal Repeat sequences (ITRs).

Hence, preferably the genetic construct comprises a spacer sequence disposed between the first and second coding sequences, which spacer sequence encodes a peptide spacer that is configured to be digested or cut to thereby produce the TrkB receptor and the agonist as separate molecules. In the embodiment illustrated in FIG. 1, the coding sequence for the TrkB receptor is disposed 5′ of the coding sequence for the receptor agonist (BDNF) with the spacer sequence therebetween. However, in another embodiment, the coding sequence for the receptor agonist may be disposed 5′ of the coding sequence for the receptor with the spacer sequence therebetween.

Preferably, the genetic construct comprises a nucleotide sequence encoding Woodchuck Hepatitis Virus Post-transcriptional Regulatory Element (WHPE), which enhances the expression of the two transgenes, i.e. the TrkB receptor and its agonist, which is preferably BDNF. Preferably, the WHPE coding sequence is disposed 3′ of the transgene coding sequence.

One embodiment of the Woodchuck Hepatitis Virus Post-transcriptional Regulatory Element (WHPE) is 592 bp long, including gamma-alpha-beta elements, and is referred to herein as SEQ ID No: 57, as follows:

[SEQ ID NO. 57]
AATCAACCTCTGGATTACAAAATTTGTGAAAGATTGACTGGTATTCTTAA
CTATGTTGCTCCTTTTACGCTATGTGGATACGCTGCTTTAATGCCTTTGT
ATCATGCTATTGCTTCCCGTATGGCTTTCATTTTCTCCTCCTTGTATAAA
TCCTGGTTGCTGTCTCTTTATGAGGAGTTGTGGCCCGTTGTCAGGCAACG
TGGCGTGGTGTGCACTGTGTTTGCTGACGCAACCCCCACTGGTTGGGGCA
TTGCCACCACCTGTCAGCTCCTTTCCGGGACTTTCGCTTTCCCCCTCCCT
ATTGCCACGGCGGAACTCATCGCCGCCTGCCTTGCCCGCTGCTGGACAGG
GGCTCGGCTGTTGGGCACTGACAATTCCGTGGTGTTGTCGGGGAAGCTGA
CGTCCTTTCCATGGCTGCTCGCCTGTGTTGCCACCTGGATTCTGCGCGGG
ACGTCCTTCTGCTACGTCCCTTCGGCCCTCAATCCAGCGGACCTTCCTTC
CCGCGGCCTGCTGCCGGCTCTGCGGCCTCTTCCGCGTCTTCGCCTTCGCC
CTCAGACGAGTCGGATCTCCCTTTGGGCCGCCTCCCCGCCTG

Preferably, the WHPE comprises a nucleic acid sequence substantially as set out in SEQ ID No: 57, or a fragment or variant thereof.

However, in a preferred embodiment, a truncated WHPE is used, which is 247 bp long due to deletion of the beta element, and which is referred to herein as SEQ ID No: 58, as follows:

[SEQ ID NO. 58]
AATCAACCTCTGGATTACAAAATTTGTGAAAGATTGACTGGTATTCTTAA
CTATGTTGCTCCTTTTACGCTATGTGGATACGCTGCTTTAATGCCTTTGT
ATCATGCTATTGCTTCCCGTATGGCTTTCATTTTCTCCTCCTTGTATAAA
TCCTGGTTAGTTCTTGCCACGGCGGAACTCATCGCCGCCTGCCTTGCCCG
CTGCTGGACAGGGGCTCGGCTGTTGGGCACTGACAATTCCGTGGTGT

Advantageously, the truncated WHPE sequence used in the construct saved about 300 bp in total without negatively impacting on transgene expression. Preferably, the WHPE comprises a nucleic acid sequence substantially as set out in SEQ ID No: 58, or a fragment or variant thereof.

Preferably, the genetic construct comprises a nucleotide sequence encoding a polyA tail. Preferably, the polyA tail coding sequence is disposed 3′ of the transgene coding sequence, and preferably 3′ of the WHPE coding sequence.

Preferably, the polyA tail comprises the simian virus 40 poly-A 224 bp sequence. One embodiment of the polyA tail is referred to herein as SEQ ID No: 59, as follows:

[SEQ ID NO. 59]
AGCAGACATGATAAGATACATTGATGAGTTTGGACAAACCACAACTAGAA
TGCAGTGAAAAAAATGCTTTATTTGTGAAATTTGTGATGCTATTGCTTTA
TTTGTAACCATTATAAGCTGCAATAAACAAGTTAACAACAACAATTGCAT
TCATTTTATGTTTCAGGTTCAGGGGGAGGTGTGGGAGGTTTTTTAAAGCA
AGTAAAACCTCTACAAATGTGGTA

Preferably, the polyA tail comprises a nucleic acid sequence substantially as set out in SEQ ID No: 59, or a fragment or variant thereof.

Preferably, the genetic construct comprises left and/or right Inverted Terminal Repeat sequences (ITRs). Preferably, each ITR is disposed at the 5′ and/or 3′ end of the construct.

The promoter in the genetic construct of the first aspect may be any nucleotide sequence that is capable of inducing RNA polymerase to bind to and transcribe the first and second coding sequences. In one embodiment, the promoter in the genetic construct of the first aspect may be the cytomelalovirus (CMV) constitutive promoter. This is believes to be non-selective for both neuronal and glial cells.

In one preferred embodiment, the promoter is the human synapsin I (SYN I) promoter, which has been shown to work in human brain. One embodiment of the 469 nucleotide sequence encoding the human synapsin I (SYN I) promoter is referred to herein as SEQ ID NO.1, as follows:

[SEQ ID NO. 1]
CTGCAGAGGGCCCTGCGTATGAGTGCAAGTGGGTTTTAGGACCAGGATGA
GGCGGGGTGGGGGTGCCTACCTGACGACCGACCCCGACCCACTGGACAAG
CACCCAACCCCCATTCCCCAAATTGCGCATCCCCTATCAGAGAGGGGGAG
GGGAAACAGGATGCGGCGAGGCGCGTGCGCACTGCCAGCTTCAGCACCGC
GGACAGTGCCTTCGCCCCCGCCTGGCGGCGCGCGCCACCGCCGCCTCAGC
ACTGAAGGCGCGCTGACGTCACTCGCCGGTCCCCCGCAAACTCCCCTTCC
CGGCCACCTTGGTCGCGTCCGCGCCGCCGCCGGCCCAGCCGGACCGCACC
ACGCGAGGCGCGAGATAGGGGGGCACGGGCGCGACCATCTGCGCTGCGGC
GCCGGCGACTCAGCGCTGCCTCAGTCTGCGGTGGGCAGCGGAGGAGTCGT
GTCGTGCCTGAGAGCGCAG

Preferably, therefore, the promoter may comprise a nucleotide acid sequence substantially as set out in SEQ ID No: 1, or a fragment or variant thereof.

In another preferred embodiment, the promoter is the CAG promoter, which has also been shown to work in human brain. The CAG promoter preferably comprises the cytomegalovirus early enhancer element, the first exon and the first intron of chicken beta-actin gene and the splice acceptor of the rabbit beta-globin gene. One embodiment of the 1733 nucleotide sequence encoding the CAG promoter is referred to herein as SEQ ID NO.2, as follows:

[SEQ ID NO. 2]
CTCGACATTGATTATTGACTAGTTATTAATAGTAATCAATTACGGGGTCA
TTAGTTCATAGCCCATATATGGAGTTCCGCGTTACATAACTTACGGTAAA
TGGCCCGCCTGGCTGACCGCCCAACGACCCCCGCCCATTGACGTCAATAA
TGACGTATGTTCCCATAGTAACGCCAATAGGGACTTTCCATTGACGTCAA
TGGGTGGAGTATTTACGGTAAACTGCCCACTTGGCAGTACATCAAGTGTA
TCATATGCCAAGTACGCCCCCTATTGACGTCAATGACGGTAAATGGCCCG
CCTGGCATTATGCCCAGTACATGACCTTATGGGACTTTCCTACTTGGCAG
TACATCTACGTATTAGTCATCGCTATTACCATGGTCGAGGTGAGCCCCAC
GTTCTGCTTCACTCTCCCCATCTCCCCCCCCTCCCCACCCCCAATTTTGT
ATTTATTTATTTTTTAATTATTTTGTGCAGCGATGGGGGCGGGGGGGGGG
GGGGGGCGCGCGCCAGGCGGGGCGGGGCGGGGCGAGGGGCGGGGCGGGGC
GAGGCGGAGAGGTGCGGCGGCAGCCAATCAGAGCGGCGCGCTCCGAAAGT
TTCCTTTTATGGCGAGGCGGCGGCGGCGGCGGCCCTATAAAAAGCGAAGC
GCGCGGCGGGCGGGAGTCGCTGCGCGCTGCCTTCGCCCCGTGCCCCGCTC
CGCCGCCGCCTCGCGCCGCCCGCCCCGGCTCTGACTGACCGCGTTACTCC
CACAGGTGAGCGGGCGGGACGGCCCTTCTCCTCCGGGCTGTAATTAGCGC
TTGGTTTAATGACGGCTTGTTTCTTTTCTGTGGCTGCGTGAAAGCCTTGA
GGGGCTCCGGGAGGGCCCTTTGTGCGGGGGGAGCGGCTCGGGGGGTGCGT
GCGTGTGTGTGTGCGTGGGGAGCGCCGCGTGCGGCTCCGCGCTGCCCGGC
GGCTGTGAGCGCTGCGGGCGCGGCGCGGGGCTTTGTGCGCTCCGCAGTGT
GCGCGAGGGGAGCGCGGCCGGGGGCGGTGCCCCGCGGTGCGGGGGGGGCT
GCGAGGGGAACAAAGGCTGCGTGCGGGGTGTGTGCGTGGGGGGGTGAGCA
GGGGGTGTGGGCGCGTCGGTCGGGCTGCAACCCCCCCTGCACCCCCCTCC
CCGAGTTGCTGAGCACGGCCCGGCTTCGGGTGCGGGGCTCCGTACGGGGC
GTGGCGCGGGGCTCGCCGTGCCGGGCGGGGGGTGGCGGCAGGTGGGGGTG
CCGGGCGGGGCGGGGCCGCCTCGGGCCGGGGAGGGCTCGGGGGAGGGGCG
CGGCAGGCCCCCGGGCGCCGGCGGCTGTCGAGGCGCGGCGAGCCGCAGCC
ATTGCCTTTTATGGTAATCGTGCGAGAGGGCGCAGGGACTTCCTTTGTCC
CAAATCTGTGCGGAGCCGAAATCTGGGAGGCGCCGCCGCACCCCCTCTAG
CGGGCGCGGGGCGAAGCGGTGCGGCGCCGGCAGGAAGGAAATGGGCGGGG
AGGGCCTTCGTGCGTCGCCGCGCCGCCGTCCCCTTCTCCCTCTCCAGCCT
CGGGGCTGTCCGCGGGGGGACGGCTGCCTTCGGGGGGGACGGGGCAGGGC
GGGGTTCGGCTTCTGGCGTGTGACCGGCGGCTCTAGAGCCTCTGCTAACC
ATGTTCATGCCTTCTTCTTTTTCCTACAGCTCCTGGGCAACGTGCTGGTT
ATTGTGCTGTCTCATCATTTTGGCAAAGAATTG

In another preferred embodiment, the promoter is a truncated form of the CAG promoter, such as a 664 nucleotide form of the promoter referred to herein as SEQ ID NO.3, as follows:

[SEQ ID No: 3]
CTAGATCTGAATTCGGTACCCTAGTTATTAATAGTAATCAATTACGGGGT
CATTAGTTCATAGCCCATATATGGAGTTCCGCGTTACATAACTTACGGTA
AATGGCCCGCCTGGCTGACCGCCCAACGACCCCCGCCCATTGACGTCAAT
AATGACGTATGTTCCCATAGTAACGCCAATAGGGACTTTCCATTGACGTC
AATGGGTGGACTATTTACGGTAAACTGCCCACTTGGCAGTACATCAAGTG
TATCATATGCCAAGTACGCCCCCTATTGACGTCAATGACGGTAAATGGCC
CGCCTGGCATTATGCCCAGTACATGACCTTATGGGACTTTCCTACTTGGC
AGTACATCTACGTATTAGTCATCGCTATTACCATGGTCGAGGTGAGCCCC
ACGTTCTGCTTCACTCTCCCCATCTCCCCCCCCTCCCCACCCCCAATTTT
GTATTTATTTATTTTTTAATTATTTTGTGCAGCGATGGGGGCGGGGGGGG
GGGGGGGGCGCGCGCCAGGCGGGGCGGGGCGGGGCGAGGGGCGGGGCGGG
GCGAGGCGGAGAGGTGCGGCGGCAGCCAATCAGAGCGGCGCGCTCCGAAA
GTTTCCTTTTATGGCGAGGCGGCGGCGGCGGCGGCCCTATAAAAAGCGAA
GCGCGCGGCGGGCG

In yet a further preferred embodiment, the promoter is a truncated form of the CAG promoter, such as a 584 nucleotide form of the promoter referred to herein as SEQ ID NO. 48, as follows:

[SEQ ID No: 48]
GCGTTACATAACTTACGGTAAATGGCCCGCCTGGCTGACCGCCCAACGAC
CCCCGCCCATTGACGTCAATAATGACGTATGTTCCCATAGTAACGCCAAT
AGGGACTTTCCATTGACGTCAATGGGTGGACTATTTACGGTAAACTGCCC
ACTTGGCAGTACATCAAGTGTATCATATGCCAAGTACGCCCCCTATTGAC
GTCAATGACGGTAAATGGCCCGCCTGGCATTATGCCCAGTACATGACCTT
ATGGGACTTTCCTACTTGGCAGTACATCTACGTATTAGTCATCGCTATTA
CCATGGTCGAGGTGAGCCCCACGTTCTGCTTCACTCTCCCCATCTCCCCC
CCCTCCCCACCCCCAATTTTGTATTTATTTATTTTTTAATTATTTTGTGC
AGCGATGGGGGCGGGGGGGGGGGGGGGGCGCGCGCCAGGCGGGGCGGGGC
GGGGCGAGGGGCGGGGCGGGGCGAGGCGGAGAGGTGCGGCGGCAGCCAAT
CAGAGCGGCGCGCTCCGAAAGTTTCCTTTTATGGCGAGGCGGCGGCGGCG
GCGGCCCTATAAAAAGCGAAGCGCGCGGCGGGCG

Therefore, preferably the promoter comprises a nucleotide acid sequence substantially as set out in SEQ ID No: 2, 3 or 48, or a fragment or variant thereof.

Many bicistronic gene constructs presented in the scientific literature have either (i) incorporated dual promoters to separately drive expression of two genes, or (ii) use the internal ribosome entry site (IRES) of the encepahlomyocarditis virus (EMCV) to link two genes transcribed from a single promoter within recombinant viral vectors [45-46]. However, the efficiency of IRES-dependent translation may vary in different cells and tissues and IRES-dependent second gene expression can be significantly lower than cap-dependent first gene expression in bicistronic vectors [47]. Moreover, the size limitation of rAAV vectors (generally <5 kb) will prevent the incorporation of large gene constructs, such as the TrkB receptor together with BDNF using dual promoters or IRES linkers.

Accordingly, in a preferred embodiment, the genetic construct comprises a spacer sequence disposed between the first and second coding sequences, which spacer sequence encodes a peptide spacer that is configured to be digested to thereby produce the TrkB receptor and agonist as separate molecules. Preferably, the spacer sequence comprises and encodes a viral peptide spacer sequence, more preferably a viral 2A peptide spacer sequence [47]. Preferably, the 2A peptide sequence connects the first coding sequence to the second coding sequence. This enables the construct to overcome the size restrictions that occur with expression in various vectors and enables expression of all of the peptides encoded by the construct of the first aspect to occur under control of a single promoter, as a single protein.

Thus, following the translation of the single protein containing the sequences of TrkB, the 2A peptide, and the agonist (preferably BDNF), cleavage occurs in the viral 2A peptide sequence at the terminal glycine-proline link, thereby liberating two proteins, i.e. TrkB and agonist (e.g. mBDNF). The genetic construct is designed such that the remaining short N-terminal amino acid sequence of the viral 2A peptide remain attached to the intracellular portion of the TrkB receptor, thereby removing immunogenicity risks and not interfering with the intracellular signalling capability of the mature receptor. The residual proline amino acid from the C-terminal viral 2A sequence remains attached to the N-terminal agonist signal peptide and is ultimately removed from the agonist protein following cleavage of the signal sequence from the mature protein.

The inventors have generated two embodiments of the spacer sequence. One important section of the peptide spacer sequence, which is common to both embodiments described herein, is the C-terminus. Accordingly, preferably the peptide spacer sequence comprises an amino acid sequence referred to herein as SEQ ID NO. 4, or a fragment or variant thereof, as follows:

| | |
|---|---|
| QAGDVEENPGP | [SEQ ID No: 4] |

Preferably, the digestion or cut site of the peptide spacer sequence is disposed between the terminal glycine and end proline in SEQ ID No:4.

In a first preferred embodiment, the spacer sequence comprises a nucleotide sequence referred to herein as SEQ ID NO.5, or a fragment or variant thereof, as follows:

[SEQ ID No: 5]
GGAAGCGGAGCTACTAACTTCAGCCTGCTGAAGGCTGGAGACGTGGAGGA
GAACCCTGGACCT

In this first embodiment, the peptide spacer sequence comprises an amino acid sequence referred to herein as SEQ ID NO. 6, or a fragment or variant thereof, as follows:

| | |
|---|---|
| GSGATNFSLLQAGDVEENPGP | [SEQ ID No: 6] |

In a second preferred embodiment, the spacer sequence comprises a nucleotide sequence referred to herein as SEQ ID NO. 7, or a fragment or variant thereof, as follows:

[SEQ ID No: 7]
AGCGGAGCTACTAACTTCAGCCTGCTGAAGCAGGCTGGAGACGTGGAGGA
GAACCCTGGACCT

In this second embodiment, the peptide spacer sequence comprises an amino acid sequence referred to herein as SEQ ID NO. 8, or a fragment or variant thereof, as follows:

| | |
|---|---|
| SGATNFSLLKQAGDVEENPGP | [SEQ ID No: 8] |

The inventors have carefully considered the sequences of the TrkB receptor, and have produced several preferred embodiments of the receptor that is encoded by the first coding sequence in the genetic construct of the first aspect.

In one preferred embodiment, the first coding sequence comprises a nucleotide sequence encoding the human canonical isoform of TrkB. Preferably, the canonical isoform of TrkB comprises an amino acid sequence (822 residues) referred to herein as SEQ ID NO. 9, or a fragment or variant thereof, as set out below:

[SEQ ID No: 9]
MSSWIRWHGPAMARLWGFCWLVVGFWRAAFACPTSCKCSASRIWCSDPSP
GIVAFPRLEPNSVDPENITEIFIANQKRLEIINEDDVEAYVGLRNLTIVD
SGLKFVAHKAFLKNSNLQHINFTRNKLTSLSRKHFRHLDLSELILVGNPF
TCSCDIMWIKTLQEAKSSPDTQDLYCLNESSKNIPLANLQIPNCGLPSAN
LAAPNLTVEEGKSITLSCSVAGDPVPNMYWDVGNLVSKHMNETSHTQGSL
RITNISSDDSGKQISCVAENLVGEDQDSVNLTVHFAPTITFLESPTSDHH
WCIPFTVKGNPKPALQWFYNGAILNESKYICTKIHVTNHTEYHGCLQLDN
PTHMNNGDYTLIAKNEYGKDEKQISAHFMGWPGIDDGANPNYPDVIYEDY
GTAANDIGDTTNRSNEIPSTDVTDKTGREHLSVYAVVVIASVVGFCLLVM
LFLLKLARHSKFGMKGPASVISNDDDSASPLHHISNGSNTPSSSEGGPDA
VIIGMTKIPVIENPQYFGITNSQLKPDTFVQHIKRHNIVLKRELGEGAFG
KVFLAECYNLCPEQDKILVAVKTLKDASDNARKDFHREAELLTNLQHEHI
VKFYGVCVEGDPLIMVFEYMKHGDLNKFLRAHGPDAVLMAEGNPPTELTQ
SQMLHIAQQIAAGMVYLASQHFVHRDLATRNCLVGENLLVKIGDFGMSRD
VYSTDYYRVGGHTMLPIRWMPPESIMYRKFTTESDVWSLGVVLWEIFTYG
KQPWYQLSNNEVIECITQGRVLQRPRTCPQEVYELMLGCWQREPHMRKNI
KGIHTLLQNLAKASPVYLDILG

Preferably, in this embodiment, the first coding sequence comprises a nucleotide sequence referred to herein as SEQ ID NO. 10, or a fragment or variant thereof, as set out below:

[SEQ ID No: 10]
ATGTCGTCCTGGATAAGGTGGCATGGACCCGCCATGGCGCGGCTCTGGGG
CTTCTGCTGGCTGGTTGTGGGCTTCTGGAGGGCCGCTTTCGCCTGTCCCA
CGTCCTGCAAATGCAGTGCCTCTCGGATCTGGTGCAGCGACCCTTCTCCT
GGCATCGTGGCATTTCCGAGATTGGAGCCTAACAGTGTAGATCCTGAGAA
CATCACCGAAATTTTCATCGCAAACCAGAAAAGGTTAGAAATCATCAACG
AAGATGATGTTGAAGCTTATGTGGGACTGAGAAATCTGACAATTGTGGAT
TCTGGATTAAAATTTGTGGCTCATAAAGCATTTCTGAAAAACAGCAACCT
GCAGCACATCAATTTTACCCGAAACAAACTGACGAGTTTGTCTAGGAAAC
ATTTCCGTCACCTTGACTTGTCTGAACTGATCCTGGTGGGCAATCCATTT
ACATGCTCCTGTGACATTATGTGGATCAAGACTCTCCAAGAGGCTAAATC
CAGTCCAGACACTCAGGATTTGTACTGCCTGAATGAAAGCAGCAAGAATA
TTCCCCTGGCAAACCTGCAGATACCCAATTGTGGTTTGCCATCTGCAAAT
CTGGCCGCACCTAACCTCACTGTGGAGGAAGGAAAGTCTATCACATTATC
CTGTAGTGTGGCAGGTGATCCGGTTCCTAATATGTATTGGGATGTTGGTA
ACCTGGTTTCCAAACATATGAATGAAACAAGCCACACACAGGGCTCCTTA
AGGATAACTAACATTTCATCCGATGACAGTGGGAAGCAGATCTCTTGTGT
GGCGGAAAATCTTGTAGGAGAAGATCAAGATTCTGTCAACCTCACTGTGC
ATTTTGCACCAACTATCACATTTCTCGAATCTCCAACCTCAGACCACCAC
TGGTGCATTCCATTCACTGTGAAAGGCAACCCCAAACCAGCGCTTCAGTG
GTTCTATAACGGGGCAATATTGAATGAGTCCAAATACATCTGTACTAAAA
TACATGTTACCAATCACACGGAGTACCACGGCTGCCTCCAGCTGGATAAT
CCCACTCACATGAACAATGGGGACTACACTCTAATAGCCAAGAATGAGTA
TGGGAAGGATGAGAAACAGATTTCTGCTCACTTCATGGGCTGGCCTGGAA
TTGACGATGGTGCAAACCCAAATTATCCTGATGTAATTTATGAAGATTAT
GGAACTGCAGCGAATGACATCGGGGACACCACGAACAGAAGTAATGAAAT
CCCTTCCACAGACGTCACTGATAAAACCGGTCGGGAACATCTCTCGGTCT
ATGCTGTGGTGGTGATTGCGTCTGTGGTGGGATTTTGCCTTTTGGTAATG
CTGTTTCTGCTTAAGTTGGCAAGACACTCCAAGTTTGGCATGAAAGGCCC
AGCCTCCGTTATCAGCAATGATGATGACTCTGCCAGCCCACTCCATCACA
TCTCCAATGGGAGTAACACTCCATCTTCTTCGGAAGGTGGCCCAGATGCT
GTCATTATTGGAATGACCAAGATCCCTGTCATTGAAAATCCCCAGTACTT
TGGCATCACCAACAGTCAGCTCAAGCCAGACACATTTGTTCAGCACATCA
AGCGACATAACATTGTTCTGAAAAGGGAGCTAGGCGAAGGAGCCTTTGGA
AAAGTGTTCCTAGCTGAATGCTATAACCTCTGTCCTGAGCAGGACAAGAT
CTTGGTGGCAGTGAAGACCCTGAAGGATGCCAGTGACAATGCACGCAAGG
ACTTCCACCGTGAGGCCGAGCTCCTGACCAACCTCCAGCATGAGCACATC
GTCAAGTTCTATGGCGTCTGCGTGGAGGGCGACCCCCTCATCATGGTCTT
TGAGTACATGAAGCATGGGGACCTCAACAAGTTCCTCAGGGCACACGGCC
CTGATGCCGTGCTGATGGCTGAGGGCAACCCGCCCACGGAACTGACGCAG
TCGCAGATGCTGCATATAGCCCAGCAGATCGCCGCGGGCATGGTCTACCT
GGCGTCCCAGCACTTCGTGCACCGCGATTTGGCCACCAGGAACTGCCTGG
TCGGGGAGAACTTGCTGGTGAAAATCGGGGACTTTGGGATGTCCCGGGAC
GTGTACAGCACTGACTACTACAGGGTCGGTGGCCACACAATGCTGCCCAT
TCGCTGGATGCCTCCAGAGAGCATCATGTACAGGAAATTCACGACGGAAA
GCGACGTCTGGAGCCTGGGGGTCGTGTTGTGGGAGATTTTCACCTATGGC
AAACAGCCCTGGTACCAGCTGTCAAACAATGAGGTGATAGAGTGTATCAC
TCAGGGCCGAGTCCTGCAGCGACCCCGCACGTGCCCCCAGGAGGTGTATG
AGCTGATGCTGGGGTGCTGGCAGCGAGAGCCCCACATGAGGAAGAACATC
AAGGGCATCCATACCCTCCTTCAGAACTTGGCCAAGGCATCTCCGGTCTA
CCTGGACATTCTAGGC

In another preferred embodiment, the first coding sequence comprises a nucleotide sequence which encodes isoform 4 of TrkB. Preferably, isoform 4 of TrkB comprises an amino acid sequence referred to herein as SEQ ID NO. 11, or a fragment or variant thereof, as set out below:

[SEQ ID No: 11]
MSSWIRWHGPAMARLWGFCWLVVGFWRAAFACPTSCKCSASRIWCSDPSP
GIVAFPRLEPNSVDPENITEIFIANQKRLEIINEDDVEAYVGLRNLTIVD
SGLKFVAHKAFLKNSNLQHINFTRNKLTSLSRKHFRHLDLSELILVGNPF
TCSCDIMWIKTLQEAKSSPDTQDLYCLNESSKNIPLANLQIPNCGLPSAN
LAAPNLTVEEGKSITLSCSVAGDPVPNMYWDVGNLVSKHMNETSHTQGSL
RITNISSDDSGKQISCVAENLVGEDQDSVNLTVHFAPTITFLESPTSDHH
WCIPFTVKGNPKPALQWFYNGAILNESKYICTKIHVTNHTEYHGCLQLDN
PTHMNNGDYTLIAKNEYGKDEKQISAHFMGWPGIDDGANPNYPDVIYEDY
GTAANDIGDTTNRSNEIPSTDVTDKTGREHLSVYAVVVIASVVGFCLLVM
LFLLKLARHSKFGMKDFSWFGFGKVKSRQGVGPASVISNDDDSASPLHHI
SNGSNTPSSSEGGPDAVIIGMTKIPVIENPQYVGITNSQLKPDTFVQHIK
RHNIVLKRELGEGAFGKVFLAECYNLCPEQDKILVAVKTLKDASDNARKD
FHREAELLTNLQHEHIVKFYGVCVEGDPLIMVFEYMKHGDLNKFLRAHGP
DAVLMAEGNPPTELTQSQMLHIAQQIAAGMVYLASQHFVHRDLATRNCLV
GENLLVKIGDFGMSRDVYSTDYYRVGGHTMLPIRWMPPESIMYRKFTTES
DVWSLGVVLWEIFTYGKQPWYQLSNNEVIECITQGRVLQRPRTCPQEVYE
LMLGCWQREPHMRKNIKGIHTLLQNLAKASPVYLDILG

Preferably, this embodiment of the first coding sequence comprises a nucleotide sequence referred to herein as SEQ ID NO. 12, or a fragment or variant thereof, as set out below:

[SEQ ID No: 12]
ATGTCGTCCTGGATAAGGTGGCATGGACCCGCCATGGCGCGGCTCTGGGG
CTTCTGCTGGCTGGTTGTGGGCTTCTGGAGGGCCGCTTTCGCCTGTCCCA
CGTCCTGCAAATGCAGTGCCTCTCGGATCTGGTGCAGCGACCCTTCTCCT
GGCATCGTGGCATTTCCGAGATTGGAGCCTAACAGTGTAGATCCTGAGAA
CATCACCGAAATTTTCATCGCAAACCAGAAAAGGTTAGAAATCATCAACG
AAGATGATGTTGAAGCTTATGTGGGACTGAGAAATCTGACAATTGTGGAT
TCTGGATTAAAATTTGTGGCTCATAAAGCATTTCTGAAAAACAGCAACCT
GCAGCACATCAATTTTACCCGAAACAAACTGACGAGTTTGTCTAGGAAAC
ATTTCCGTCACCTTGACTTGTCTGAACTGATCCTGGTGGGCAATCCATTT
ACATGCTCCTGTGACATTATGTGGATCAAGACTCTCCAAGAGGCTAAATC
CAGTCCAGACACTCAGGATTTGTACTGCCTGAATGAAAGCAGCAAGAATA
TTCCCCTGGCAAACCTGCAGATACCCAATTGTGGTTTGCCATCTGCAAAT
CTGGCCGCACCTAACCTCACTGTGGAGGAAGGAAAGTCTATCACATTATC
CTGTAGTGTGGCAGGTGATCCGGTTCCTAATATGTATTGGGATGTTGGTA
ACCTGGTTTCCAAACATATGAATGAAACAAGCCACACACAGGGCTCCTTA
AGGATAACTAACATTTCATCCGATGACAGTGGGAAGCAGATCTCTTGTGT
GGCGGAAAATCTTGTAGGAGAAGATCAAGATTCTGTCAACCTCACTGTGC
ATTTTGCACCAACTATCACATTTCTCGAATCTCCAACCTCAGACCACCAC
TGGTGCATTCCATTCACTGTGAAAGGCAACCCCAAACCAGCGCTTCAGTG
GTTCTATAACGGGGCAATATTGAATGAGTCCAAATACATCTGTACTAAAA
TACATGTTACCAATCACACGGAGTACCACGGCTGCCTCCAGCTGGATAAT
CCCACTCACATGAACAATGGGGACTACACTCTAATAGCCAAGAATGAGTA
TGGGAAGGATGAGAAACAGATTTCTGCTCACTTCATGGGCTGGCCTGGAA
TTGACGATGGTGCAAACCCAAATTATCCTGATGTAATTTATGAAGATTAT
GGAACTGCAGCGAATGACATCGGGGACACCACGAACAGAAGTAATGAAAT
CCCTTCCACAGACGTCACTGATAAAACCGGTCGGGAACATCTCTCGGTCT
ATGCTGTGGTGGTGATTGCGTCTGTGGTGGGATTTTGCCTTTTGGTAATG
CTGTTTCTGCTTAAGTTGGCAAGACACTCCAAGTTTGGCATGAAAGATTT
CTCATGGTTTGGATTTGGGAAAGTAAAATCAAGACAAGGTGTTGGCCCAG
CCTCCGTTATCAGCAATGATGATGACTCTGCCAGCCCACTCCATCACATC
TCCAATGGGAGTAACACTCCATCTTCTTCGGAAGGTGGCCCAGATGCTGT
CATTATTGGAATGACCAAGATCCCTGTCATTGAAAATCCCCAGTACTTTG
GCATCACCAACAGTCAGCTCAAGCCAGACACATTTGTTCAGCACATCAAG
CGACATAACATTGTTCTGAAAAGGGAGCTAGGCGAAGGAGCCTTTGGAAA
AGTGTTCCTAGCTGAATGCTATAACCTCTGTCCTGAGCAGGACAAGATCT
TGGTGGCAGTGAAGACCCTGAAGGATGCCAGTGACAATGCACGCAAGGAC
TTCCACCGTGAGGCCGAGCTCCTGACCAACCTCCAGCATGAGCACATCGT
CAAGTTCTATGGCGTCTGCGTGGAGGGCGACCCCCTCATCATGGTCTTTG
AGTACATGAAGCATGGGGACCTCAACAAGTTCCTCAGGGCACACGGCCCT
GATGCCGTGCTGATGGCTGAGGGCAACCCGCCCACGGAACTGACGCAGTC
GCAGATGCTGCATATAGCCCAGCAGATCGCCGCGGGCATGGTCTACCTGG
CGTCCCAGCACTTCGTGCACCGCGATTTGGCCACCAGGAACTGCCTGGTC
GGGGAGAACTTGCTGGTGAAAATCGGGGACTTTGGGATGTCCCGGGACGT
GTACAGCACTGACTACTACAGGGTCGGTGGCCACACAATGCTGCCCATTC
GCTGGATGCCTCCAGAGAGCATCATGTACAGGAAATTCACGACGGAAAGC
GACGTCTGGAGCCTGGGGGTCGTGTTGTGGGAGATTTTCACCTATGGCAA
ACAGCCCTGGTACCAGCTGTCAAACAATGAGGTGATAGAGTGTATCACTC
AGGGCCGAGTCCTGCAGCGACCCCGCACGTGCCCCCAGGAGGTGTATGAG
CTGATGCTGGGGTGCTGGCAGCGAGAGCCCCACATGAGGAAGAACATCAA
GGGCATCCATACCCTCCTTCAGAACTTGGCCAAGGCATCTCCGGTCTACC
TGGACATTCTAGGC

The inventors have spent considerable inventive endeavour in studying the sequence of the TrkB receptor and have realised that TrkB comprises five tyrosine residues (at position 516, 701, 705, 706 and 816 of SEQ ID No: 9), which are normally phosphorylated following dimerization and autophosphorylation in the presence of a BDNF dimer. A problem with phosphorylation of these five tyrosine residues is that the receptor can be readily deactivated by a phosphatase, such as the Shp-2 phosphatase. Accordingly, in order to prevent phosphorylation and resultant deactivation of the receptor in vivo, preferably one or more of these key tyrosines is mutated (more preferably, to glutamic acid) in order to mimic the resultant phosphotyrosine and produce a receptor which remains active in the presence of BDNF, and which cannot be deactivated by a phosphatise, such as the Shp-2 phosphatase. Such mutant forms of TrkB are aimed at producing TrkB receptor activity which remains active for longer periods, or until the receptor is internalised.

The DNA and amino acid sequences provided below illustrate the positions of these five tyrosine (Y) residues which have been mutated into five glutamic acid (E) residues. It will be appreciated that 1, 2, 3, 4 or 5 of these residues may be mutated to glutamic acid in embodiments of the invention. Various combinations of these mutations is also envisaged, e.g. positions 516 and 701 only, or positions 705, 706 and 816 only, and so on.

Accordingly, in another preferred embodiment, the first coding sequence comprises a nucleotide sequence encoding a mutant form of TrkB receptor, wherein one or more tyrosine residue at position 516, 701, 705, 706 and/or 816 of SEQ ID No: 9 is modified or mutated. Preferably, at least two, three or four tyrosine residues at position 516, 701, 705, 706 and/or 816 of SEQ ID No: 9 are modified. Most preferably, all five tyrosine residues at position 516, 701, 705, 706 and/or 816 of SEQ ID No: 9 are modified.

Preferably, the or each tyrosine residue is modified to a different amino acid residue, more preferably a glutamic acid. Thus, preferably the mutant form of the TrkB receptor comprises Y516E, Y701E, Y705E, Y706E and/or Y816E.

Preferably, the modified form of the TrkB receptor comprises an amino acid sequence referred to herein as SEQ ID NO. 13, or a fragment or variant thereof, as set out below:

[SEQ ID No: 13]
MSSWIRWHGPAMARLWGFCWLVVGFWRAAFACPTSCKCSASRIWCSDPSP
GIVAFPRLEPNSVDPENITEIFIANQKRLEIINEDDVEAYVGLRNLTIVD
SGLKFVAHKAFLKNSNLQHINFTRNKLTSLSRKHFRHLDLSELILVGNPF
TCSCDIMWIKTLQEAKSSPDTQDLYCLNESSKNIPLANLQIPNCGLPSAN
LAAPNLTVEEGKSITLSCSVAGDPVPNMYWDVGNLVSKHMNETSHTQGSL
RITNISSDDSGKQISCVAENLVGEDQDSVNLTVHFAPTITFLESPTSDHH
WCIPFTVKGNPKPALQWFYNGAILNESKYICTKIHVTNHTEYHGCLQLDN
PTHMNNGDYTLIAKNEYGKDEKQISAHFMGWPGIDDGANPNYPDVIYEDY
GTAANDIGDTTNRSNEIPSTDVTDKTGREHLSVYAVVVIASVVGFCLLVM
LFLLKLARHSKFGMKGPASVISNDDDSASPLHHISNGSNTPSSSEGGPDA
VIIGMTKIPVIENPQEFGITNSQLKPDTFVQHIKRHNIVLKRELGEGAFG
KVFLAECYNLCPEQDKILVAVKTLKDASDNARKDFHREAELLTNLQHEHI
VKFYGVCVEGDPLIMVFEYMKHGDLNKFLRAHGPDAVLMAEGNPPTELTQ
SQMLHIAQQIAAGMVYLASQHFVHRDLATRNCLVGENLLVKIGDFGMSRD
VESTDEERVGGHTMLPIRWMPPESIMYRKFTTESDVWSLGVVLWEIFTYG
KQPWYQLSNNEVIECITQGRVLQRPRTCPQEVYELMLGCWQREPHMRKNI
KGIHTLLQNLAKASPVELDILG

Preferably, in this embodiment, the first coding sequence comprises a nucleotide sequence referred to herein as SEQ ID NO. 14, or a fragment or variant thereof, as set out below:

[SEQ ID No: 14]
ATGTCGTCCTGGATAAGGTGGCATGGACCCGCCATGGCGCGGCTCTGGGG
CTTCTGCTGGCTGGTTGTGGGCTTCTGGAGGGCCGCTTTCGCCTGTCCCA
CGTCCTGCAAATGCAGTGCCTCTCGGATCTGGTGCAGCGACCCTTCTCCT
GGCATCGTGGCATTTCCGAGATTGGAGCCTAACAGTGTAGATCCTGAGAA
CATCACCGAAATTTTCATCGCAAACCAGAAAAGGTTAGAAATCATCAACG
AAGATGATGTTGAAGCTTATGTGGGACTGAGAAATCTGACAATTGTGGAT
TCTGGATTAAAATTTGTGGCTCATAAAGCATTTCTGAAAAACAGCAACCT
GCAGCACATCAATTTTACCCGAAACAAACTGACGAGTTTGTCTAGGAAAC
ATTTCCGTCACCTTGACTTGTCTGAACTGATCCTGGTGGGCAATCCATTT
ACATGCTCCTGTGACATTATGTGGATCAAGACTCTCCAAGAGGCTAAATC
CAGTCCAGACACTCAGGATTTGTACTGCCTGAATGAAAGCAGCAAGAATA
TTCCCCTGGCAAACCTGCAGATACCCAATTGTGGTTTGCCATCTGCAAAT
CTGGCCGCACCTAACCTCACTGTGGAGGAAGGAAAGTCTATCACATTATC
CTGTAGTGTGGCAGGTGATCCGGTTCCTAATATGTATTGGGATGTTGGTA
ACCTGGTTTCCAAACATATGAATGAAACAAGCCACACACAGGGCTCCTTA
AGGATAACTAACATTTCATCCGATGACAGTGGGAAGCAGATCTCTTGTGT
GGCGGAAAATCTTGTAGGAGAAGATCAAGATTCTGTCAACCTCACTGTGC
ATTTTGCACCAACTATCACATTTCTCGAATCTCCAACCTCAGACCACCAC
TGGTGCATTCCATTCACTGTGAAAGGCAACCCCAAACCAGCGCTTCAGTG
GTTCTATAACGGGGCAATATTGAATGAGTCCAAATACATCTGTACTAAAA
TACATGTTACCAATCACACGGAGTACCACGGCTGCCTCCAGCTGGATAAT
CCCACTCACATGAACAATGGGGACTACACTCTAATAGCCAAGAATGAGTA
TGGGAAGGATGAGAAACAGATTTCTGCTCACTTCATGGGCTGGCCTGGAA
TTGACGATGGTGCAAACCCAAATTATCCTGATGTAATTTATGAAGATTAT
GGAACTGCAGCGAATGACATCGGGGACACCACGAACAGAAGTAATGAAAT
CCCTTCCACAGACGTCACTGATAAAACCGGTCGGGAACATCTCTCGGTCT
ATGCTGTGGTGGTGATTGCGTCTGTGGTGGGATTTTGCCTTTTGGTAATG
CTGTTTCTGCTTAAGTTGGCAAGACACTCCAAGTTTGGCATGAAAGGCCC
AGCCTCCGTTATCAGCAATGATGATGACTCTGCCAGCCCACTCCATCACA
TCTCCAATGGGAGTAACACTCCATCTTCTTCGGAAGGTGGCCCAGATGCT
GTCATTATTGGAATGACCAAGATCCCTGTCATTGAAAATCCCCAGGAATT
TGGCATCACCAACAGTCAGCTCAAGCCAGACACATTTGTTCAGCACATCA
AGCGACATAACATTGTTCTGAAAAGGGAGCTAGGCGAAGGAGCCTTTGGA
AAAGTGTTCCTAGCTGAATGCTATAACCTCTGTCCTGAGCAGGACAAGAT
CTTGGTGGCAGTGAAGACCCTGAAGGATGCCAGTGACAATGCACGCAAGG
ACTTCCACCGTGAGGCCGAGCTCCTGACCAACCTCCAGCATGAGCACATC
GTCAAGTTCTATGGCGTCTGCGTGGAGGGCGACCCCCTCATCATGGTCTT
TGAGTACATGAAGCATGGGGACCTCAACAAGTTCCTCAGGGCACACGGCC
CTGATGCCGTGCTGATGGCTGAGGGCAACCCGCCCACGGAACTGACGCAG
TCGCAGATGCTGCATATAGCCCAGCAGATCGCCGCGGGCATGGTCTACCT
GGCGTCCCAGCACTTCGTGCACCGCGATTTGGCCACCAGGAACTGCCTGG
TCGGGGAGAACTTGCTGGTGAAAATCGGGGACTTTGGGATGTCCCGGGAC
GTGGAAAGCACTGACGAAGAAAGGGTCGGTGGCCACACAATGCTGCCCAT
TCGCTGGATGCCTCCAGAGAGCATCATGTACAGGAAATTCACGACGGAAA
GCGACGTCTGGAGCCTGGGGGTCGTGTTGTGGGAGATTTTCACCTATGGC
AAACAGCCCTGGTACCAGCTGTCAAACAATGAGGTGATAGAGTGTATCAC
TCAGGGCCGAGTCCTGCAGCGACCCCGCACGTGCCCCCAGGAGGTGTATG
AGCTGATGCTGGGGTGCTGGCAGCGAGAGCCCCACATGAGGAAGAACATC
AAGGGCATCCATACCCTCCTTCAGAACTTGGCCAAGGCATCTCCGGTCGA
ACTGGACATTCTAGGC

It will be appreciated that the second coding sequence encodes an agonist of the TrkB receptor, which is preferably a member of the neurotrophin family of trophic factors. The agonist of the TrkB receptor may be a member of the neurotrophin family of trophic factors lacking the pro-sequence. The agonist of the TrkB receptor may be a member of the neurotrophin family of trophic factors in the mature form. Preferred agonists of the TrkB receptor may therefore be selected from a group of agonists consisting of: Brain-derived neurotrophic factor (BDNF); nerve growth factor (NGF); neurotrophin-3 (NT-3); neurotrophin-4 (NT-4); and neurotrophin-5 (NT-5); or fragments thereof. Preferred agonists of the TrkB receptor may be selected from a group of agonists consisting of: Brain-derived neurotrophic factor (BDNF) lacking the pro-sequence; nerve growth factor (NGF) lacking the pro-sequence; neurotrophin-3 (NT-3) lacking the pro-sequence; neurotrophin-4 (NT-4) lacking the pro-sequence; and neurotrophin-5 (NT-5) lacking the pro-sequence; or fragments thereof. Preferred agonists of the TrkB receptor may be selected from a group of agonists consisting of: mature Brain-derived neurotrophic factor (BDNF); mature nerve growth factor (NGF); mature neurotrophin-3 (NT-3); mature neurotrophin-4 (NT-4); and mature neurotrophin-5 (NT-5); or fragments thereof.

The nucleotide and amino acid sequences of each of these agonists will be known to the skilled person. However, by way of example, the amino acid sequence of one embodiment of Neurotrophin-4 (NT-4) is substantially as set out in SEQ ID NO. 49, as follows:

[SEQ ID No: 49]
MLPLPSCSLPILLLFLLPSVPIESQPPPSTLPPFLAPEWDLLSPRVVLSR
GAPAGPPLLFLLEAGAFRESAGAPANRSRRGVSETAPASRRGELAVCDAV
SGWVTDRRTAVDLRGREVEVLGEVPAAGGSPLRQYFFETRCKADNAEEGG
PGAGGGGCRGVDRRHWVSECKAKQSYVRALTADAQGRVGWRWIRIDTACV
CTLLSRTGRA

The nucleic acid coding sequence of this embodiment of Neurotrophin-4 (NT-4) is substantially as set out in SEQ ID NO. 50, as follows:

[SEQ ID No: 50]
ATGCTCCCTCTCCCCTCATGCTCCCTCCCCATCCTCCTCCTTTTCCTCCT
CCCCAGTGTGCCAATTGAGTCCCAACCCCCACCCTCAACATTGCCCCCTT
TTCTGGCCCCTGAGTGGGACCTTCTCTCCCCCCGAGTAGTCCTGTCTAGG
GGTGCCCCTGCTGGGCCCCCTCTGCTCTTCCTGCTGGAGGCTGGGGCCTT
TCGGGAGTCAGCAGGTGCCCCGGCCAACCGCAGCCGGCGTGGGGTGAGCG
AAACTGCACCAGCGAGTCGTCGGGGTGAGCTGGCTGTGTGCGATGCAGTC
AGTGGCTGGGTGACAGACCGCCGGACCGCTGTGGACTTGCGTGGGCGCGA
GGTGGAGGTGTTGGGCGAGGTGCCTGCAGCTGGCGGCAGTCCCCTCCGCC
AGTACTTCTTTGAAACCCGCTGCAAGGCTGATAACGCTGAGGAAGGTGGC
CCGGGGGCAGGTGGAGGGGGCTGCCGGGGAGTGGACAGGAGGCACTGGGT
ATCTGAGTGCAAGGCCAAGCAGTCCTATGTGCGGGCATTGACCGCTGATG
CCCAGGGCCGTGTGGGCTGGCGATGGATTCGAATTGACACTGCCTGCGTC
TGCACACTCCTCAGCCGGACTGGCCGGGCC

The amino acid sequence of the signal peptide for the NT-4 sequence is substantially as set out in SEQ ID NO. 51, as follows:

[SEQ ID No: 51]
MLPLPSCSLPILLLFLLPSVPIES

The nucleic acid sequence of this signal peptide is substantially as set out in SEQ ID NO. 52, as follows:

[SEQ ID No: 52]
ATGCTCCCTCTCCCCTCATGCTCCCTCCCCATCCTCCTCCTTTTCCTCCT
CCCCAGTGTGCCAATTGAGTCC

The amino acid sequence of the propeptide for this NT-4 sequence is substantially as set out in SEQ ID NO. 53, as follows:

[SEQ ID No: 53]
QPPPSTLPPFLAPEWDLLSPRVVLSRGAPAGPPLLFLLEAGAFRESAGAP
ANRSRR

The nucleic acid sequence of this propeptide is substantially as set out in SEQ ID NO. 54, as follows:

[SEQ ID No: 54]
CAACCCCCACCCTCAACATTGCCCCCTTTTCTGGCCCCTGAGTGGGACCT
TCTCTCCCCCCGAGTAGTCCTGTCTAGGGGTGCCCCTGCTGGGCCCCCTC
TGCTCTTCCTGCTGGAGGCTGGGGCCTTTCGGGAGTCAGCAGGTGCCCCG
GCCAACCGCAGCCGGCGT

The amino acid sequence of the mature protein sequence for this NT-4 sequence is substantially as set out in SEQ ID NO. 55, as follows:

[SEQ ID No: 55]
GVSETAPASRRGELAVCDAVSGWVTDRRTAVDLRGREVEVLGEVPAAGGS
PLRQYFFETRCKADNAEEGGPGAGGGGCRGVDRRHWVSECKAKQSYVRAL
TADAQGRVGWRWIRIDTACVCTLLSRTGRA

The nucleic acid coding sequence of this mature NT-4 protein is substantially as set out in SEQ ID NO. 56, as follows:

[SEQ ID No: 56]
GGGGTGAGCGAAACTGCACCAGCGAGTCGTCGGGGTGAGCTGGCTGTGTG
CGATGCAGTCAGTGGCTGGGTGACAGACCGCCGGACCGCTGTGGACTTGC
GTGGGCGCGAGGTGGAGGTGTTGGGCGAGGTGCCTGCAGCTGGCGGCAGT
CCCCTCCGCCAGTACTTCTTTGAAACCCGCTGCAAGGCTGATAACGCTGA
GGAAGGTGGCCCGGGGGCAGGTGGAGGGGGCTGCCGGGGAGTGGACAGGA
GGCACTGGGTATCTGAGTGCAAGGCCAAGCAGTCCTATGTGCGGGCATTG
ACCGCTGATGCCCAGGGCCGTGTGGGCTGGCGATGGATTCGAATTGACAC
TGCCTGCGTCTGCACACTCCTCAGCCGGACTGGCCGGGCC

Accordingly, in one preferred embodiment, the second coding sequence encodes neurotrophin-4 (NT-4), which may comprise an amino acid sequence substantially as set out in SEQ ID NO: 49 or 55, or fragment or variant thereof. Thus, the second coding sequence may comprise a nucleotide sequence substantially as set out in SEQ ID No: 50 or 56, or a fragment or variant thereof.

Most preferred agonists of the TrkB receptor, however, include prepro-brain derived neurotrophic factor (pre-pro-BDNF), pro-BDNF or mature BDNF (mBDNF). BDNF is initially synthesised as the precursor protein, preproBDNF, by ribosomes found on endoplasmic reticulum. There are at least 17 known splice variants encoded by the human preproBDNF gene (ENSG00000176697). Once preproBDNF has entered into the rough endoplasmic reticulum, preproBDNF is converted into proBDNF by cleavage of the signal peptide (i.e. the “pre” sequence). proBDNF is converted into mBDNF by cleavage of an additional N-terminal peptide sequence that is present on proBDNF.

Both proBDNF and mBDNF are then secreted into the extracellular space, where they bind to and activate receptors on various cells.

proBDNF preferentially binds to and activates the receptor, p75^{NTR}, which, when activated, can induce apoptosis in some cell types. Thus, in one preferred embodiment, proBDNF is an agonist of the p75^{NTR} receptor. In one embodiment, the proBDNF is canonical proBDNF. Preferably, canonical proBDNF comprises an amino acid sequence referred to herein as SEQ ID NO. 15, or a fragment or variant thereof, as set out below:

[SEQ ID No: 15]
APMKEANIRGQGGLAYPGVRTHGTLESVNGPKAGSRGLTSLADTFEHVIE
ELLDEDQKVRPNEENNKDADLYTSRVMLSSQVPLEPPLLFLLEEYKNYLD
AANMSMRVRRHSDPARRGELSVCDSISEWVTAADKKTAVDMSGGTVTVLE
KVPVSKGQLKQYFYETKCNPMGYTKEGCRGIDKRHWNSQCRTTQSYVRAL
TMDSKKRIGWRFIRIDTSCVCTLTIKRGR

Preferably, in this embodiment, the second coding sequence comprises a nucleotide sequence referred to herein as SEQ ID NO. 16, or a fragment or variant thereof, as set out below:

[SEQ ID No: 16]
GCCCCCATGAAAGAAGCAAACATCCGAGGACAAGGTGGCTTGGCCTACCC
AGGTGTGCGGACCCATGGGACTCTGGAGAGCGTGAATGGGCCCAAGGCAG
GTTCAAGAGGCTTGACATCATTGGCTGACACTTTCGAACACGTGATAGAA
GAGCTGTTGGATGAGGACCAGAAAGTTCGGCCCAATGAAGAAAACAATAA
GGACGCAGACTTGTACACGTCCAGGGTGATGCTCAGTAGTCAAGTGCCTT
TGGAGCCTCCTCTTCTCTTTCTGCTGGAGGAATACAAAAATTACCTAGAT
GCTGCAAACATGTCCATGAGGGTCCGGCGCCACTCTGACCCTGCCCGCCG
AGGGGAGCTGAGCGTGTGTGACAGTATTAGTGAGTGGGTAACGGCGGCAG
ACAAAAAGACTGCAGTGGACATGTCGGGCGGGACGGTCACAGTCCTTGAA
AAGGTCCCTGTATCAAAAGGCCAACTGAAGCAATACTTCTACGAGACCAA
GTGCAATCCCATGGGTTACACAAAAGAAGGCTGCAGGGGCATAGACAAAA
GGCATTGGAACTCCCAGTGCCGAACTACCCAGTCGTACGTGCGGGCCCTT
ACCATGGATAGCAAAAAGAGAATTGGCTGGCGATTCATAAGGATAGACAC
TTCTTGTGTATGTACATTGACCATTAAAAGGGGAAGATAG

In another embodiment, the proBDNF is isoform 2 of proBDNF, which preferably comprises a Valine to Methionione mutation (amino acid underlined). Preferably, isoform 2 of proBDNF comprises an amino acid sequence referred to herein as SEQ ID NO. 17, or a fragment or variant thereof, as set out below:

[SEQ ID No: 17]
APMKEANIRGQGGLAYPGVRTHGTLESVNGPKAGSRGLTSLADTFEHMIE
ELLDEDQKVRPNEENNKDADLYTSRVMLSSQVPLEPPLLFLLEEYKNYLD
AANMSMRVRRHSDPARRGELSVCDSISEWVTAADKKTAVDMSGGTVTVLE
KVPVSKGQLKQYFYETKCNPMGYTKEGCRGIDKRHWNSQCRTTQSYVRAL
TMDSKKRIGWRFIRIDTSCVCTLTIKRGR

In one embodiment, however, the agonist is not proBDNF, or a fragment or variant thereof, but instead the second coding sequence preferably comprises a nucleotide sequence which encodes mature BDNF. Mature BDNF (mBDNF) preferentially binds to, and activates, TrkB, which, when activated, promotes survival of nerve cells. Thus, mature BDNF is a most preferred agonist of TrkB. The construct according to the first aspect is advantageous because, unlike other known genetic constructs, the construct is capable of producing mature BDNF protein, which has not been mis-folded.

Thus, in one preferred embodiment, the second coding sequence comprises a nucleotide sequence which encodes mature BDNF. mBDNF is common to all 17 isoforms encoded by the gene. There 7 protein different sequences, five of which have extended signal sequences to the canonical form, and one has a canonical signal sequence, but a Valine to Methionine mutation (which is common to isoforms 2, 4, 7, 8, 9, 10, 11, 12, 13, 14 and 16). It is believed that the valine to methionine mutation reduces release of BDNF from the cell.

Preferably, mature BDNF comprises an amino acid sequence referred to herein as SEQ ID NO. 18, or a fragment or variant thereof, as set out below:

[SEQ ID No: 18]
HSDPARRGELSVCDSISEWVTAADKKTAVDMSGGTVTVLEKVPVSKGQLK
QYFYETKCNPMGYTKEGCRGIDKRHWNSQCRTTQSYVRALTMDSKKRIGW
RFIRIDTSCVCTLTIKRGR

Preferably, this embodiment of the second coding sequence comprises a nucleotide sequence referred to herein as SEQ ID NO. 19, or a fragment or variant thereof, as set out below:

[SEQ ID No: 19]
ATGACCATCCTTTTCCTTACTATGGTTATTTCATACTTTGGTTGCATGAA
GGCTGCCCCCATGAAAGAAGCAAACATCCGAGGACAAGGTGGCTTGGCCT
ACCCAGGTGTGCGGACCCATGGGACTCTGGAGAGCGTGAATGGGCCCAAG
GCAGGTTCAAGAGGCTTGACATCATTGGCTGACACTTTCGAACACGTGAT
AGAAGAGCTGTTGGATGAGGACCAGAAAGTTCGGCCCAATGAAGAAAACA
ATAAGGACGCAGACTTGTACACGTCCAGGGTGATGCTCAGTAGTCAAGTG
CCTTTGGAGCCTCCTCTTCTCTTTCTGCTGGAGGAATACAAAAATTACCT
AGATGCTGCAAACATGTCCATGAGGGTCCGGCGCCACTCTGACCCTGCCC
GCCGAGGGGAGCTGAGCGTGTGTGACAGTATTAGTGAGTGGGTAACGGCG
GCAGACAAAAAGACTGCAGTGGACATGTCGGGCGGGACGGTCACAGTCCT
TGAAAAGGTCCCTGTATCAAAAGGCCAACTGAAGCAATACTTCTACGAGA
CCAAGTGCAATCCCATGGGTTACACAAAAGAAGGCTGCAGGGGCATAGAC
AAAAGGCATTGGAACTCCCAGTGCCGAACTACCCAGTCGTACGTGCGGGC
CCTTACCATGGATAGCAAAAAGAGAATTGGCTGGCGATTCATAAGGATAG
ACACTTCTTGTGTATGTACATTGACCATTAAAAGGGGAAGATAG

In another embodiment, the agonist is member of the neurotrophin family of trophic factors lacking the pro-sequence but with a signal peptide conjugated to the N-terminus. The agonist may be any member of the neurotrophin family of trophic factors in the mature form and with a signal peptide conjugated to the N-terminus. The signal peptide may be any signal peptide that promotes the proper folding or production of the agonist. In preferred embodiments, the signal peptide may be any signal peptide disclosed herein.

In yet another preferred embodiment, the agonist is mBDNF with a signal peptide conjugated to its N-terminus. As discussed below, the signal peptide may be canonical signal peptide of preproBDNF, or the signal peptide of IL-2, or a de novo novel signal sequence created by the inventors.

Preferably, the second coding sequence comprises a nucleotide sequence encoding a signal peptide for the agonist of the TrkB receptor, most preferably a signal peptide for BDNF. In one preferred embodiment, the nucleotide sequence encodes the canonical signal peptide for BDNF. Preferably, this embodiment of the second coding sequence comprises a nucleotide sequence which encodes a signal peptide comprising an amino acid sequence referred to herein as SEQ ID NO. 20, or a fragment or variant thereof, as set out below:

[SEQ ID No: 20]
MTILFLTMVISYFGCMKA

Preferably, this embodiment of the second coding sequence comprises a nucleotide sequence referred to herein as SEQ ID NO. 21, or a fragment or variant thereof, as set out below:

[SEQ ID No: 21]
ATGACCATCCTTTTCCTTACTATGGTTATTTCATACTTCGGTTGCATGAA
GGCG

The inventors have created a series of extended signal peptides. In preferred embodiments, the nucleotide sequence encoding an isoform signal peptide for BDNF is selected from the group consisting of: isoform 2, 3, 6, 5 and 4. The nucleic acid and amino acid sequences for each of these extended signal peptides are set out below.

Isoform 2
[SEQ ID No: 22]
MFHQVRRVMTILFLTMVISYFGCMKA
[SEQ ID No: 23]
ATGTTCCACCAGGTGAGAAGAGTGATGACCATCCTTTTCCTTACTATGGT
TATTTCATACTTCGGTTGCATGAAGGCG
Isoform 3 and 6
[SEQ ID No: 24]
MQSREEEWFHQVRRVMTILFLTMVISYFGCMKA
[SEQ ID No: 25]
ATGCAGAGCCGGGAAGAGGAATGGTTCCACCAGGTGAGAAGAGTGATGAC
CATCCTTTTCCTTACTATGGTTATTTCATACTTCGGTTGCATGAAGGCG
Isoform 5
[SEQ ID No: 26]
MLCAISLCARVRKLRSAGRCGKFHQVRRVMTILFLTMVISYFGCMKA
[SEQ ID No: 27]
ATGCTCTGTGCGATTTCATTGTGTGCTCGCGTTCGCAAGCTCCGTAGTGC
AGGAAGGTGCGGGAAGTTCCACCAGGTGAGAAGAGTGATGACCATCCTTT
TCCTTACTATGGTTATTTCATACTTCGGTTGCATGAAGGCG
Isoform 4
[SEQ ID No: 28]
MCGATSFLHECTRLILVTTQNAEFLQKGLQVHTCFGVYPHASVWHDCASQ
KKGCAVYLHVSVEFNKLIPENGFIKFHQVRRVMTILFLTMVISYFGCMKA
[SEQ ID No: 29]
ATGTGTGGAGCCACCAGTTTTCTCCATGAGTGCACAAGGTTAATCCTTGT
TACTACTCAGAATGCTGAGTTTCTACAGAAAGGGTTGCAGGTCCACACAT
GTTTTGGCGTCTACCCACACGCTTCTGTATGGCATGACTGTGCATCCCAG
AAGAAGGGCTGTGCTGTGTACCTCCACGTTTCAGTGGAATTTAACAAACT
GATCCCTGAAAATGGTTTCATAAAGTTCCACCAGGTGAGAAGAGTGATGA
CCATCCTTTTCCTTACTATGGTTATTTCATACTTCGGTTGCATGAAGGCG

Accordingly, in preferred embodiments, the second coding sequence comprises a nucleotide sequence encoding a signal sequence peptide referred to herein as any one of SEQ ID NO. 23, 25, 27 or 29. Preferably, the signal peptide comprises an amino acid sequence referred to herein as any one of SEQ ID NO. 22, 24, 26 or 28.

The inventors have also created various embodiments of novel signal peptides for the agonist, preferably BDNF. These signal peptides increase the level of basicity of the N-terminal section (with added lysine (K) and arginine (R) residues) and the proceeding hydrophobic region (with additions of leucine (L) residues), which increase secretion of BDNF compared to levels observed with the wild-type canonical signal sequence.

a) QTA003P (IL-2 signal)
[SEQ ID No: 30]
MYRMQLLSCIALSLALVTNS
[SEQ ID No: 31]
ATGTACAGGATGCAACTCCTGTCTTGCATTGCACTAAGTCTTGCACTTGT
CACAAACAGT
b) QTA004P
[SEQ ID No: 32]
MKRRVMIILFLTMVISYFGCMK
[SEQ ID No: 33]
ATGAAAAGAAGAGTGATGATCATCCTTTTCCTTACTATGGTTATTTCATA
CTTCGGTTGCATGAAGAGCG
c) QTA009P (modified IL-2)
[SEQ ID No: 34]
MRRMQLLLLIALSLALVTNS
[SEQ ID No: 35]
ATGAGGAGGATGCAACTCCTGCTCCTGATTGCACTAAGTCTTGCACTTGT
CACAAACAGT
d) QTA010P
[SEQ ID No: 36]
MRRMQLLLLTMVISYFGCMKA
[SEQ ID No: 37]
ATGAGGAGGATGCAACTCCTGCTCCTGACTATGGTTATTTCATACTTCGG
TTGCATGAAGGCG
e) QTA0012P
[SEQ ID No: 38]
MRILLLTMVISYFGCMKA
[SEQ ID No: 39]
ATGAGAATCCTTCTTCTTACTATGGTTATTTCATACTTCGGTTGCATGAA
GGCG
f) QTA0013P
[SEQ ID No: 40]
MRRILFLTMVISYFGCMKA
[SEQ ID No: 41]
ATGAGAAGAATCCTTTTCCTTACTATGGTTATTTCATACTTCGGTTGCAT
GAAGGCG
g) QTA0014P
[SEQ ID No: 42]
MRRFLFLLVISYFGCMKA
[SEQ ID No: 43]
ATGAGGAGGTTCCTTTTCCTTCTTGTTATTTCATACTTCGGTTGCATGAA
GGCG
i) QTA0015P
[SEQ ID No: 44]
MRRFLFLLYFGCMKA
[SEQ ID No: 45]
ATGAGGAGGTTCCTTTTCCTTCTTTACTTCGGTTGCATGAAGGCG

FIG. 6 shows nucleotide and amino acid sequences for further preferred embodiments of signal peptide used in the construct of the invention to boost secretion of the agonist, preferably BDNF. The second residue in the signal peptide is threonine (T) which is preferably replaced by one or more basic residue, such as lysine (K) or arginine (R). The next stretch of residues in the signal peptide including isoleucine (I), leucine (L), phenylalanine (F) and Leucine (L) is preferably replaced by one or more hydrophobic residues.

Accordingly, in preferred embodiments, the second coding sequence comprises a nucleotide sequence encoding a signal sequence peptide referred to herein as any one of SEQ ID NO. 31, 33, 35, 37, 39, 41, 43, 45, 61, 63, 65, 67, 69, 71, 73, 75, 77, 79, 81, 83, 85, 87, 89, 91, 93, 95, 97, 99, 101 or 103. Preferably, the signal peptide comprises an amino acid sequence referred to herein as any one of SEQ ID NO. 30, 32, 34, 36, 38, 40, 42, 44, 60, 62, 64, 66, 68, 70, 72, 74, 76, 78, 80, 82, 84, 86, 88, 90, 92, 94, 96, 98, 100 or 102.

Accordingly, it will be appreciated that the inventors have modified the BDNF gene sequence by removal of the pro-sequence, which also has never been achieved before, with the result of generated properly folded mature BDNF, combined with the introduction of completely novel signal peptides, which significantly boost BDNF production and release above that ever achieved with the endogenous sequence.

Preferably, the genetic construct comprises left and/or right Inverted Terminal Repeat sequences (ITRs). Preferably, each ITR is disposed at the 5′ and/or 3′ end of the construct. An ITR can be specific to a virus (e.g. AAV or lentivirus) serotype, and can be any sequence, so long as it forms a hairpin loop in its secondary structure.

The DNA sequence of one embodiment (left ITR from a commercially available AAV plasmid) of the ITR is represented herein as SEQ ID No: 46, as follows:

[SEQ ID NO: 46]
CCTGCAGGCAGCTGCGCGCTCGCTCGCTCACTGAGGCCGCCCGGGCGTCG
GGCGACCTTTGGTCGCCCGGCCTCAGTGAGCGAGCGAGCGCGCAGAGAGG
GAGTGGCCAACTCCATCACTAGGGGTTCCT

The DNA sequence of another embodiment (right ITR from a commercially available AAV plasmid) of the ITR is represented herein as SEQ ID No: 47, as follows:

[SEQ ID NO: 47]
AGGAACCCCTAGTGATGGAGTTGGCCACTCCCTCTCTGCGCGCTCGCTCG
CTCACTGAGGCCGGGCGACCAAAGGTCGCCCGACGCCCGGGCTTTGCCCG
GGCGGCCTCAGTGAGCGAGCGAGCGCGCAGCTGCCTGCAGG

From the foregoing, the skilled person will appreciate the nucleotide sequence of an embodiment of the construct of the first aspect, as well as the amino acid sequence of the encoded transgene. However, for the avoidance of doubt, the coding sequence of codon optimised 2940 bp sequence for murine TrkB receptor-viral-2A peptide-mBDNF contained within the plasmid QTA020P (and the vector QTA020V), is referred to here as SEQ ID No: 107, as follows:

[SEQ ID No: 107]
ATGAGCCCATGGCTGAAGTGGCACGGACCAGCAATGGCAAGACTGTGGGG
CCTGTGCCTGCTGGTGCTGGGCTTCTGGAGAGCCAGCCTGGCCTGTCCAA
CCTCCTGCAAGTGTAGCTCCGCCAGGATCTGGTGCACAGAGCCTTCTCCA
GGCATCGTGGCCTTTCCCCGCCTGGAGCCTAACAGCGTGGATCCCGAGAA
TATCACCGAGATCCTGATCGCCAACCAGAAGCGGCTGGAGATCATCAATG
AGGACGATGTGGAGGCCTACGTGGGCCTGAGAAACCTGACAATCGTGGAC
TCCGGCCTGAAGTTCGTGGCCTATAAGGCCTTTCTGAAGAACTCTAATCT
GAGGCACATCAACTTCACCCGCAATAAGCTGACATCTCTGAGCCGGAGAC
ACTTTCGGCACCTGGATCTGTCCGACCTGATCCTGACCGGCAATCCATTC
ACATGCTCTTGTGACATCATGTGGCTGAAGACCCTGCAGGAGACAAAGTC
TAGCCCCGATACCCAGGACCTGTACTGTCTGAACGAGTCCTCTAAGAATA
TGCCTCTGGCCAACCTGCAGATCCCTAATTGTGGACTGCCAAGCGCCCGG
CTGGCCGCACCTAACCTGACAGTGGAGGAGGGCAAGTCCGTGACACTGTC
CTGTTCTGTGGGCGGCGATCCCCTGCCTACCCTGTATTGGGACGTGGGCA
ACCTGGTGTCTAAGCACATGAATGAGACCTCCCACACACAGGGCTCTCTG
AGAATCACAAATATCAGCTCCGACGATAGCGGCAAGCAGATCTCTTGCGT
GGCAGAGAACCTGGTGGGAGAGGATCAGGACAGCGTGAATCTGACCGTGC
ACTTCGCCCCCACCATCACATTTCTGGAGTCTCCTACCAGCGATCACCAC
TGGTGCATCCCCTTCACAGTGCGGGGAAACCCAAAGCCCGCCCTGCAGTG
GTTTTACAACGGCGCCATCCTGAATGAGTCCAAGTATATCTGTACCAAGA
TCCACGTGACCAACCACACAGAGTACCACGGCTGCCTGCAGCTGGATAAT
CCCACCCACATGAACAATGGCGACTACACACTGATGGCCAAGAACGAGTA
TGGCAAGGACGAGAGGCAGATCAGCGCCCACTTCATGGGCCGCCCTGGAG
TGGATTATGAGACCAACCCTAATTACCCAGAGGTGCTGTATGAGGACTGG
ACCACACCTACCGATATCGGCGACACCACAAACAAGTCTAATGAGATCCC
AAGCACAGATGTGGCCGACCAGTCTAACAGGGAGCACCTGAGCGTGTACG
CAGTGGTGGTCATCGCCTCCGTGGTGGGCTTCTGCCTGCTGGTCATGCTG
CTGCTGCTGAAGCTGGCCCGCCACTCTAAGTTTGGCATGAAGGGCCCAGC
CTCCGTGATCTCTAATGACGATGACAGCGCCAGCCCCCTGCACCACATCA
GCAACGGCTCCAATACCCCTTCTAGCTCCGAGGGCGGCCCAGATGCCGTG
ATCATCGGCATGACAAAGATCCCCGTGATCGAGAACCCTCAGTACTTCGG
CATCACCAATTCCCAGCTGAAGCCTGACACATTTGTGCAGCACATCAAGC
GGCACAACATCGTGCTGAAGAGGGAACTGGGAGAGGGAGCCTTCGGCAAG
GTGTTTCTGGCCGAGTGCTATAACCTGTGCCCAGAGCAGGATAAGATCCT
GGTGGCCGTGAAGACCCTGAAGGATGCCAGCGACAACGCCCGGAAGGACT
TCCACAGAGAGGCCGAGCTGCTGACAAATCTGCAGCACGAGCACATCGTG
AAGTTTTACGGCGTGTGCGTGGAGGGCGACCCTCTGATCATGGTGTTCGA
GTATATGAAGCACGGCGATCTGAACAAGTTTCTGAGAGCACACGGACCAG
ATGCCGTGCTGATGGCAGAGGGAAATCCCCCTACCGAGCTGACACAGTCT
CAGATGCTGCACATTGCACAGCAGATTGCAGCAGGAATGGTGTACCTGGC
CAGCCAGCACTTCGTGCACAGGGATCTGGCAACCAGAAACTGCCTGGTGG
GAGAGAATCTGCTGGTGAAGATCGGCGACTTTGGCATGTCCCGGGACGTG
TACTCTACCGACTACTATAGAGTGGGCGGCCACACAATGCTGCCCATCAG
GTGGATGCCACCCGAGAGCATCATGTATCGCAAGTTCACCACAGAGTCTG
ACGTGTGGAGCCTGGGCGTGGTGCTGTGGGAGATCTTTACCTACGGCAAG
CAGCCTTGGTATCAGCTGTCCAACAATGAAGTGATCGAGTGTATTACACA
GGGACGCGTGCTGCAGAGGCCACGCACATGCCCCCAGGAGGTGTACGAGC
TGATGCTGGGCTGTTGGCAGCGGGAGCCACACACCAGAAAGAACATCAAG
AGCATCCACACACTGCTGCAGAATCTGGCCAAGGCCTCCCCCGTGTATCT
GGACATCCTGGGCAGCGGAGCTACTAACTTCAGCCTGCTGAAGCAGGCTG
GAGACGTGGAGGAGAACCCTGGACCTATGAGAATCCTTCTTCTTACTATG
GTTATTTCATACTTCGGTTGCATGAAGGCGCACTCCGACCCTGCCCGCCG
TGGGGAGCTGAGCGTGTGTGACAGTATTAGCGAGTGGGTCACAGCGGCAG
ATAAAAAGACTGCAGTGGACATGTCTGGCGGGACGGTCACAGTCCTAGAG
AAAGTCCCGGTATCCAAAGGCCAACTGAAGCAGTATTTCTACGAGACCAA
GTGTAATCCCATGGGTTACACCAAGGAAGGCTGCAGGGGCATAGACAAAA
GGCACTGGAACTCGCAATGCCGAACTACCCAATCGTATGTTCGGGCCCTT
ACTATGGATAGCAAAAAGAGAATTGGCTGGCGATTCATAAGGATAGACAC
TTCCTGTGTATGTACACTGACCATTAAAAGGGGAAGATAG

The coding sequence of codon optimised 2943 bp sequence for human TrkB receptor-viral-2A peptide-mBDNF contained within the plasmid QTA029P (and the vector QTA029V), is referred to here as SEQ ID No: 108, as follows:

[SEQ ID No: 108]
ATGTCATCTTGGATCCGCTGGCACGGGCCAGCGATGGCCCGATTGTGGGG
CTTCTGCTGGCTTGTTGTAGGCTTCTGGCGCGCGGCGTTCGCGTGTCCGA
CCTCTTGCAAATGCTCAGCAAGCCGAATTTGGTGCTCAGACCCTAGTCCA
GGAATTGTTGCATTCCCCCGACTGGAACCAAACTCCGTCGACCCGGAGAA
TATAACTGAGATATTTATTGCAAATCAAAAACGCCTTGAAATCATTAACG
AGGATGACGTGGAGGCCTACGTTGGTTTGAGAAATCTTACTATTGTCGAC
TCCGGACTTAAATTTGTAGCTCATAAAGCCTTCCTGAAGAACTCTAATCT
GCAGCACATTAATTTCACGAGAAATAAGCTGACCAGCTTGTCCCGGAAGC
ATTTCCGCCATCTCGACCTGAGCGAGCTCATACTGGTCGGAAACCCATTT
ACGTGCTCCTGTGACATCATGTGGATCAAAACTCTGCAAGAGGCGAAAAG
TAGTCCGGATACCCAAGACCTTTACTGTCTTAATGAAAGCTCAAAAAATA
TCCCGCTGGCCAACCTGCAGATACCGAACTGCGGACTTCCTAGTGCGAAT
TTGGCTGCCCCAAATCTTACCGTCGAAGAAGGCAAATCAATCACGCTTTC
TTGTTCTGTAGCTGGAGATCCAGTGCCTAATATGTATTGGGACGTGGGTA
ACCTCGTCTCAAAACATATGAACGAAACGAGCCACACCCAGGGCTCTTTG
CGGATAACAAACATCTCCTCTGATGATTCTGGAAAGCAAATCAGTTGCGT
AGCTGAAAATCTGGTTGGCGAAGATCAAGATTCAGTCAATCTGACAGTCC
ATTTCGCCCCAACGATCACCTTTCTGGAGAGCCCAACTAGCGATCACCAC
TGGTGTATTCCGTTTACGGTAAAAGGAAATCCAAAACCTGCACTCCAATG
GTTTTATAATGGAGCCATCTTGAATGAAAGCAAATATATCTGTACTAAAA
TCCATGTGACGAATCACACCGAGTATCACGGGTGTCTTCAATTGGATAAT
CCAACCCATATGAATAATGGTGATTATACTTTGATAGCGAAGAACGAATA
CGGCAAAGACGAAAAGCAAATATCCGCACATTTCATGGGTTGGCCTGGCA
TCGACGACGGTGCGAACCCGAACTACCCAGATGTTATTTACGAGGATTAT
GGGACTGCGGCAAACGACATTGGCGACACCACAAACCGAAGCAACGAGAT
ACCAAGTACTGACGTCACTGACAAAACGGGTCGAGAGCATTTGTCTGTTT
ACGCCGTTGTTGTTATCGCCTCAGTTGTCGGATTTTGCCTGTTGGTCATG
CTTTTCCTCCTGAAGCTCGCGCGACATTCCAAGTTTGGCATGAAGGGG C
CAGCAAGTGTTATATCCAATGATGATGATAGCGCTTCTCCATTGCACCAC
ATAAGTAACGGCTCAAACACGCCGTCATCTAGTGAAGGTGGACCAGACGC
GGTCATTATAGGGATGACTAAAATTCCCGTAATCGAAAACCCTCAGTACT
TCGGCATAACCAACAGTCAGCTTAAACCCGATACTTTCGTGCAGCACATC
AAAAGGCACAACATAGTCCTCAAGCGCGAACTCGGGGAGGGAGCCTTCGG
AAAGGTCTTTCTTGCTGAGTGCTATAATTTGTGTCCTGAGCAGGATAAAA
TTCTTGTGGCTGTAAAAACTCTCAAAGATGCTTCCGACAACGCACGGAAG
GATTTTCATCGGGAGGCCGAACTGTTGACGAATTTGCAGCACGAGCATAT
AGTAAAGTTCTACGGGGTATGTGTTGAGGGGGACCCGTTGATTATGGTCT
TCGAGTATATGAAGCACGGGGACCTGAACAAATTTTTGCGCGCCCATGGG
CCTGATGCCGTCCTTATGGCAGAAGGGAACCCTCCAACAGAACTCACCCA
GAGTCAGATGTTGCACATAGCGCAACAGATCGCGGCCGGCATGGTTTACC
TGGCCAGTCAACACTTCGTGCATAGAGATCTTGCCACTCGCAACTGTTTG
GTCGGGGAGAACCTTCTGGTTAAGATTGGTGACTTTGGTATGTCACGAGA
TGTGTATTCCACTGACTATTACAGAGTTGGGGGTCATACAATGCTTCCTA
TTCGGTGGATGCCCCCCGAATCCATCATGTACAGAAAGTTCACGACAGAG
AGTGATGTT TGG AGT CTCGGCGTGGTGCTCTGGGAAATTTTCACATA
CGGAAAGCAGCCGTGGTATCAACTTAGCAACAATGAGGTGATAGAGTGTA
TTACACAGGGTCGGGTGTTGCAGCGCCCTCGAACGTGCCCACAAGAAGTA
TATGAACTTATGCTCGGGTGCTGGCAAAGAGAACCACATATGAGAAAAAA
TATCAAGGGGATACATACATTGCTTCAGAACTTGGCCAAGGCATCACCCG
TCTACCTCGATATACTGGGCAGCGGAGCTACTAACTTCAGCCTGCTGAAG
CAGGCTGGAGACGTGGAGGAGAACCCTGGACCTATGAGAATCCTTCTTCT
TACTATGGTTATTTCATACTTCGGTTGCATGAAGGCGCACTCCGACCCTG
CCCGCCGTGGGGAGCTGAGCGTGTGTGACAGTATTAGCGAGTGGGTCACA
GCGGCAGATAAAAAGACTGCAGTGGACATGTCTGGCGGGACGGTCACAGT
CCTAGAGAAAGTCCCGGTATCCAAAGGCCAACTGAAGCAGTATTTCTACG
AGACCAAGTGTAATCCCATGGGTTACACCAAGGAAGGCTGCAGGGGCATA
GACAAAAGGCACTGGAACTCGCAATGCCGAACTACCCAATCGTATGTTCG
GGCCCTTACTATGGATAGCAAAAAGAGAATTGGCTGGCGATTCATAAGGA
TAGACACTTCCTGTGTATGTACACTGACCATTAAAAGGGGAAGATAG

Hence, in a most preferred embodiment, the construct comprises a nucleotide sequence substantially as set out in SEQ ID No: 107 or 108, or a fragment or variant thereof.

The inventors have created a series of recombinant expression vectors comprising the construct of the invention.

Thus, according to a second aspect, there is provided a recombinant vector comprising the genetic construct according to the first aspect, for use in the treatment, prevention or amelioration of a neurodegenerative disorder or stroke.

The constructs and expression vectors described herein can be used to promote nerve regeneration and survival. In some embodiments, the recombinant vector is for the treatment, prevention or amelioration of Alzheimer's disease, Huntington's disease, Parkinson's disease, motor neurone disease, or stroke. The recombination vectors described herein may be for any treatment or use as described herein.

The recombinant vector may be a recombinant AAV (rAAV) vector. The rAAV may be a naturally occurring vector or a vector with a hybrid AAV serotype. The rAAV may be AAV-1, AAV-2, AAV-3A, AAV-3B, AAV-4, AAV-5, AAV-6, AAV-7, AAV-8, AAV-9, AAV-10, and AAV-11. Preferably, the rAAV is rAAV serotype-2.

Advantageously, recombinant AAV2 evokes a minimal immune response in host organisms and mediates long-term transgene expression that can persist for at least one year after vector administration.

The term “recombinant AAV (rAAV) vector” as used herein means a recombinant AAV-derived nucleic acid containing at least one terminal repeat sequence.

Preferred embodiments of the vector are shown in FIGS. 2-5.

According to a third aspect, there is provided a method of treating, preventing or ameliorating a neurodegenerative disorder or stroke in a subject, or for promoting nerve regeneration and/or survival in a subject, the method comprising administering, to a subject in need of such treatment, a therapeutically effective amount of the genetic construct according to the first aspect, or the recombinant vector according to the second aspect.

In some embodiments, the method may be for the treatment, prevention, or amelioration of Alzheimer's disease, Parkinson's disease, motor neurone disease, Huntington's disease, or any other neurodegenerative disclosed herein.

Preferably, the genetic construct or the recombinant vector according to invention are used in a gene therapy technique. The agonist encoded by the construct or vector activate the TrkB also encoded by the construct/vector to thereby promote survival of neuronal cells.

In another embodiment, the constructs and vectors may be used to promote nerve regeneration and/or survival.

It will be appreciated that the genetic construct according to the first aspect, or the recombinant vector according to the second aspect may be used in a medicament, which may be used as a monotherapy (i.e. use of the genetic construct according to the first aspect or the vector according to the second aspect of the invention), for treating, ameliorating, or preventing a neurodegenerative disorder or stroke, or for promoting nerve regeneration and/or survival. Alternatively, the genetic construct or the recombinant vector according to the invention may be used as an adjunct to, or in combination with, known therapies for treating, ameliorating, or preventing a neurodegenerative disorder or stroke, or for promoting nerve regeneration and/or survival.

The genetic construct according or the recombinant vector according to the invention may be combined in compositions having a number of different forms depending, in particular, on the manner in which the composition is to be used. Thus, for example, the composition may be in the form of a powder, tablet, capsule, liquid, ointment, cream, gel, hydrogel, aerosol, spray, micellar solution, transdermal patch, liposome suspension or any other suitable form that may be administered to a person or animal in need of treatment. It will be appreciated that the vehicle of medicaments according to the invention should be one which is well-tolerated by the subject to whom it is given.

The genetic construct or the recombinant vector according to the invention may also be incorporated within a slow- or delayed-release device. Such devices may, for example, be inserted on or under the skin, and the medicament may be released over weeks or even months. The device may be located at least adjacent the treatment site. Such devices may be particularly advantageous when long-term treatment with the genetic construct or the recombinant vector is required and which would normally require frequent administration (e.g. at least daily injection).

In a preferred embodiment, medicaments according to the invention may be administered to a subject by injection into the blood stream, a nerve or directly into a site requiring treatment. For example, the medicament is configured to cross the blood-brain-barrier. Injections may be intravenous (bolus or infusion) or subcutaneous (bolus or infusion), or intradermal (bolus or infusion).

It will be appreciated that the amount of the genetic construct or the recombinant vector that is required is determined by its biological activity and bioavailability, which in turn depends on the mode of administration, the physiochemical properties of the genetic construct or the recombinant vector and whether it is being used as a monotherapy or in a combined therapy. The frequency of administration will also be influenced by the half-life of the cyclic polypeptide within the subject being treated.

Optimal dosages to be administered may be determined by those skilled in the art, and will vary with the particular genetic construct or the recombinant vector in use, the strength of the pharmaceutical composition, the mode of administration, and the advancement or stage of the disorder. Additional factors depending on the particular subject being treated will result in a need to adjust dosages, including subject age, weight, gender, diet, and time of administration.

Generally, a daily dose of between 0.001 μg/kg of body weight and 10 mg/kg of body weight, or between 0.01 μg/kg of body weight and 1 mg/kg of body weight, of the construct or vector according to the invention may be used for treating, ameliorating, or preventing a neurodegenerative disorder, Alzheimer's disease, Parkinson's disease, Huntington's disease, motor neurone disease, or stroke, depending upon the genetic construct or recombinant vector used.

The genetic construct or the recombinant vector may be administered before, during or after onset of the disorder. Daily doses may be given as a single administration (e.g. a single daily injection or inhalation of a nasal spray). Alternatively, the genetic construct or the recombinant vector may require administration twice or more times during a day. As an example, the genetic construct or the recombinant vector may be administered as two (or more depending upon the severity of the disorder being treated) daily doses of between 0.07 μg and 700 mg (i.e. assuming a body weight of 70 kg). A patient receiving treatment may take a first dose upon waking and then a second dose in the evening (if on a two dose regime) or at 3- or 4-hourly intervals thereafter. Alternatively, a slow release device may be used to provide optimal doses of the genetic construct or the recombinant vector according to the invention to a patient without the need to administer repeated doses.

Known procedures, such as those conventionally employed by the pharmaceutical industry (e.g. in vivo experimentation, clinical trials, etc.), may be used to form specific formulations of the genetic construct or the recombinant vector according to the invention and precise therapeutic regimes (such as daily doses of the agents and the frequency of administration). The inventors believe that they are the first to suggest a genetic construct encoding promoter operably linked to coding sequences of a TrkB receptor and a TrkB receptor agonist.

According to a fourth aspect, there is provided a pharmaceutical composition comprising the genetic construct according to the first aspect, or the recombinant vector according to the second aspect, and a pharmaceutically acceptable vehicle.

According to a fifth aspect, there is provided a method of preparing the pharmaceutical composition according to the fifth aspect, the method comprising contacting the genetic construct according to the first aspect, or the recombinant vector according to the second aspect, with a pharmaceutically acceptable vehicle.

A “subject” may be a vertebrate, mammal, or domestic animal. Hence, compositions and medicaments according to the invention may be used to treat any mammal, for example livestock (e.g. a horse), pets, or may be used in other veterinary applications. Most preferably, however, the subject is a human being.

A “therapeutically effective amount” of the genetic construct, the recombinant vector or the pharmaceutical composition is any amount which, when administered to a subject, is the amount of the aforementioned that is needed to treat a neurodegenerative disorder, Alzheimer's disease, Parkinson's disease, Huntington's disease, motor neurone disease, stroke, or produce the desired effect, such as promoting nerve regeneration and/or survival.

For example, the therapeutically effective amount of the genetic construct, the recombinant vector or the pharmaceutical composition used may be from about 0.01 mg to about 800 mg, and preferably from about 0.01 mg to about 500 mg. It is preferred that the amount of the genetic construct, the recombinant vector or the pharmaceutical composition is an amount from about 0.1 mg to about 250 mg, and most preferably from about 0.1 mg to about 20 mg.

A “pharmaceutically acceptable vehicle” as referred to herein, is any known compound or combination of known compounds that are known to those skilled in the art to be useful in formulating pharmaceutical compositions.

In one embodiment, the pharmaceutically acceptable vehicle may be a solid, and the composition may be in the form of a powder or tablet. A solid pharmaceutically acceptable vehicle may include one or more substances which may also act as flavouring agents, lubricants, solubilisers, suspending agents, dyes, fillers, glidants, compression aids, inert binders, sweeteners, preservatives, dyes, coatings, or tablet-disintegrating agents. The vehicle may also be an encapsulating material. In powders, the vehicle is a finely divided solid that is in admixture with the finely divided active agents according to the invention. In tablets, the active agent (e.g. the genetic construct or recombinant vector according to the invention) may be mixed with a vehicle having the necessary compression properties in suitable proportions and compacted in the shape and size desired. The powders and tablets preferably contain up to 99% of the active agents. Suitable solid vehicles include, for example calcium phosphate, magnesium stearate, talc, sugars, lactose, dextrin, starch, gelatin, cellulose, polyvinylpyrrolidine, low melting waxes and ion exchange resins. In another embodiment, the pharmaceutical vehicle may be a gel and the composition may be in the form of a cream or the like.

However, the pharmaceutical vehicle may be a liquid, and the pharmaceutical composition is in the form of a solution. Liquid vehicles are used in preparing solutions, suspensions, emulsions, syrups, elixirs and pressurized compositions. The genetic construct or the recombinant vector according to the invention may be dissolved or suspended in a pharmaceutically acceptable liquid vehicle such as water, an organic solvent, a mixture of both or pharmaceutically acceptable oils or fats. The liquid vehicle can contain other suitable pharmaceutical additives such as solubilisers, emulsifiers, buffers, preservatives, sweeteners, flavouring agents, suspending agents, thickening agents, colours, viscosity regulators, stabilizers or osmo-regulators. Suitable examples of liquid vehicles for oral and parenteral administration include water (partially containing additives as above, e.g. cellulose derivatives, preferably sodium carboxymethyl cellulose solution), alcohols (including monohydric alcohols and polyhydric alcohols, e.g. glycols) and their derivatives, and oils (e.g. fractionated coconut oil and arachis oil). For parenteral administration, the vehicle can also be an oily ester such as ethyl oleate and isopropyl myristate. Sterile liquid vehicles are useful in sterile liquid form compositions for parenteral administration. The liquid vehicle for pressurized compositions can be a halogenated hydrocarbon or other pharmaceutically acceptable propellant.

Liquid pharmaceutical compositions, which are sterile solutions or suspensions, can be utilized by, for example, intramuscular, intrathecal, epidural, intraperitoneal, intravenous and particularly subcutaneous injection. The genetic construct or the recombinant vector may be prepared as a sterile solid composition that may be dissolved or suspended at the time of administration using sterile water, saline, or other appropriate sterile injectable medium.

The genetic construct, the recombinant vector and the pharmaceutical composition of the invention may be administered orally in the form of a sterile solution or suspension containing other solutes or suspending agents (for example, enough saline or glucose to make the solution isotonic), bile salts, acacia, gelatin, sorbitan monoleate, polysorbate 80 (oleate esters of sorbitol and its anhydrides copolymerized with ethylene oxide) and the like. The genetic construct, the recombinant vector or the pharmaceutical composition according to the invention can also be administered orally either in liquid or solid composition form. Compositions suitable for oral administration include solid forms, such as pills, capsules, granules, tablets, and powders, and liquid forms, such as solutions, syrups, elixirs, and suspensions. Forms useful for parenteral administration include sterile solutions, emulsions, and suspensions.

According to a further aspect, there is provided the genetic construct according to the first aspect, or the recombinant vector according to the second aspect, for use in treating, preventing or ameliorating an optic nerve disorder or a cochlear disorder, or for promoting nerve regeneration and/or survival; wherein the second coding sequence comprises the mature form of a trophic factor from the neurotrophin family. The second coding sequence may comprise a signal peptide. The construct or vector may be such that the agonist lacks the pro-sequence but has a signal peptide. The signal peptide may be attached to the N-terminus and may boost secretion, expression, or folding of the agonist. The second coding sequence may comprise any of: mature nerve growth factor (NGF), mature neurotrophin-3 (NT-3), mature neurotrophin-5 (NT-5), or fragments or variants thereof.

It will be appreciated that the invention extends to any nucleic acid or peptide or variant, derivative or analogue thereof, which comprises substantially the amino acid or nucleic acid sequences of any of the sequences referred to herein, including variants or fragments thereof. The terms “substantially the amino acid/nucleotide/peptide sequence”, “variant” and “fragment”, can be a sequence that has at least 40% sequence identity with the amino acid/nucleotide/peptide sequences of any one of the sequences referred to herein, for example 40% identity with the sequence identified as SEQ ID No:1-108, and so on.

Amino acid/polynucleotide/polypeptide sequences with a sequence identity which is greater than 65%, more preferably greater than 70%, even more preferably greater than 75%, and still more preferably greater than 80% sequence identity to any of the sequences referred to are also envisaged. Preferably, the amino acid/polynucleotide/polypeptide sequence has at least 85% identity with any of the sequences referred to, more preferably at least 90% identity, even more preferably at least 92% identity, even more preferably at least 95% identity, even more preferably at least 97% identity, even more preferably at least 98% identity and, most preferably at least 99% identity with any of the sequences referred to herein.

The skilled technician will appreciate how to calculate the percentage identity between two amino acid/polynucleotide/polypeptide sequences. In order to calculate the percentage identity between two amino acid/polynucleotide/polypeptide sequences, an alignment of the two sequences must first be prepared, followed by calculation of the sequence identity value. The percentage identity for two sequences may take different values depending on:—(i) the method used to align the sequences, for example, ClustalW, BLAST, FASTA, Smith-Waterman (implemented in different programs), or structural alignment from 3D comparison; and (ii) the parameters used by the alignment method, for example, local vs global alignment, the pair-score matrix used (e.g. BLOSUM62, PAM250, Gonnet etc.), and gap-penalty, e.g. functional form and constants.

Having made the alignment, there are many different ways of calculating percentage identity between the two sequences. For example, one may divide the number of identities by: (i) the length of shortest sequence; (ii) the length of alignment; (iii) the mean length of sequence; (iv) the number of non-gap positions; or (v) the number of equivalenced positions excluding overhangs. Furthermore, it will be appreciated that percentage identity is also strongly length dependent. Therefore, the shorter a pair of sequences is, the higher the sequence identity one may expect to occur by chance.

Hence, it will be appreciated that the accurate alignment of protein or DNA sequences is a complex process. The popular multiple alignment program ClustalW (Thompson et al., 1994, Nucleic Acids Research, 22, 4673-4680; Thompson et al., 1997, Nucleic Acids Research, 24, 4876-4882) is a preferred way for generating multiple alignments of proteins or DNA in accordance with the invention. Suitable parameters for ClustalW may be as follows: For DNA alignments: Gap Open Penalty=15.0, Gap Extension Penalty=6.66, and Matrix=Identity. For protein alignments: Gap Open Penalty=10.0, Gap Extension Penalty=0.2, and Matrix=Gonnet. For DNA and Protein alignments: ENDGAP=−1, and GAPDIST=4. Those skilled in the art will be aware that it may be necessary to vary these and other parameters for optimal sequence alignment.

Preferably, calculation of percentage identities between two amino acid/polynucleotide/polypeptide sequences may then be calculated from such an alignment as (N/T)*100, where N is the number of positions at which the sequences share an identical residue, and T is the total number of positions compared including gaps but excluding overhangs. Hence, a most preferred method for calculating percentage identity between two sequences comprises (i) preparing a sequence alignment using the ClustalW program using a suitable set of parameters, for example, as set out above; and (ii) inserting the values of N and T into the following formula:—Sequence Identity=(N/T)*100.

Alternative methods for identifying similar sequences will be known to those skilled in the art. For example, a substantially similar nucleotide sequence will be encoded by a sequence which hybridizes to DNA sequences or their complements under stringent conditions. By stringent conditions, we mean the nucleotide hybridises to filter-bound DNA or RNA in 3× sodium chloride/sodium citrate (SSC) at approximately 45° C. followed by at least one wash in 0.2×SSC/0.1% SDS at approximately 20-65° C. Alternatively, a substantially similar polypeptide may differ by at least 1, but less than 5, 10, 20, 50 or 100 amino acids from the sequences shown in, for example, SEQ ID Nos: 3 and 5.

Due to the degeneracy of the genetic code, it is clear that any nucleic acid sequence described herein could be varied or changed without substantially affecting the sequence of the protein encoded thereby, to provide a functional variant thereof. Suitable nucleotide variants are those having a sequence altered by the substitution of different codons that encode the same amino acid within the sequence, thus producing a silent change. Other suitable variants are those having homologous nucleotide sequences but comprising all, or portions of, sequence, which are altered by the substitution of different codons that encode an amino acid with a side chain of similar biophysical properties to the amino acid it substitutes, to produce a conservative change. For example small non-polar, hydrophobic amino acids include glycine, alanine, leucine, isoleucine, valine, proline, and methionine. Large non-polar, hydrophobic amino acids include phenylalanine, tryptophan and tyrosine. The polar neutral amino acids include serine, threonine, cysteine, asparagine and glutamine. The positively charged (basic) amino acids include lysine, arginine and histidine. The negatively charged (acidic) amino acids include aspartic acid and glutamic acid. It will therefore be appreciated which amino acids may be replaced with an amino acid having similar biophysical properties, and the skilled technician will know the nucleotide sequences encoding these amino acids.

All of the features described herein (including any accompanying claims, abstract and drawings), and/or all of the steps of any method or process so disclosed, may be combined with any of the above aspects in any combination, except combinations where at least some of such features and/or steps are mutually exclusive.

For a better understanding of the invention, and to show how embodiments of the same may be carried into effect, reference will now be made, by way of example, to the accompanying Figure, in which:—

FIG. 1 is schematic of one embodiment of a genetic construct according to the invention;

FIG. 2 is a schematic drawing of a first embodiment of a recombinant vector according to the invention known as “Plasmid QTA001PA” containing canonical signal sequence (blue) plus proBDNF (red) and mBDNF (black). It also includes an -IRES-GFP- sequence (cyan and purple);

FIG. 3 is a schematic drawing of a second embodiment of the recombinant vector according to the invention known as “Plasmid QTA002P” with no proBDNF (but produces only mBDNF) and same signal sequence (blue) as QTA001PA. It also includes an -IRES-GFP- sequence (cyan and purple);

FIG. 4 is a schematic drawing of a third embodiment of the recombinant vector according to the invention known as of “Plasmid QTA003P” with no proBDNF (but produces only mBDNF) and IL-2 signal sequence (blue). It also includes an -IRES-GFP- sequence (cyan and purple);

FIG. 5 is a schematic drawing of a fourth embodiment of a recombinant vector according to the invention known as “Plasmid QTA004P” with no proBDNF (but produces only mBDNF) and a novel signal sequence (blue). It also includes an -IRES-GFP- sequence (cyan and purple);

FIG. 6 shows nucleotide and amino acid sequences for different embodiments of signal peptide used in the construct of the invention. The second residue is threonine (t) which can be replaced by one or more basic residue, such as lysine (K) or arginine (R). The next stretch of residues including isoleucine (I), leucine (L), phenylalanine (F) and Leucine (L) can be replaced by one or more hydrophobic residues;

FIG. 7 shows release of BDNF from HEK293 cells using a specific ELISA at 24 hours following transduction of a plasmid (4 μg DNA/well) containing genes coding for mBDNF with differing signal peptide sequences and without the coding sequence for the extended proBDNF component (Data shown as mean±SEM for n=4);

FIG. 8 shows Western blotting results of cellular concentrations of BDNF-immunoreactive material (arbitrary units) in HEK293 cell lysates 24 hours after plasmid transduction (Data shown as mean±SEM for n=4);

FIG. 9 shows BDNF-immunoreactivity in Western blots of cell lysates showing two molecular weight bands (32 kDa and 14 kDa) when cells were transduced with QTA001PA, versus only a single 14 kDa band with QTA002P, QTA003P and QTA004P transduction;

FIG. 10 shows proBDNF concentrations in the HEK293 incubation medium as measured using a specific ELISA 24 hours after plasmid transduction using a selective proBDNF ELISA (Data shown as mean±SEM for n=4);

FIG. 11 shows BDNF expression in HEK293 cell lysate by plasmids QTA002P (endogenous canonical signal peptide sequence), and QTA009P to QTA013P. Data is shown as mean+S.E.M. ** P<0.01 as compared to QTA002P;

FIG. 12 shows BDNF expression in HEK293 cell incubation medium by plasmids QTA002P (endogenous canonical signal peptide sequence), and QTA009P to QTA013P. Data is shown as mean+S.E.M. ** P<0.01 as compared to QTA002P;

FIG. 13 shows Western Blots from HEK293 cells 24 hours after they were transduced with plasmids QTA015P (expressing BDNF and eGFP separated by an IRES spacer), QTA021P (expressing BDNF followed by eGFP separated by a functional viral-2A peptide sequence), QTA022P (expressing BDNF followed by eGFP separated by a non-functional viral-2A peptide sequence) and QTA023P (expressing eGFP followed by coding for BDNF separated by a functional viral-2A peptide sequence). Data shown as BDNF-immunoreactivity (A), eGFP-immunoreactivity (B) and the amount of BDNF released from the HEK293 cells into the incubation medium (C). Data is shown as mean+S.E.M of the density in the bands;

FIG. 14A shows Western blot of HEK293 cell homogenates 48 hours after transfection with the QTA020V vector and showing efficient processing of the large precursor coding region which includes the TrkB receptor and BDNF separated by the viral-2A peptide sequence. FIGS. 14B and 14C show that the transgene proteins produced after vial-2A peptide cleavage have been transported to the correct intracellular compartments in HEK293 cells after processing (TrkB receptors to the cell surface and BDNF to storage vesicles prior to release);

FIG. 15A shows TrkB receptor expression and FIG. 15B shows BDNF expression in mouse retinal homogenate for the rAAV2 vector, QTA020V. Data is shown as mean+S.E.M of the density in the Western blot of mouse retina homogenates. ** P<0.01 as compared to naïve (un-injected animals);

FIG. 16 shows expression of TrkB (A) and BDNF (B) transgenes in mouse retinal ganglion cell layer as shown by immunocytochemistry following injection of QTA020V, a rAAV2 vector containing the coding for the TrkB receptor and BDNF, separated by the viral-2A peptide sequence;

FIG. 17 shows retinal ganglion cell (RGC) survival following optic nerve crush (ONC) in the mouse versus control animals treated with rAAV2-CAG-eGFP vector. Data shown as mean+S.E.M. for average numbers of retinal ganglion cells throughout the retina per animal as counted by Brn3A-positive cells in retinal flat-mounts. ***P<0.001, *P<0.05 as compared to controls;

FIG. 18 shows expression of BDNF (FIG. 18A) and TrkB (FIG. 18B) transgenes in undifferentiated human SH-SY5Y neuroblastoma cell homogenates by Western blotting following transfection with rAAV2 viral vectors which express no transgenes (Null virus), BDNF only (QTA027V), TrkB only (QTA025V) and both BDNF and TrkB (QTA020V). FIG. 18C shows the level of activated phosphorylated TrkB receptors in the SH-SY5Y cells in Western blots following transfection with the viral vectors Null, QTA020V, QTA025V or QTA027V. Only QTA020V vector which expresses both BDNF and TrkB was found to significantly increase the activation of TrkB receptors, as compared to untransfected cells (**P<0.01; ANOVA followed by Bonferroni modified t-tests for multiple comparisons). Data shown as mean+S.E.M. for n=4 experiments;

FIG. 19 shows the level of apoptotic cell death of undifferentiated SH-SY5Y cells in culture following exposure to oxidative stress produced by addition of hydrogen peroxide (H₂O₂ at either 0.1 mM or 1.0 mM) by TUNEL staining. Cells transfected with the rAAV2 vector QTA020V, which expresses both BDNF and TrkB receptors, prior to addition of the hydrogen peroxide were found to be significantly protected against apoptosis versus untreated cells (**P<0.01; ANOVA followed by Bonferroni modified t-tests for multiple comparisons). Data shown as mean+S.E.M. for n=6-10; and

FIG. 20 shows representative immunocytochemical images of optic nerves obtained from P301S mutant human Tau transgenic mice and stained with antibodies which recognise phosphorylated Tau at positions serine 396/serine 404 (PHF-1) or serine 202/serine 205 (AT8). Mice were injected intravitreally with the rAAV2 vector QTA020V (which expresses both mBDNF and TrkB receptors) at 3 months old and terminated three weeks later prior to removal of optic nerves for immunocytochemistry.

### EXAMPLES

The following Examples demonstrate the use of embodiments of the present invention to promote nerve regeneration and/or survival. The teaching derivable from the uses, methods, and treatments disclosed by the Examples is also applicable to the treatment of the neurodegenerative disorders and stroke, as disclosed herein.

Methods and Materials

Molecular Cloning and Plasmid Constructs

Codon optimisation of DNA sequences was performed using the on-line tool (http: www.idtdna.corn/CodonOpt) and DNA blocks were synthesised by Integrated DNA technologies, Inc. (IDT; 9180 N. McCormick Boulevard, Skokie, Ill. 60076-2920, USA) or GenScript (860 Centennial Ave, Piscataway, N.J. 08854, USA). Cloning to make the master plasmid QTA001PA and subsequent plasmids were performed using standard molecular biology and cloning techniques.

Plasmid Scale Up and Purification

DNA Plasmids were scaled up in SURE competent cells (Agilent Technologies; cat. #200238) overnight to provide 2.29 μg/μl plasmid following maxi-prep purification. The remaining plasmids were scaled up to 500 μg scale and transduction quality with minimal endotoxin presence.

HEK293 Culture and Cell Transduction with Plasmid DNA

HEK293 cells (400,000 cells) were cultured in poly-L-lysine (10 ug/mL, Sigma-Aldrich; cat. #P1274) coated 6 well plates in 1.5 mL Dulbecco's minimum essential medium (DMEM) containing 10% foetal bovine serum (FBS), 1% penicillin and 1% streptomycin (1% Pen/Strep) until 80% confluent. The medium was then exchanged for 2 mL DMEM (no additives). Two to three hours later, an additional 0.5 ml transfection medium containing 4 g plasmid DNA plus 10 μL lipofectamine (4 μL/mL; Thermo Fisher Scientific; cat. #12566014) was added to each well resulting in an overall volume of 2.5 ml throughout the transfection period and for supernatant collection.

SH-SY5Y Culture and Cell Transfection with rAAV2 Viral Vectors

35 SH-SY5Y cells were cultured in 6 well plates (300,000 cells), 96 well plates (10,000 cells) or on 13 mm glass coverslips (100,000 cells) coated with poly-L-lysine (10 μg/mL, Sigma product #P1274). Dulbecco's minimum essential medium (DMEM) containing 10% foetal bovine serum (FBS), 1% penicillin and 1% streptomycin (1% Pen/Strep) was used to culture cells to 80% confluent at 37° C. prior to exchange to DMEM with no additives prior to transfection. DMEM volumes used were 6 well plates (2 mL), 96 well plate (100 μL), coverslips (500 μL). Vectors, diluted in PBS, were added directly to the culture medium at a final concentration of 1.0×10¹⁰(VP)/mL and incubated for 48 hours at 37° C.

Hydrogen Peroxide-Induced SH-SY5Y Cell Death and TUNEL Staining

48 hours after SH-SY5Y cell transfection, medium was exchanged for fresh DMEM (no additives). Hydrogen peroxide (H₂O₂) (Thermo Fisher Scientific; product #BP2633500, lot #1378087) was diluted in filtered water (to a concentration of 0.1 or 1.0 mM) and added at an equal volume to wells or plates for an additional 24 hours. Filtered water served as a vehicle control. Coverslips were washed twice in PBS and fixed for 30 min in 4% paraformaldehyde in 1M phosphate buffered saline (PBS) at room temperature. After three more washes in PBS, cells were blocked and permeabilized by incubation in 5% normal goat serum (NGS), 3% bovine serum albumin (BSA) and 0.3% Triton X-100 in PBS for 60 minutes at room temperature. Cells were then incubated overnight at 4° C. with commercial rabbit polyclonal antibodies for TrkB (Abcam; product #ab33655, lot #GR232306-1 diluted 1:500), rabbit polyclonal anti-BDNF antibodies (Santa Cruz Biotechnology Inc; product #sc-546; lot #C0915 at 1:300 dilution) or p-Tyr⁵¹⁵-TrkB (Abcam product #ab109684 lot #GR92849-4 1:750) diluted in blocking solution. Staining was revealed using secondary anti-rabbit antibodies conjugated to alexa fluor 488 (Life Technologies; product #A11034 at 1:1000) for 2 hours at room temperature. For TUNEL staining (Promega; product #G3250; lot #0000215719), cells were washed three times in PBS and immersed in TUNEL equilibration buffer for 10 minutes. The TUNEL reaction mixture was made per the manufacturers protocol and 100 μL/coverslip added to cells for 1 hour at 37° C. The reaction was stopped by incubating in 1× standard citrate solution (SCS) for 15 minutes. Cell nuclei were counterstained with 1 μg/mL DAPI (Thermo Scientific; product #D1306 at 1:8000). Cells were further washed three times before being mounted with fluorSave™ reagent (Calbiochem/EMD Chemicals Inc., Gibbstown, N.J., USA) prior to imaging. Imaging was carried out using a 20× objective and a Leica DM6000 epifluorescence microscope (Leica Microsystems, Wetzlar, Germany).

BDNF Measurement by ELISA

The amount of BDNF secreted from HEK293 cells was measured in cell culture medium 24 hours after transfection. Medium was centrifuged, to remove debris, and measured using a commercial Human BDNF ELISA kit (Sigma-Aldrich, product #RAB0026). BDNF concentration was determined by comparing samples to freshly made BDNF standards.

Western Blotting for BDNF and TrkB Receptors

The amount of BDNF and TrkB-immunoreactivity within the HEK293 cells was measured by removing the DMEM incubation medium, washing the cells in cold phosphate buffered saline and the addition of 350 μL freshly prepared lysis buffer to the wells (10 ml Lysis-M reagent+1 tablet of complete Mini Protease Inhibitor Cocktail, Roche; cat. #04719964001, +100 μl Halt phosphatase inhibitor cocktail (100×), Thermo Scientific; cat. #78428). After cell homogenisation, the protein suspension was quantified using the BCA assay (Pierce BCA protein assay kit, Thermo Scientific; cat. #23227). Between 6 μg and 15 μg HEK293 cell lysate protein/lane were run down a Bis-Tris gel (12% NuPAGE Novex; cat. #NP0342BOX, Thermo Scientific) and examined by Western blotting using the primary rabbit polyclonal anti-BDNF antibodies (Santa Cruz Biotechnology Inc; product #sc-546; at 1:500 dilution), rabbit polyclonal anti-TrkB antibodies (Abcam; cat. #ab33655, used at 1:2000 dilution) or eGFP antibodies (Abcam product #ab-290 used at 1:500) which were incubated overnight. Primary antibodies were visualised with HRP conjugated anti-rabbit antibodies (Vector Laboratories; cat. #PI-1000, at 1:8000) and signal detection using ECL Prime (Amersham, GE Healthcare, UK) and an Alliance Western blot imaging system (UVItec Ltd, Cambridge, UK). For Western blots of mouse retina, eyes from vector-treated animals were homogenized in 5000 L freshly prepared lysis buffer (10 ml Lysis-M reagent+1 tablet of cOmplete Mini Protease Inhibitor Cocktail, Roche product #04719964001+100 μl Halt phosphatase inhibitor cocktail (100×), Thermo Scientific product #78428). Tissue was disrupted for 1 minute (Qiagen, TissueRuptor product #9001273) and then kept on ice for an additional 15 minutes. The protein was then analysed by Western blotting as described above.

Immunocytochemistry

HEK293 cells (70,000) were seeded on 13 mm, poly-L-lysine coated coverslips within 4 well plates and incubated in DMEM containing 10% FBS and 1% Pen/Strep in 0.5 ml medium. Once the cells had grown to 80% confluence, the medium was exchanged for 0.4 ml DMEM (no additives) for 2-3 hours then an additional 0.1 mL transfection medium (0.8 μg plasmid DNA+2 μl lipofectamine) was added so that the final volume reached 0.5 ml. Coverslips were washed twice in PBS and fixed for 30 min in 4% paraformaldehyde in 1M phosphate buffered saline (PBS) at room temperature. After three more washes in PBS, cells were blocked and permeabilized by incubation in 5% normal goat serum (NGS), 3% bovine serum albumin (BSA) and 0.3% Triton X-100 in PBS for 60 minutes at room temperature. Cells were then incubated overnight at 4° C. with commercial rabbit polyclonal antibodies for BDNF (Santa Cruz Biotechnology Inc; product #sc-546; at 1:300 dilution) or TrkB (Abcam product #ab33655, diluted 1:500) diluted in blocking solution. Staining was revealed using secondary anti-rabbit antibodies conjugated to alexa fluor 647 (Invitrogen, product #A21248 at 1:1000) for 2 hours at room temperature. Cell nuclei were also counterstained with 1 g/ml DAPI (Thermo Scientific, product #D1306 at 1:8000). Cells were further washed three times before being mounted with fluorSave™ reagent (Calbiochem/EMD Chemicals Inc., Gibbstown, N.J., USA) prior to imaging. Imaging was carried out using a 20× objective and a Leica DM6000 epifluorescence microscope (Leica Microsystems, Wetzlar, Germany) or a Leica SP5 confocal microscope (Leica Microsystems, Wetzlar, Germany) equipped with a 63× oil objective using a 3× digital zoom and 0.5-0.8 sequential scanning z-step interval.

For immunocytochemistry of retinal structures and optic nerves from control or vector treated animals (at between 3 or 4 weeks following injection), carefully dissected eyes were fixed in 4% paraformaldehyde/0.1% PBS (pH 7.4) overnight and dehydrated in 30% sucrose/0.1% PBS at 4° C. (24 hours). Eyes were then embedded in silicon moulds containing optimal cutting temperature compound (OCT) (Sakura Finetek, Zoeterwoude, Netherlands) and frozen on dry ice. Thirteen m sections through the dorsal-ventral/superior-inferior axis of the retina or longitudinal sections through the optic nerve of P301S mice were collected onto superfrost plus slides (VWR product #631-0108), using a Bright OTF 5000 cryostat (Bright Instruments, Huntingdon, UK). Slides were washed three times in PBS, and permeabilized in 5% normal goat serum (NGS), 3% bovine serum albumin (BSA) and 0.3% Triton X-100 in PBS for 60 minutes at room temperature. Slides were then incubated overnight at 4° C. with commercial rabbit polyclonal antibodies for BDNF (Santa Cruz Biotechnology Inc; product #sc-546 1:300), TrkB (Abcam; product #ab33655 1:500), Tau Ser^{396/404} (PHF-1; generated in Cambridge 1:500) or Tau Ser^{202/205} (AT8; Invitrogen product #MN1020 1:500) diluted in blocking solution. Staining was revealed using secondary anti-rabbit antibodies conjugated to alexa fluor 647 (Invitrogen, product #A21248 at 1:1000) for 2 hours at room temperature. Retinal cell nuclei were also counterstained with 1 μg/mL DAPI (Thermo Scientific, product #D1306 at 1:8000). Slides were further washed three times before being mounted with fluorSave™ reagent (Calbiochem/EMD Chemicals Inc., Gibbstown, N.J., USA) prior to imaging. Imaging was carried out using a 20× objective and a Leica DM6000 epifluorescence microscope (Leica Microsystems, Wetzlar, Germany) or a Leica SP5 confocal microscope (Leica Microsystems, Wetzlar, Germany) equipped with a 63× oil objective using a 3× digital zoom and 0.5-0.8 sequential scanning z-step interval.

Intravitreal Injections

Following a 7-10 day acclimatisation period, 12 week old C57/BL.6 or 16 week old P301S (Harlan labs, Bicester, U.K.) mice were randomised into various study groups. They were then anaesthetized with intraperitoneal injection of ketamine (50 mg/kg) and xylazine (5 g/kg). Topical 1% tetracaine eye drops were administered on Day 1 of the study. Pupillary dilation was achieved using 1% tropicamide eye drops. Using an operating microscope, a partial-thickness scleral pilot hole was made with a 30-gauge needle to facilitate penetration of the underlying sclera, choroid, and retina by a fine metal micropipette with a tip diameter of 30 μm and a tip length of 2.5 mm. The micropipette was then connected to a 10 μL glass syringe (Hamilton Co., Reno, Nev.) prior drawing up 2 μL of vector suspensions into the pipette depending on the group. Care was taken to avoid penetration of the lens or damage to the vortex veins during intravitreal injection. The injection site was aimed approximately 3 mm posterior to the supero-temporal limbus. Injections were given slowly over 1 minute to allow diffusion of vector suspension. The right eye was left untouched and served as an internal contralateral control.

Optic Nerve Crush (ONC)

Three weeks (21 days) after vector administration, the mice were subject to the ONC procedure, left untreated or sham-crushed. Under a binocular operating scope, a small incision was made with spring scissors in the conjunctiva beginning inferior to the globe and around the eye temporally. This exposed the posterior aspect of the globe, allowing visualization of the optic nerve. The exposed optic nerve was grasped approximately 1-3 mm from the globe with cross-action forceps (Dumont #N7 cat. #RS-5027; Roboz) for 10 s, with the only pressure from the self-clamping action to press on the nerve. After 10 s the optic nerve was released, the forceps are removed and the eye rotates back into place. 7 days after ONC, animals were culled. Both eyes from each group were fixed by placing the organ in 4% paraformaldehyde/0.1% PBS (pH 7.4) overnight. Retinal flat-mounts were then prepared following dissection of the posterior eye structure from the cornea and removal of the lens. The retinal flat-mounts were post fixed for 30 minutes in 4% paraformaldehyde/0.1% PBS and washed in 0.5% Triton X-100 in PBS. Retinas were frozen at −80° C. for 10 minutes to permeate the nuclear membrane and improve antibody permeation before blocking in 10% normal donkey serum (NDS), 2% bovine serum albumin (BSA) and 2% Triton X-100 in PBS for 60 minutes at room temperature. RGCs were counterstained with antibodies against Brn3A (1:200 Santa Cruz, #sc-31984) and visualised under fluorescence microscopy using a 20× objective and a Leica DM6000 epifluorescence microscope (Leica Microsystems, Wetzlar, Germany). Higher resolution images were be obtained using a Leica SP5 confocal microscope (Leica Microsystems) equipped with a 40× oil objective using a 1.5× digital zoom and 0.5-0.8 sequential scanning z-step interval. RGC cell counts were measured by ImageJ using the image-based tool for counting nuclei plugin (ITCN) and expressed as density of RGCs/mm².

Constructs and Vectors

The inventors have generated a genetic construct, as shown in FIG. 1, which may be used to treat a subject afflicted with an optic nerve pathology, such as glaucoma, or a cochlear pathology, or for promoting nerve regeneration and/or survival. The construct has been designed to maintain or increase the density of TrkB receptors on the cell surface of RGCs and maintain or increase signaling through the TrkB receptor pathway by concomitant production and local release of mBDNF.

The construct comprises transgenes encoding the TrkB receptor and its agonist, mature brain-derived neurotrophic factor. These transgenes are operably-linked to a single promoter, which is either the human synapsin I (SYN I) promoter or the CAG promoter. Advantageously, the construct of FIG. 1 can be placed in a rAAV2 vector without being hindered by the size of the transgenes that it encodes. This is because the construct is orientated such that the first transgene, TrkB, is linked to the viral 2A peptide sequence followed by the BDNF signal peptide and then the mature protein. This orientation also minimises immunogenicity risks because the short N-terminal amino acid sequence of the viral 2A peptide remains attached to the intracellular portion of the TrkB receptor and the residual proline amino acid from the C-terminal viral 2A sequence remains attached to the N-terminal BDNF signal peptide and is ultimately removed from the mBDNF protein following cleavage. The vector may be placed in a pharmacologically acceptable buffered solution, which may be administered to a subject.

FIGS. 2-5 show various embodiments of expression vectors. FIG. 2 shows the vector known as “Plasmid QTA001PA” containing canonical signal sequence (blue) (i.e. MTILFLTMVISYFGCMKA [SEQ ID NO:20]) plus proBDNF (red) and mBDNF (black). FIG. 3 shows the vector known as “Plasmid QTA002P”. It does not encode proBDNF but produces only mBDNF, and encodes the same signal sequence (blue) as QTA001PA. FIG. 4 shows the vector known as “Plasmid QTA003P” which also does not encode proBDNF but produces only mBDNF. Instead of the canonical signal sequence for mBDNF, it comprises an IL-2 signal sequence (blue). Finally, FIG. 5 shows the vector known as “Plasmid QTA004P”. It does not encode proBDNF but instead produces only mBDNF. It also encodes a novel signal sequence (blue), [SEQ ID NO: 32].

The inventors have produced and investigated the construct and vector relating to the glaucoma gene therapy concept starting with the mature BDNF (mBDNF) element. They have clearly demonstrated production and release of mBDNF from HEK293 cells following lipofectamine transduction with a plasmid which contains the BDNF sequence without the proBDNF coding region (QTA002P, see FIG. 3) (see FIG. 7). The mBDNF released from the cells is the predicted 14 kDa monomer (measured using Western blotting and a commercially available antibody for BDNF) and there is no evidence for protein aggregates, as has been reported by several groups attempting to generate commercial amounts of mBDNF using yeast and other cell-based manufacturing approaches¹. The mBDNF is therefore released in a form which can allow the protein molecules to form non-covalent dimers in order to activate TrkB receptors.

Using an ELISA for BDNF (which does not differentiate between mBDNF and the larger extended proBDNF protein), the inventors have also demonstrated that it is possible to substitute the DNA sequence coding for the endogenous canonical 18-amino acid signal peptide sequence (MTILFLTMVISYFGCMKA) with a novel peptide sequence (QTA004P—see FIG. 5) and release equivalent levels of BDNF into the HEK293 incubation medium following lipofectamine transduction of the cells with plasmids containing the BDNF gene (see FIG. 7).

Substitution of the endogenous signal peptide with the sequence coding for the interleukin-2 signal peptide (QTA003P—see FIG. 4) was less effective in releasing BDNF from the medium. Levels of BDNF released into the medium are currently around 1-2 nM and concentrations of this agonist are sufficient to maximally activate the specific TrkB receptors (IC50 of around 0.9 nM). Levels of BDNF release are approximately 35-fold higher (876±87 ng/mL BDNF) with the plasmid QTA001PA (see FIG. 2) which contains the combined proBDNF and mBDNF sequences and which also includes the 18-amino acid canonical signal peptide as compared to the plasmids QTA002P (see FIG. 3) and QTA004P (see FIG. 5).

Measurements of BDNF remaining in the cell by quantitative Western blotting 24 hours after lipofectamine plasmid transduction revealed lower BDNF remaining concentrations with QTA001PA than those with QTA002P and QTA004P (see FIG. 8).

Moreover, around half of the BDNF immunoreactivity in the cell lysates transduced by QTA001PA was in the form of the proBDNF (molecular weight band at 32 kDa) whereas the proBDNF band was absent in the lysates of cells transduced with QTA002P, QTA003P and QTA004P (see FIG. 9), probably because these plasmids do not contain a proBDNF extended coding sequence.

Using an ELISA specific for the proBDNF, the inventors were able to demonstrate that around 70 ng/mL (2.2 nM or 3.5%) of released BDNF-immunoreactivity from cells transduced by QTA001PA is in the form of proBDNF whilst the majority (96.5% or 876 ng/mL/63 nM) is released as mBDNF (see FIG. 10). There was no proBDNF-immunoreactivity detected from cells transduced by QTA002P, QTA003P or QTA004P which do not contain the coding sequence for the extended proBDNF.

Accordingly, it is clear that all of the plasmids are capable of producing the 14 kDa mBDNF protein, but that the amounts of mBDNF released from the HEK293 cells are largely dependent on efficiency in protein storage and packaging into secretory vesicles. The extended form of the protein, containing the combined proBDNF and mBDNF sequences, as produced with plasmid QTA001PA (FIG. 2) is therefore packaged into secretory vesicles and released into the incubation medium much more efficiently than with the smaller mBDNF sequences which appear to accumulate within the cell.

Referring to FIG. 11, it shows that substitution of the coding for the endogenous canonical signal peptide sequence, as represented in plasmid QTA002P, with novel sequences included in plasmids QTA009P to QTA013P increases the concentration of BDNF in HEK293 cells 24 hours after transduction with plasmids. FIG. 12 demonstrates that substitution of the endogenous canonical signal peptide coding sequence included in plasmid QTA002P with novel sequences (plasmids QTA009P to QTA013P) increases release of BDNF (as measured by ELISA) from HEK293 cells, as measured 24 hours after transduction with plasmids.

As shown in FIG. 13, the addition of the viral-2A peptide sequence results in efficient processing of the coding sequence for the large precursor protein into two transgenes, eGFP and BDNF. The Western blots show HEK293 cells 24 hours after they were transduced with plasmids: (i) QTA015P (expressing BDNF and eGFP separated by an IRES spacer), (ii) QTA021P (expressing BDNF followed by eGFP separated by a functional viral-2A peptide sequence), (iii) QTA022P (expressing BDNF followed by eGFP separated by a non-functional viral-2A peptide sequence) and (iv) QTA023P (expressing eGFP followed by coding for BDNF separated by a functional viral-2A peptide sequence).

The coding sequence of QTA021P (plasmid containing codon optimised sequence for mBDNF-viral-2A peptide-eGFP) is referred to here as SEQ ID No: 104, as follows:

[SEQ ID No: 104]
ATGACTATCCTGTTTCTGACAATGGTTATTAGCTATTTCGGTTGCATGAA
GGCTCACAGTGATCCCGCACGCCGCGGAGAACTTAGCGTGTGCGACAGCA
TCAGCGAGTGGGTCACCGCCGCCGATAAGAAGACCGCTGTGGATATGTCC
GGCGGGACCGTCACTGTACTCGAAAAAGTTCCAGTGAGCAAAGGCCAACT
GAAACAATATTTCTATGAAACTAAGTGCAACCCCATGGGGTACACCAAGG
AGGGCTGCCGGGGAATCGACAAGAGACACTGGAATTCCCAGTGCCGGACC
ACTCAGAGCTACGTCCGCGCCTTGACGATGGATTCAAAGAAGCGCATCGG
ATGGCGGTTCATAAGAATCGACACCAGTTGTGTGTGCACGCTGACGATAA
AACGGGGGCGGGCCCCCGTGAAGCAGACCCTGAACTTTGATTTGCTCAAG
TTGGCGGGGGATGTGGAAAGCAATCCCGGGCCAATGGTGAGCAAGGGCGA
GGAGCTGTTCACCGGCGTTGTGCCAATACTGGTTGAGTTGGATGGCGATG
TCAACGGACACAAATTTAGCGTAAGCGGGGAGGGAGAGGGCGACGCCACA
TATGGCAAGCTGACCCTGAAGTTCATTTGCACGACCGGCAAATTGCCCGT
CCCTTGGCCCACACTTGTGACGACCCTGACTTATGGCGTACAGTGCTTCA
GCAGGTACCCTGATCATATGAAGCAACACGACTTCTTTAAGAGTGCCATG
CCAGAGGGATACGTCCAGGAAAGAACCATATTCTTCAAAGATGATGGAAA
TTACAAAACCCGGGCAGAGGTCAAGTTTGAAGGCGACACCCTGGTGAACA
GGATCGAACTCAAAGGCATCGATTTCAAAGAGGACGGAAACATCCTCGGA
CACAAACTGGAATACAATTACAACAGCCACAACGTCTACATCATGGCAGA
TAAACAAAAGAACGGTATTAAAGTGAACTTCAAGATCCGGCACAACATCG
AAGACGGCTCCGTCCAGCTTGCCGACCACTACCAGCAAAATACCCCGATC
GGCGACGGCCCCGTTCTCCTCCCCGATAATCACTACCTGAGTACACAGTC
AGCCTTGAGCAAAGACCCTAATGAAAAGCGGGACCACATGGTTTTGCTGG
AGTTCGTTACCGCAGCGGGTATTACGCTGGGTATGGACGAGCTTTACAAG
TAA

The coding sequence of QTA022P (plasmid containing codon optimised sequence for mBDNF-non-functional viral-2A peptide-eGFP) is referred to here as SEQ ID No: 105, as follows:

[SEQ ID No: 105]
ATGACTATCCTGTTTCTGACAATGGTTATTAGCTATTTCGGTTGCATGAA
GGCTCACAGTGATCCCGCACGCCGCGGAGAACTTAGCGTGTGCGACAGCA
TCAGCGAGTGGGTCACCGCCGCCGATAAGAAGACCGCTGTGGATATGTCC
GGCGGGACCGTCACTGTACTCGAAAAAGTTCCAGTGAGCAAAGGCCAACT
GAAACAATATTTCTATGAAACTAAGTGCAACCCCATGGGGTACACCAAGG
AGGGCTGCCGGGGAATCGACAAGAGACACTGGAATTCCCAGTGCCGGACC
ACTCAGAGCTACGTCCGCGCCTTGACGATGGATTCAAAGAAGCGCATCGG
ATGGCGGTTCATAAGAATCGACACCAGTTGTGTGTGCACGCTGACGATAA
AACGGGGGCGGGCCCCTGTCAAACAAACCCTCAATTTTGACTTGCTGAAG
CTTGCTGGGGATGTCGAGTCCGCTGCCGCGGCTATGGTGAGCAAGGGCGA
GGAGCTGTTCACCGGCGTTGTGCCAATACTGGTTGAGTTGGATGGCGATG
TCAACGGACACAAATTTAGCGTAAGCGGGGAGGGAGAGGGCGACGCCACA
TATGGCAAGCTGACCCTGAAGTTCATTTGCACGACCGGCAAATTGCCCGT
CCCTTGGCCCACACTTGTGACGACCCTGACTTATGGCGTACAGTGCTTCA
GCAGGTACCCTGATCATATGAAGCAACACGACTTCTTTAAGAGTGCCATG
CCAGAGGGATACGTCCAGGAAAGAACCATATTCTTCAAAGATGATGGAAA
TTACAAAACCCGGGCAGAGGTCAAGTTTGAAGGCGACACCCTGGTGAACA
GGATCGAACTCAAAGGCATCGATTTCAAAGAGGACGGAAACATCCTCGGA
CACAAACTGGAATACAATTACAACAGCCACAACGTCTACATCATGGCAGA
TAAACAAAAGAACGGTATTAAAGTGAACTTCAAGATCCGGCACAACATCG
AAGACGGCTCCGTCCAGCTTGCCGACCACTACCAGCAAAATACCCCGATC
GGCGACGGCCCCGTTCTCCTCCCCGATAATCACTACCTGAGTACACAGTC
AGCCTTGAGCAAAGACCCTAATGAAAAGCGGGACCACATGGTTTTGCTGG
AGTTCGTTACCGCAGCGGGTATTACGCTGGGTATGGACGAGCTTTACAAG
TAA

The coding sequence of QTA023P (plasmid containing codon optimised sequence for eGFP-viral-2A peptide-mBDNF) is referred to here as SEQ ID No: 106, as follows:

[SEQ ID No: 106]
ATGGTGAGCAAGGGCGAGGAGCTGTTCACCGGGGTGGTGCCCATCCTGGT
CGAGCTGGACGGCGACGTAAACGGCCACAAGTTCAGCGTGTCCGGCGAGG
GCGAGGGCGATGCCACCTACGGCAAGCTGACCCTGAAGTTCATCTGCACC
ACCGGCAAGCTGCCCGTGCCCTGGCCCACCCTCGTGACCACCCTGACCTA
CGGCGTGCAGTGCTTCAGCCGCTACCCCGACCACATGAAGCAGCACGACT
TCTTCAAGTCCGCCATGCCCGAAGGCTACGTCCAGGAGCGCACCATCTTC
TTCAAGGACGACGGCAACTACAAGACCCGCGCCGAGGTGAAGTTCGAGGG
CGACACCCTGGTGAACCGCATCGAGCTGAAGGGCATCGACTTCAAGGAGG
ACGGCAACATCCTGGGGCACAAGCTGGAGTACAACTACAACAGCCACAAC
GTCTATATCATGGCCGACAAGCAGAAGAACGGCATCAAGGTGAACTTCAA
GATCCGCCACAACATCGAGGACGGCAGCGTGCAGCTCGCCGACCACTACC
AGCAGAACACCCCCATCGGCGACGGCCCCGTGCTGCTGCCCGACAACCAC
TACCTGAGCACCCAGTCCGCCCTGAGCAAGGACCCCAACGAGAAGCGCGA
TCACATGGTCCTGCTGGAGTTCGTGACCGCCGCCGGGATCACTCTCGGCA
TGGACGAGCTGTACAAGGCTCCCGTTAAACAAACTCTGAACTTCGACCTG
CTGAAGCTGGCTGGAGACGTGGAGTCCAACCCTGGACCTATGACCATCCT
TTTCCTTACTATGGTTATTTCATACTTCGGTTGCATGAAGGCGCACTCCG
ACCCTGCCCGCCGTGGGGAGCTGAGCGTGTGTGACAGTATTAGCGAGTGG
GTCACAGCGGCAGATAAAAAGACTGCAGTGGACATGTCTGGCGGGACGGT
CACAGTCCTAGAGAAAGTCCCGGTATCCAAAGGCCAACTGAAGCAGTATT
TCTACGAGACCAAGTGTAATCCCATGGGTTACACCAAGGAAGGCTGCAGG
GGCATAGACAAAAGGCACTGGAACTCGCAATGCCGAACTACCCAATCGTA
TGTTCGGGCCCTTACTATGGATAGCAAAAAGAGAATTGGCTGGCGATTCA
TAAGGATAGACACTTCCTGTGTATGTACACTGACCATTAAAAGGGGAAGA
TAG

Referring to FIG. 14A, there is shown a Western blot of HEK293 cell homogenates 48 hours after transfection with the QTA020V vector. It shows efficient processing of the large precursor coding region which includes the TrkB receptor and BDNF separated by the viral-2A peptide sequence. The two TrkB and mBDNF-immunoreactive transgenes are within in the predicted correct molecular weight sizes. A lack of staining of large precursor protein above the TrkB receptor band should be noted, indicating almost complete or complete processing of the precursor protein in five repeats. FIGS. 14B and 14C show that the transgene proteins produced after vial-2A peptide cleavage have been transported to the correct intracellular compartments in HEK293 cells after processing (TrkB receptors to the cell surface and BDNF to storage vesicles prior to release).

FIG. 15 shows that addition of the viral-2A peptide sequence separating the two coding regions for the TrkB receptor and BDNF results in efficient processing into the two transgenes in mouse retina following intravitreal injection of the rAAV2 vector, QTA020V.

FIG. 16 shows the expression of transgenes in mouse retinal ganglion cell layer as shown by immunocytochemistry following injection of QTA020V, a rAAV2 vector containing the coding for the TrkB receptor and BDNF, separated by the viral-2A peptide sequence. Target retinal ganglion cell bodies are stained red with anti-Brn3A antibodies and cell nuclei are counter-stained blue with DAPI to distinguish the retinal layers.

Referring to FIG. 17, there is shown pre-treatment of QTA020V (containing coding for TrkB receptor and BDNF, separated by the viral-2A peptide sequence) via intravitreal injection (21 of 9×10¹² vector particles/ml) imparts significant neuroprotective efficacy on retinal ganglion cell survival following optic nerve crush in the mouse versus control animals treated with rAAV2-CAG-eGFP vector. The level of neuroprotection by the QTA020V vector was also greater than that provided by a vector expressing only BDNF. All three groups of animals were subjected to optic nerve crush procedure and the number of retinal ganglion cells measured 7 days after the insult. Retinal ganglion cells were reduced by 71% in controls (black bars) versus animals subject to sham crush (data not shown).

The Neuroprotective Effects of the Constructs

Referring to FIG. 18, there are shown the expression of the BDNF transgenes (see FIG. 18A) and the TrkB transgenes (see FIG. 18B) in undifferentiated human SH-SY5Y neuroblastoma cell homogenates by Western blotting following transfection with rAAV2 viral vectors which express no transgenes (Null virus), BDNF only (QTA027V), TrkB only (QTA025V) and both BDNF and TrkB (QTA020V). It is clear that good levels of expression are achieved.

Referring to FIG. 18C, there is shown the level of activated phosphorylated TrkB receptors in the SH-SY5Y cells in Western blots following transfection with the viral vectors Null, QTA020V, QTA025V or QTA027V. Only QTA020V vector which expresses both BDNF and TrkB was found to significantly increase the activation of TrkB receptors, as compared to untransfected cells. As such, it has been shown that the constructs of the invention effectively express both transgenes and result in activated phosphorylated TrkB receptors in the neuroblastoma SH-SY5Y cells, indicating that neurodegenerative disorders, such as Alzheimer's disease, or stroke, can be treated.

Referring to FIG. 19, there is shown the level of apoptotic cell death of undifferentiated neuroblastoma SH-SY5Y cells in culture following exposure to oxidative stress produced by addition of hydrogen peroxide (H₂O₂ at either 0.1 mM or 1.0 mM) by TUNEL staining. Cells transfected with the rAAV2 vector QTA020V, which expresses both BDNF and TrkB receptors, prior to addition of the hydrogen peroxide, were surprisingly found to be significantly protected against apoptosis versus untreated cells. Again, these data support the notion that the constructs of the invention can be used in the treatment, prevention or amelioration of a neurodegenerative disorder or stroke.

Referring now to FIG. 20, there are shown representative immunocytochemical images of optic nerves obtained from P301S mutant human Tau transgenic mice and stained with antibodies which recognise phosphorylated Tau at positions serine 396/serine 404 (PHF-1) or serine 202/serine 205 (AT8).

P301S transgenic mice develop neuronal loss and brain atrophy by eight months, principally in the hippocampus but spreading to other brain regions, including the neocortex and entorhinal cortex. They develop widespread neurofibrillary tangle-like inclusions in the neocortex, amygdala, hippocampus, brain stem, and spinal cord. Tangle pathology is accompanied by microgliosis and astrocytosis, but not amyloid plaques [56, 57,58].

Mice were treated via intravitreal injection with QTA020V which expresses both TrkB receptors and BDNF in target retinal ganglion cells and their axons. The images in FIG. 20 illustrate that the degree of Tau hyperphosphorylation, using PHF-1 and AT-8, is significantly reduced in the axons that constitute the optic nerve. These in vivo data show that increased expression of TrkB and BDNF, using the constructs of the invention, can significantly reduce Tau phosphorylation in neurones, which is one of the pathophysiological features associated with Alzheimer brains.

### CONCLUSIONS

It will be appreciated that for Alzheimer's disease, there is no single pre-clinical model, which is generally regarded as a surrogate for the disease and where a gene therapy may be tested with a degree of predictability towards a clinical outcome. What are available, however, are animals models in which modifications to their genome have resulted in the introduction of one of the defining genetic/neurochemical or biochemical changes into rodents which have been identified in humans with the disease. These changes include the excessive production of Aβ and formation of plaques [59] generation of a hyper-phosphorylated tau protein within neuronal cell bodies and axons which are thought to mediate axonal transport [60] and the reduction in both BDNF and its cognate receptor, TrkB [11-14, 27]

Based on human post-mortem tissue and the ability of various agents which can successfully remove beta-amyloid from both experimental animals through blockade of the BACE-1 enzyme responsible for its generation (verubecestat; Merck) or through antibody neutralisation (e.g. solenezumab; Eli Lilly and bapineuzumab; Pfizer/J&J), both of these approaches have failed to produce significant clinical benefit in Phase-III clinical studies. Therefore, of the widely described post-mortem changes in human brains diagnosed with Alzheimer's disease, loss in BDNF signalling and the presence of neurofibrillary tangles associated with hyper-phosphorylated tau are the only untested approaches to restoring or slowing pathophysiological changes associated with this neurological condition.

Using significant inventive endeavour, the inventors have addressed the problem of overcoming the loss in BDNF signalling using a novel construct which is simultaneously able to both express and up-regulate both TrkB receptors and BDNF, both of which have been reported to be reduced in this disease (see references cited above).

As BDNF has a short half-life, regular administration of recombinant BDNF, which may require several injections per day into the brain or through constant infusion, is clinically not feasible and would probably be associated with TrkB receptor down-regulation. Moreover, the inventors have also demonstrated in FIG. 18C that in SHSY-5Y cells, an rAAV2 expressing TrkB receptors alone is not sufficient to significantly increase the activity of this receptor, as measured by the levels of active p-Y⁵¹⁵-TrkB staining. The constructs of the invention, which have been specifically designed to accommodate the large coding sequences of both TrkB receptor and BDNF through a number of inventive steps including: (i) loss of pro-BDBF coding, (ii) introduction of a novel signal peptide to overcome the issues associated with intracellular transport and normal protein folding of BDNF due to omission of the important Pro-BDNF sequence, (iii) constructing a single transgene containing a viral-2A peptide sequence which facilitates translational ‘skipping’ between the ribosomal production of TrkB and the BDNF sequences, and (iv) finally abbreviated WPRE and polyA sequences. Therefore, the inventors have provided evidence that the novel construct which expresses two transgenes, BDNF, and its cognate receptor, BDNF, is far superior to simply up-regulating TrkB receptors alone. The inventors have also demonstrated that the novel gene therapy constructs are able to provide optimal activity, as has been previously demonstrated [56], but without the requirement for additional (regular) injections of BDNF.

The inventor's main objective was to develop a gene therapy which is capable of addressing the low levels of BDNF/TrkB signalling which the examples provided clearly demonstrate. What was unexpected was that the novel gene therapy construct is capable of a major reduction in the density of hyper-phosphorylated Tau protein (measured using two antibodies which recognise several phosphorylated serine residues along the Tau protein length), as shown in FIG. 20. Tau is a ubiquitous protein found in brain and other neural tissues, such as the optic nerve. Using the optic nerve as a model system, increased BDNF signalling in the eye was found to reduce the proposed pathological level of this protein isoform. Therefore, the ability to up-regulate the BDNF/TrkB signalling in the P301S transgenic mouse strain and observe such a profound reduction in the density of phosphorylated-Tau was not anticipated.

### REFERENCES

1. Brookmeyer R, Johnson E, Ziegler-Graham K, Arrighi H M. Forecasting the global burden of Alzheimer's disease (2007). Alzheimers. Dement., vol. 3(3) PP:186-191.
2. Braak H, Braak E. (1991). Neuropathological staging of Alzheimer-related changes. Acta Neuropathol., vol. 82(4), PP:239-259.
3. Dekosky S T, Scheff S W. (1990). Synapse loss in frontal cortex biopsies in Alzheimer's disease: correlation with cognitive severity. Ann. Neurol., vol. 27(5), PP:457-464.
4. Chao M V (2003). Neurotrophins and their receptors: A convergence point for many signalling pathways. Nature Rev. Neurosci. vol. 4, PP: 299-309.
5. Dawbarn D. Allen S J, (2003). Neurotrophins and neurodegeneration. Neuropathol. Appl. Neurobiol., vol. 29, PP: 211-230.
6. Phillips H S, Hains J M, Laramee G R, Rosenthal A, Winslow J W (1990). Widespread expression of BDNF but not NT3 by target areas of basal forebrain cholinergic neurons. Science, vol. 250(4978), PP: 290-294.
7. Wetmore C, Ernfors P, Persson H, Olson L, (1990). Localization of brain-derived neurotrophic factor mRNA to neurons in the brain by in situ hybridization. Exp. Neurol., vol. 109(2), PP: 141-152
8. Ghosh A, Carnahan J, Greenberg M E, (1994). Requirement for BDNF in activity-dependent survival of cortical neurons. Science, vol. 263(5153), PP: 1618-1623.
9. Phillips H S, Hains J M, Armanini M, Laramee G R, Johnson S A, Winslow J W, (1991). BDNF mRNA is decreased in the hippocampus of individuals with Alzheimer's disease. Neuron, vol. 7(5), PP: 695-702.
10. Holsinger R M, Schnarr J, Henry P, Castelo V T, Fahnestock M, (2000). Quantitation of BDNF mRNA in human parietal cortex by competitive reverse transcription-polymerase chain reaction: decreased levels in Alzheimer's disease. Brain Res. Mol. Brain Res., vol. 76(2), PP: 347-354.
11. Narisawa-Saito M, Wakabayashi K, Tsuji S, Takahashi H, Nawa H, (1996). Regional specificity of alterations in NGF, BDNF and NT-3 levels in Alzheimer's disease. Neuroreport, vol. 7(18), PP: 2925-2928.
12. Connor B, Young D, Yan Q, Faull R L, Synek B, Dragunow M, (1997). Brain-derived neurotrophic factor is reduced in Alzheimer's disease. Brain Res. Mol. Brain Res., vol. 49(1-2), PP: 71-81.
13. Ferrer I, Marin C, Rey M J, Ribalta T, Goutan E, Blanco R, Tolosa E, Marti E, (1999). BDNF and full-length and truncated TrkB expression in Alzheimer disease. Implications in therapeutic strategies. J. Neuropathol. Exp. Neurol., vol. 58(7), PP: 729-739.
14. Savaskan E, Muller-Spahn F, Olivieri G, Bruttel S, Otten U, Rosenberg C, Hulette C, Hock C, (2000). Alterations in trk A, trk B and trk C receptor immunoreactivities in parietal cortex and cerebellum in Alzheimer's disease. Eur. Neurol., vol. 44(3), PP: 172-180.
15. Peng S, Wuu J, Mufson E J, Fahnestock M, (2005). Precursor form of brain-derived neurotrophic factor and mature brain-derived neurotrophic factor are decreased in the pre-clinical stages of Alzheimer's disease. J. Neurochem., vol. 93(6) PP: 1412-1421.
16. Michalski B, Fahnestock M, (2003). Pro-brain-derived neurotrophic factor is decreased in parietal cortex in Alzheimer's disease. Brain Res. Mol. Brain Res., vol. 111(1-2), PP: 148-154.
17. Qin X Y, Cao C, Cawley N X, Liu T, Yuan J, Loh Y P, Cheng Y, (2016). Decreased peripheral brain-derived neurotrophic factor levels in Alzheimer's disease: A meta-analysis study (N=7277). Mol. Psychiatry, vol. 22, PP: 312-320.
18. Forlenza O V, Diniz B S, Teixeira A L, Radanovic M, Talib L L, Rocha N P, Gattaz W F, (2015). Lower Cerebrospinal Fluid Concentration of Brain-Derived Neurotrophic Factor Predicts Progression from Mild Cognitive Impairment to Alzheimer's Disease. Neuromolecular Med., vol. 17(3), PP: 326-332.
19. Feher A, Juhasz A, Rimanoczy A, Kalman J, Janka Z, (2009). Association between BDNF Val66Met polymorphism and Alzheimer disease, dementia with Lewy bodies, and Pick disease. Alzheimer Dis. Assoc. Disord., vol. 23(3) PP: 224-228.
20. Peng S, Wuu J, Mufson E J, Fahnestock M, (2005). Precursor form of brain-derived neurotrophic factor and mature brain-derived neurotrophic factor are decreased in the pre-clinical stages of Alzheimer's disease. J. Neurochem., vol. 93, PP: 1412-1421.
21. Michalski B, Corrada M M, Kawas C H, Fahnestock M, (2015). Brain-derived neurotrophic factor and TrkB expression in the “oldest-old”, the 90+ study: Correlation with cognitive status and levels of soluble amyloid-β. Neurobiol. Aging, vol. 36, PP: 3130-3139.
22. Elliott E, Atlas R, Lange A, Ginzburg I, (2005). Brain-derived neurotrophic factor induces a rapid dephosphorylation of tau protein through a PI-3 kinase signalling mechanism. Eur. J. Neurosci., vol. 22, PP: 1081-1089.
23. Jiao S-S, Shen L-L, Zhu C, Bu X-L, Liu Y-H, Liu C-H, Yao X-Q, Zhang L-L, Zhou H-D, Walker D G, Tan J, Gortz J, Zhou X-F, Wang Y-J, (2016). Brain-derived neurotrophic factor protects against tau-related neurodegeneration of Alzheimer's disease. Transl. Psychiatry, vol. 6, e907.
24. Rosa E, Mahendram S, Ke Y D, Ittner L M, Ginsberg S D, Fahnestock M, (2016). Tau downregulates BDNF expression in animal and cellular models of Alzheimer's disease. Neurobiol. Aging, vol. 48,PP: 135-142.
25. Lim J Y, Reighard C P, Crowther D C, (2015). The pro-domains of neurotrophins, including BDNF, are linked to Alzheimer's disease through a toxic synergy with Aβ. Hum. Mol. Genet., vol., 24(14) PP:3929-3938.
26. Hashimoto R, Hirata Y, Asada T, Yamashita F, Nemoto K, Mori T, Moriguchi Y, Kunugi H, Arima K, Ohnishi T, (2009). Effect of the brain-derived neurotrophic factor and the apolipoprotein E polymorphisms on disease progression in preclinical Alzheimer's disease. Genes Brain Behav., vol. 8(1) PP: 43-52.
27. Allen S J, Wilcock G K, Dawbarn D, (1999). Profound and selective loss of catalytic TrkB immunoreactivity in Alzheimer's disease. Biochem. Biophys. Res. Commun., vol. 264(3) PP: 648-651.
28. Kemppainen S, Rantamaki T, Jeronimo-Santos A, Lavasseur G, Autio H, Karpova N, Karkkainen E, Staven S,Vicente Miranda H, Outeiro T F, Diógenes M J, Laroche S, Davis S, Sebastião A M, Castrén E, Tanila H, (2012). Impaired TrkB receptor signaling contributes to memory impairment in APP/PS1 mice. Neurobiol. Aging, vol. 33, e1123-e1139.
29. Devi L, Ohno M (2015) TrkB reduction exacerbates Alzheimer's disease-like signalling aberrations and memory deficits without affecting β-amyloidosis in 5XFAD mice. Transl. Psychiatry, vol. 5, e562.
30. Tong L, Balazs R, Thornton P L, Cotman C W, (2004). β-Amyloid peptide at sublethal concentrations downregulates brain-derived neurotrophic factor functions in cultured cortical neurons. J. Neurosci., vol. 24, PP:6799-6809.
31. Liu X H, Geng Z, Yan J, Li T, Chen Q, Zhang Q Y, Chen Z Y, (2015). Blocking GSK33-mediated dynamin1 phosphorylation enhances BDNF-dependent TrkB endocytosis and the protective effects of BDNF in neuronal and mouse models of Alzheimer's disease. Neurobiol. Dis., vol. 74, PP: 377-391.
32. Poon W W, Blurton-Jones M, Tu C H, Feinberg L M, Chabrier M A, Harris J W, Jeon N L, Cotman C W, (2011). β-amyloid impairs axonal BDNF retrograde trafficking. Neurobiol. Aging, vol. 32, PP: 821-833.
33. Gong B, Cao Z, Zheng P, Vitolo O V, Liu S, Staniszewski A, Moolman D, Zhang H, Shelanski M, Arancio O, (2006). Ubiquitin hydrolase Uch-L1 rescues β-amyloid-induced decreases in synaptic function and contextual memory. Cell, vol. 126, PP: 775-788.
34. Gan K J, Silverman M A, (2015). Dendritic and axonal mechanisms of Ca2+ elevation impair BDNF transport in aβ oligomer-treated hippocampal neurons. Mol. Biol. Cell, vol. 26, PP: 1058-1071.
35. Zhang L, Fang Y, Lian Y, Chen Y, Wu T, Zheng Y, Zong H, Sun L, Zhang R, Wang Z, Xu Y, (2015). Brain-derived neurotrophic factor ameliorates learning deficits in a rat model of Alzheimer's disease induced by aβ1-42., PLoS One, vol. 10(4), eo0122415.
36. Wu Y, Luo X, Liu X, Liu D, Wang X, Guo Z, Zhu L, Tian Q, Yang X, Wang J Z, (2015). Intraperitoneal Administration of a Novel TAT-BDNF Peptide Ameliorates Cognitive Impairments via Modulating Multiple Pathways in Two Alzheimer's Rodent Models. Sci. Rep., vol. 5, PP: 15032.
37. Nagahara A H, Merrill D A, Coppola G, Tsukada S, Schroeder B E, Shaked G M, Wang L, Blesch A, Kim A, Conner J M, Rockenstein E, Chao M V, Koo E H, Geschwind D, Masliah E, Chiba A A, Tuszynski M H, (2009). Neuroprotective effects of brain-derived neurotrophic factor in rodent and primate models of Alzheimer's disease. Nat. Med., vol. 15(3), PP: 331-337.
38. Mey J, Thanos S, (1993). Intravitreal injections of neurotrophic factors support the survival of axotomized retinal ganglion cells in adult rats in vivo. Brain Res., vol. 602, PP: 304-317.
39. Mansour-Rabaey S, Clarke D B, Wang Y-C, Bray G M, Aguayo A J, (1994). Effects of ocular injury and administration of brain-derived neurotrophic factor on survival and regrowth of axotomized retinal ganglion cells. Proc. Nat. Acad. Sci. USA., vol. 91, PP: 1632-1636.
40. Peinado-Ramon P, Salvador M, Vi\ddot{υ}egas-Perez M P, Vidal-Sanz M, (1996). Effects of axotomy and intraocular administration of NT-4, NT-3 and brain-derived neurotrophic factor on the survival of adult rat retinal ganglion cells. A quantitative in vivo study. Invest Ophthalmol. Vis. Sci., vol. 37, PP: 489-500.
41. Di Polo A, Aigner L J, Dunn R J, Bray C M, Aguayo A I, (1998). Prolonged delivery of brain-derived neurotrophic factor by adenovirus-infected Müller cells temporarily rescues injured retinal ganglion cells. Proc. Natl. Acad. Sci. USA., vol. 95, PP: 3978-3983.
42. Klocker N, Kermer P, Weishaupt J H, Labes M, Ankerhold R, Bihr M, (2000). Brain-derived neurotrophic factor-mediated neuroprotection of adult rat retinal ganglion cells in vivo does not exclusively depend on phosphatidyl-inositol-3′-kinase/protein kinase B signaling. J. Neurosci., vol. 20, PP: 6962-6967.
43. Ko M L, Hu D N, Ritch R, Sharma S C, Chen C F, (2001). Patterns of retinal ganglion cell survival after brain-derived neurotrophic factor administration in hypertensive eyes of rats. Neurosci. Lett., vol. 305, PP: 139-142.
44. Chen H, Weber A J, (2001). BDNF enhances retinal ganglion cell survival in cats with optic nerve damage. Invest Ophthamol. Vis. Sci., vol. 42, PP: 966-974.
45. Pórez M T R, Caminos E, (1995). Expression of brain-derived neurotrophic factor and its functional receptor in neonatal and adult rat retina. Neurosci. Lett., vol. 183, PP: 96-99.
46. Vecino E, Ugarte M, Nash M S, Osborne N N. (1999). NMDA induces BDNF expression in the albino rat retina in vivo. Neuroreport., vol. 10, PP: 1103-1106.
47. Mowla S J, Farhadi H F, Pareek S, Atwal J K, Morris S J, Seidah N G, Murphy R A, (2001). Biosynthesis and post-translational processing of the precursor to brain-derived neurotrophic factor. J. Biol. Chem., vol. 276, PP: 12660-12666.
48. Gupta V K, You Y, Gupta V B, Klistorner A, Graham S L, (2013). TrkB receptor signalling: Implications in neurodegenerative, psychiatric and proliferative disorders. Int. J. Mol. Sci., vol. 14, PP: 10122-10142
49. Teng H K, Teng K K, Lee R, Wright S, Tevar S, Almeida R D, Kermani P, Torkin R, Chen Z Y, Lee F S, Kraemer R T, Nykjaer A, Hempstead B L, (2005). ProBDNF induces neuronal apoptosis via activation of a receptor complex of p75NTR and sortilin. J. Neurosci., vol. 25, PP: 5455-5463.
50. Wei Y, Zhang F, Zao J, Jiang X, Lu Q, Gao E, Wand N, (2012). Enhanced protein expression of proBDNF and proNGF in elevated intraocular pressure-induced rat retinal ischemia. Chin. Med. J., vol. 125, PP: 3875-3879.
51. Woo N H, Teng H K, Siao C-J, Chiaruttini C, Pang P T, Milner T A, Hempstead B L,Lu B, (2005). Activation of p75^{NTR} by proBDNF facilitates hippocampal long-term depression. Nature Neurosci., vol. 8, PP: 1069-1077.
52. Lebrun-Julien F, Bertrand M J, De Backer O, Stellwagen D, Morales C R, Di Polo A, Barker P A, (2010). ProNGF induces TNFalpha-dependent death of retinal ganglion cells through a p75NTR non-cell-autonomous signaling pathway. Proc. Natl. Acad. Sci. USA., vol. 107, PP: 3817-3822.
53. Quigley H A, McKinnon S J, Zack D J, Pease M E, Kerrigan-Baumrind L A, Kerrigan D F, Mitchell R S, (2000). Retrograde axonal transport of BDNF in retinal ganglion cells is blocked by acute IOP elevation in rats. Invest. Ophthalmol. Vis. Sci., vol. 41, PP: 3460-3466.
54. Pease M E McKinnon S J, Quigley H A, Kerrigan-Baumrind L A, Zack D J, (2000). Obstructed axonal transport of BDNF and its receptor TRKB in experimental glaucoma. Invest. Ophthalmol. Vis. Sci., vol. 41, PP: 764-774.
55. Wei Y, Wang N, Lu Q, Zhang N, Zheng D, Li J, (2007). Enhanced protein expressions of sortilin and p75NTR in retina of rat following elevated intraocular pressure-induced retinal ischemia. Neurosci. Lett., vol. 429, PP: 169-174.
56. Cheng L, Sapieha P, Kittlerova P, Hauswith W W, Di Polo A, (2002). TrkB gene transfer protects retinal ganglion cells from axotomy-induced death in vivo. J. Neurosci., vol. 22(10), PP: 3977-3986.
57. YoshiyamaY, Higuchi M, Zhang B, Huang S M, Iwata N, Saido T C, Maeda J, Suhara T, Trojanowski T C, Lee V M, (2007). Synapse loss and microglial activation precede tangles in a P301S Tauopathy mouse model. Neuron, vol. 53, PP: 337-351.
58. Takeuchi H, Iba M, Inoue H, Higuchi M, Takao K, Tsukita K, Karatsu Y, Iwamoto Y, Miyakama T, Suhara T, Trojanowski J Q, Lee V M, Takahashi R, (2011). P301S mutant human Tau transgenic mice manifest early symptoms of human tauopathies with dementia and altered sensorimotor gating. PLoS One, vol. 6(6), e21050.
59. Hardy J, Allsop D, (1991). Amyloid deposition as the central event in the aetiology of Alzheimer's disease, Trends Pharmacol. Sci., vol. 12(10), PP:383-388.
60. Mudher A, Lovestone S, (2002). Alzheimer's disease—do tauists and baptists finally shake hands?. Trends Neurosci., vol. 25(1), PP: 22-26.

1. A method of treating, preventing or ameliorating a neurodegenerative disorder in a subject, the method comprising administering, to a subject in need of such treatment, a therapeutically effective amount of a recombinant vector comprising a genetic construct comprising a promoter operably linked to a first coding sequence, which encodes the tyrosine kinase receptor B (TrkB), and a second coding sequence, which encodes an agonist of the TrkB receptor, wherein the agonist is mature BDNF or mature NT-4, wherein the second coding sequence comprises a nucleotide sequence encoding a signal peptide which boosts secretion of the agonist of the TrkB receptor, and wherein the genetic construct comprises a spacer sequence disposed between the first and second coding sequences, which spacer sequence encodes a peptide spacer that is configured to be digested to thereby produce the TrkB receptor and agonist as separate molecules.

2-3. (canceled)

4. The method according to claim 1, wherein the promoter is the human synapsin I (SYN I) or the CAG promoter.

5. (canceled)

6. The method according to claim 1, wherein the spacer sequence comprises and encodes a viral peptide spacer sequence, optionally a viral 2A peptide spacer sequence.

7. The method according to claim 1, wherein the peptide spacer sequence comprises an amino acid sequence as set out in SEQ ID NO. 4,
or a fragment or variant with at least 65% sequence identity to SEQ ID No: 4; or (ii) an amino acid sequence as set out in SEQ ID NO. 6, or a fragment or variant with at least 65% sequence identity to SEQ ID No: 6; or (iii) an amino acid sequence as set out in SEQ ID NO. 8, or a fragment or variant with at least 65% sequence identity to SEQ ID No: 8.

8. The method according to claim 1, wherein the spacer sequence comprises a nucleotide sequence as set out in SEQ ID NO.5, or a fragment or variant thereof with at least 65% sequence identity to SEQ ID No: 5; or (ii) a nucleotide sequence as set out in SEQ ID NO. 7, or a fragment or variant with at least 65% sequence identity to SEQ ID No: 7.

9-11. (canceled)

12. The method according to claim 1, wherein the first coding sequence comprises a nucleotide sequence encoding the human canonical isoform of TrkB, wherein the canonical isoform of TrkB comprises an amino acid sequence as set out in SEQ ID NO. 9, or a fragment or variant at least 65% sequence identity to SEQ ID No: 9.

13. The method according to claim 1, wherein the first coding sequence comprises a nucleotide sequence as set out in SEQ ID NO. 10, or a fragment or variant with at least 65% sequence identity to SEQ ID No: 10.

14. The method according to claim 1, wherein the first coding sequence comprises a nucleotide sequence which encodes isoform 4 of TrkB, and wherein isoform 4 of TrkB comprises an amino acid sequence as set out in SEQ ID NO. 11, or a fragment or variant with at least 65% sequence identity to SEQ ID No: 11.

15. (canceled)

16. The method according to claim 1, wherein the first coding sequence comprises a nucleotide sequence as set out in SEQ ID NO. 12, or a fragment or variant with at least 65% sequence identity to SEQ ID No: 12.

17. The method according to claim 1, wherein the first coding sequence encoding an amino acid sequence as set out in SEQ ID No: 9, wherein one or more tyrosine residue at position 516, 701, 705, 706 and/or 816 of SEQ ID No: 9 is modified to a different amino acid residue, optionally wherein at least two, three or four tyrosine residues at position 516, 701, 705, 706 and/or 816 of SEQ ID No: 9 are modified to a different amino acid residue or wherein all five tyrosine residues at position 516, 701, 705, 706 and/or 816 of SEQ ID No: 9 are modified to a different amino acid residue.

18-19. (canceled)

20. The method according to claim 17, wherein the or each tyrosine residue is modified to a glutamic acid.

21. The method according to any claim 17, wherein the modified form of the TrkB receptor comprises an amino acid sequence as set out in SEQ ID NO. 13, or a fragment or variant with at least 65% sequence identity to SEQ ID No:
13.

22. The method according to claim 21, wherein the first coding sequence comprises a nucleotide sequence as set out in SEQ ID NO. 14, or a fragment or variant with at least 65% sequence identity to SEQ ID No: 14.

23. The method according to claim 1, wherein the second coding sequence encodes neurotrophin-4 (NT-4), which comprises an amino acid sequence as set out in SEQ ID NO: 49 or 55, or a fragment or variant with at least 65% sequence identity to SEQ ID No: 49 or 55, and/or the second coding sequence comprises a nucleotide sequence as set out in SEQ ID No: 50 or 56, or a fragment or variant with at least 65% sequence identity to SEQ ID No: 50 or 56.

24. The method according to claim 1, wherein the second coding sequence comprises a nucleotide sequence which encodes mature BDNF comprising an amino acid sequence as set out in SEQ ID NO. 18, or a fragment or variant with at least 65% sequence identity to SEQ ID No: 18.

25. (canceled)

26. The method according to claim 1, wherein the second coding sequence comprises a nucleotide sequence as set out in SEQ ID NO. 19, or a fragment or variant with at least 65% sequence identity to SEQ ID No: 19.

27. The method according to claim 1, wherein the second coding sequence comprises a nucleotide sequence encoding a signal peptide for the agonist of the TrkB receptor, optionally a signal peptide for BDNF.

28. The method according to claim 27, wherein the nucleotide sequence encodes the canonical signal peptide for BDNF, wherein the second coding sequence comprises a nucleotide sequence which encodes a signal peptide comprising an amino acid sequence as set out in SEQ ID NO. 20, or a fragment or variant with at least 65% sequence identity to SEQ ID No: 20.

29. The method according to claim 27, wherein the second coding sequence comprises a nucleotide sequence as set out in SEQ ID NO. 21, or a fragment or variant with at least 65% sequence identity to SEQ ID No: 21.

30. The method according to claim 1, wherein the second coding sequence comprises a nucleotide sequence encoding a signal sequence peptide as set out in any one of SEQ ID NO. 23, 25, 27 or 29, or wherein the signal peptide comprises an amino acid sequence as set out in any one of SEQ ID NO. 22, 24, 26 or 28, or wherein the second coding sequence comprises a nucleotide sequence encoding a signal sequence peptide as set out in any one of SEQ ID NO. 31, 33, 35, 37, 39, 41, 43, 45, 61, 63, 65, 67, 69, 71, 73, 75, 77, 79, 81, 83, 85, 87, 89, 91, 93, 95, 97, 99, 101 or 103; or wherein the signal peptide comprises an amino acid sequence as set out in any one of SEQ ID NO. 30, 32, 34, 36, 38, 40, 42, 44, 60, 62, 64, 66, 68, 70, 72, 74, 76, 78, 80, 82, 84, 86, 88, 90, 92, 94, 96, 98, 100 or 102.

31. (canceled)

32. The method according to claim 1, wherein the construct comprises a nucleotide sequence as set out in SEQ ID No: 107 or 108, or a fragment or variant with at least 65% sequence identity to SEQ ID No: 107 or 108.

33. The method according to claim 1, wherein the vector is a recombinant AAV (rAAV) vector, optionally wherein the rAAV is AAV-1, AAV-2, AAV-3A, AAV-3B, AAV-4, AAV-5, AAV-6, AAV-7, AAV-8, AAV-9, AAV-10 or AAV-11.

34. (canceled)

35. The method according to claim 33, wherein the rAAV is rAAV serotype-2.

36. The method according to claim 1, wherein the neurodegenerative disorder is selected from a group consisting of: Alexander's disease, Alper's disease, Alzheimer's Disease, amyotrophic lateral sclerosis (ALS), ataxia telangiectasia, neuronal ceroid lipofuscinoses, Batten disease, bovine spongiform encephalopathy (BSE), Canavan disease, cerebral palsy, Cockayne syndrome, corticobasal degeneration, Creutzfeldt-Jakob disease, frontotemporal lobar degeneration, Gaucher's disease, Huntington's disease, HIV-associated dementia, Kennedy's disease, Krabbe's disease, Lewy body dementia, lysosomal storage disorders, neuroborreliosis, Machado-Joseph disease, motor neurone disease, multiple system atrophy, multiple sclerosis, multiple sulfatase deficiency, mucolipidoses, narcolepsy, Niemann-Pick type C, Niemann Pick disease, Parkinson's Disease, Pelizaeus-Merzbacher Disease, Pick's disease, Pompe disease, primary lateral sclerosis, prion diseases, progressive supranuclear palsy, Refsum's disease, Sandhoff disease, Schilder's disease, subacute combined degeneration of spinal cord secondary to pernicious anaemia, Spielmeyer-Vogt-Sjogren-Batten disease, spinocerebellar ataxia, spinal muscular atrophy, Steele-Richardson-Olszewski disease, Tabes dorsalis, and Tay-Sachs disease.

37. The method according to claim 1, wherein the neurodegenerative disorder is Alzheimer's disease, optionally wherein Tau phosphorylation in neurones is reduced.

38. (canceled)
